SEQUENCE LISTING (198 sequences)

SEQ ID NO: 1 - DNA, 135 bp, Homo sapiens
  misc_feature: CD8alpha hinge - DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 2 - protein, 45 aa, Homo sapiens
  MISC_FEATURE: CD8a hinge - protein
TTTPAPRPPTPAPTIASQPLSLRPEACRPAAGGAVHTRGLDFACD

SEQ ID NO: 3 - DNA, 63 bp, Homo sapiens
  misc_feature: CD8 TM - DNA
atctacatctgggcgcccttggccgggacttgtggggtccttctcctgtcactggttatcacc

SEQ ID NO: 4 - protein, 21 aa, Homo sapiens
  MISC_FEATURE: CD8 TM - protein
IYIWAPLAGTCGVLLLSLVIT

SEQ ID NO: 5 - DNA, 111 bp, Homo sapiens
  misc_feature: OX40 - DNA
cggagggaccagaggctgccccccgatgcccacaagccccctgggggaggcagtttccggacccccatccaagaggagcaggccgacgcccactccaccctggccaagatc

SEQ ID NO: 6 - protein, 37 aa, Homo sapiens
  MISC_FEATURE: OX40 - protein
RRDQRLPPDAHKPPGGGSFRTPIQEEQADAHSTLAKI

SEQ ID NO: 7 - DNA, 336 bp, Homo sapiens
  misc_feature: CD3zeta - DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - protein, 112 aa, Homo sapiens
  MISC_FEATURE: CD3zeta - protein
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, Homo sapiens
  misc_feature: T2A - DNA
ggctctggcgagggaaggggttccctgcttacttgcggcgacgtcgaagagaatcccggtccg

SEQ ID NO: 10 - protein, 21 aa, Homo sapiens
  MISC_FEATURE: T2A - protein
GSGEGRGSLLTCGDVEENPGP

SEQ ID NO: 11 - DNA, 342 bp, Homo sapiens
  misc_feature: IL15 - DNA
aactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtcc

SEQ ID NO: 12 - protein, 114 aa, Homo sapiens
  MISC_FEATURE: IL15 - protein
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - DNA, 207 bp, Homo sapiens
  misc_feature: CD8hinge/TM - DNA
actaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 14 - protein, 66 aa, Homo sapiens
  MISC_FEATURE: CD8hinge/TM - protein
TTTPAPRPPTPAPTIASQPLSLRPEACRPAAGGAVHTRGLDFACDIYIWAPLAGTCGVLLLSLVIT

SEQ ID NO: 15 - DNA, 30 bp, Homo sapiens
  misc_feature: Linker 1 (used after FLAG tag)  - DNA
ggcggtggtggctctggtggtggcggcagc

SEQ ID NO: 16 - protein, 10 aa, Homo sapiens
  MISC_FEATURE: Linker 1 (used after FLAG tag)  - protein
GGGGSGGGGS

SEQ ID NO: 17 - DNA, 30 bp, Homo sapiens
  misc_feature: Linker 2 - after neg. control binding domain - DNA
ggccaggccggctccggaggaggaggatcc

SEQ ID NO: 18 - protein, 10 aa, Homo sapiens
  MISC_FEATURE: Linker 2 - after neg. control binding domain - Protein
GQAGSGGGGS

SEQ ID NO: 19 - DNA, 12 bp, Homo sapiens
  misc_feature: Linker post scFv - DNA
ggccaggccggc

SEQ ID NO: 20 - protein, 4 aa, Homo sapiens
  MISC_FEATURE: Linker post scFv - protein
GQAG

SEQ ID NO: 21 - DNA, 33 bp, Homo sapiens
  misc_feature: Linker - DNA
  misc_feature: Misc Linker - DNA
ggcggcggcggtagcggtggtggcggctccgga

SEQ ID NO: 22 - protein, 11 aa, Homo sapiens
  MISC_FEATURE: Misc Linker - protein
GGGGSGGGGSG

SEQ ID NO: 23 - DNA, 18 bp, Homo sapiens
  misc_feature: Linker - DNA
ggccaggccggctccgga

SEQ ID NO: 24 - protein, 6 aa, Homo sapiens
  MISC_FEATURE: Linker - protein
GQAGSG

SEQ ID NO: 25 - DNA, 405 bp, Homo sapiens
  misc_feature: NKG2D Extracellular Fragment - DNA
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 26 - protein, 135 aa, Homo sapiens
  MISC_FEATURE: NKG2D Extracellular Fragment - protein
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 27 - DNA, 645 bp, Homo sapiens
  misc_feature: Full Human NKG2D - DNA
gggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 28 - DNA, 405 bp, Homo sapiens
  misc_feature: Codon optimized human NKG2D fragment - DNA
ctgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtg

SEQ ID NO: 29 - DNA, 126 bp, Homo sapiens
  misc_feature: 4-1BB
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 30 - protein, 29 aa, Homo sapiens
  MISC_FEATURE: Amino Acid Sequence CD28 Transmembrane domain
FWVLVVVGGVLACYSLLVTVAFIIFWVRS

SEQ ID NO: 31 - protein, 39 aa, Homo sapiens
  MISC_FEATURE: Amino Acid Sequence CD28 IC domain
KRSRLLHSDYMNMTPRRPGPTRKHYQPYAPPRDFAAYRS

SEQ ID NO: 32 - protein, 123 aa, Homo sapiens
  MISC_FEATURE: VL Nicholson et al.
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSN

SEQ ID NO: 33 - protein, 120 aa, Homo sapiens
  MISC_FEATURE: VH Nicholson et al.
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 34 - protein, 634 aa, Homo sapiens
  MISC_FEATURE: PRT Artificial Sequence CD19R zeta chimeric receptor
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 120 aa, Homo sapiens
  MISC_FEATURE: anti-CD19 scFv HCV
EVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSS

SEQ ID NO: 36 - protein, 107 aa, Homo sapiens
  MISC_FEATURE: anti-CD19 scFv HCV
DIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIK

SEQ ID NO: 37 - protein, 11 aa, Homo sapiens
  MISC_FEATURE: LC CDR1
RASQDISKYLN

SEQ ID NO: 38 - protein, 7 aa, Homo sapiens
  MISC_FEATURE: LC CDR2
HTSRLHS

SEQ ID NO: 39 - protein, 9 aa, Homo sapiens
  MISC_FEATURE: LC CDR3
QQGNTLPYT

SEQ ID NO: 40 - protein, 10 aa, Homo sapiens
  MISC_FEATURE: HC CDR1
GVSLPDYGVS

SEQ ID NO: 41 - protein, 16 aa, Homo sapiens
  MISC_FEATURE: HC CDR2
VIWGSETTYYSSSLKS

SEQ ID NO: 42 - protein, 16 aa, Homo sapiens
  MISC_FEATURE: HC CDR2 - alternative
VIWGSETTYYQSSLKS

SEQ ID NO: 43 - protein, 16 aa, Homo sapiens
  MISC_FEATURE: HC CDR2 - alternative 2
VIWGSETTYYNSSLKS

SEQ ID NO: 44 - protein, 12 aa, Homo sapiens
  MISC_FEATURE: HC CDR3
HYYYGGSYAMDY

SEQ ID NO: 45 - protein, 107 aa, Homo sapiens
  MISC_FEATURE: Light chain variable region 1
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 46 - protein, 107 aa, Homo sapiens
  MISC_FEATURE: Light chain variable region 2
DIQMTQSPSSLSASVGDRVTITCRASQDISKYLNWYQQKPGKAVKLLIYHTSRLHSGVPSRFSGSGSGTDYTLTISSLQPEDIATYFCQQGNTLPYTFGGGTKLEIK

SEQ ID NO: 47 - protein, 107 aa, Homo sapiens
  MISC_FEATURE: Light chain variable region 3
DIQMTQSPSFLSASVGDRVTITCRASQDISKYLNWYQQKPDQAPKLLIKHTSRLHSGVPSRFSGSGSGTDFTFTISSLQPEDIATYYCQQGNTLPYTFGQGTKLEIK

SEQ ID NO: 48 - protein, 107 aa, Homo sapiens
  MISC_FEATURE: Light chain variable region 4
DIQMTQSPSTLSASVGDRVTITCRASQDISKYLNWYQQKPDQAPKLLIKHTSRLHSGVPSRFSGSGSGTDFTFTISSLQPEDIATYYCQQGNTLPYTFGQGTKLEIK

SEQ ID NO: 49 - protein, 120 aa, Homo sapiens
  MISC_FEATURE: Heavy chain variable region 1
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 50 - protein, 120 aa, Homo sapiens
  MISC_FEATURE: Heavy chain variable region 2
QVQLQESGPGLVKPSETLSVTCTVSGVSLPDYGVSWIRQPPGKGLEWLGVIWGSETTYYNSALKSRLTISKDTSKNQVFLKMSSLTAADTAIYYCAKHYYYGGSYAMDYWGQGTLVTVSS

SEQ ID NO: 51 - protein, 120 aa, Homo sapiens
  MISC_FEATURE: Heavy chain variable region 3
EVQLVESGGGLVQPGRSLRLSCTASGVSLPDYGVSWVRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 52 - protein, 120 aa, Homo sapiens
  MISC_FEATURE: Heavy chain variable region 4
QVQLQESGPGLVKPSGTLSLTCAVSGVSLPDYGVSWIRQPPGKGLEWIGVIWGSETTYYNSALKSRVTISRDNSKNTLYLQMNSLRAEDTAVYYCAKHYYYGGSYAMDYWGQGTLVTVSS

SEQ ID NO: 53 - protein, 8 aa, Homo sapiens
  MISC_FEATURE: LC CDR1
RASQDISK

SEQ ID NO: 54 - protein, 7 aa, Homo sapiens
  MISC_FEATURE: LC CDR2
IYHTSRL

SEQ ID NO: 55 - protein, 9 aa, Homo sapiens
  MISC_FEATURE: LC CDR3
QQGNTLPYT

SEQ ID NO: 56 - protein, 10 aa, Homo sapiens
  MISC_FEATURE: HC CDR1
GVSLPDYGVS

SEQ ID NO: 57 - protein, 14 aa, Homo sapiens
  MISC_FEATURE: HC CDR2
VIWGSETTYYNSAL

SEQ ID NO: 58 - protein, 12 aa, Homo sapiens
  MISC_FEATURE: HC CDR3
HYYYGGSYAMDY

SEQ ID NO: 59 - DNA, 2210 bp, Homo sapiens
  misc_feature: NK19-1b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 60 - protein, 724 aa, Homo sapiens
  MISC_FEATURE: NK19-1b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 61 - DNA, 2231 bp, Homo sapiens
  misc_feature: NK19-2b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 62 - protein, 731 aa, Homo sapiens
  MISC_FEATURE: NK19-2b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 63 - DNA, 2213 bp, Homo sapiens
  misc_feature: NK19-3b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctgttggcttacaaaaaagaagtattcatccagtgtgcacgaccctaacggtgaatacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 64 - protein, 725 aa, Homo sapiens
  MISC_FEATURE: NK19-3b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 65 - DNA, 2357 bp, Homo sapiens
  misc_feature: NK19-4b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 66 - protein, 773 aa, Homo sapiens
  MISC_FEATURE: NK19-4b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 67 - DNA, 2201 bp, Homo sapiens
  misc_feature: NK19-5b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatccattttttttctgctgcttcatcgctgtagccatgggaatccgtttcattattatggtaacacggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 68 - protein, 721 aa, Homo sapiens
  MISC_FEATURE: NK19-5b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDPFFFCCFIAVAMGIRFIIMVTRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 69 - DNA, 1805 bp, Homo sapiens
  misc_feature: NK19-6b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 70 - protein, 589 aa, Homo sapiens
  MISC_FEATURE: NK19-6b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 71 - DNA, 3347 bp, Homo sapiens
  misc_feature: NK19-7b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgcggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgcgtcgac

SEQ ID NO: 72 - protein, 1103 aa, Homo sapiens
  MISC_FEATURE: NK19-7b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYCGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 73 - DNA, 1844 bp, Homo sapiens
  misc_feature: NK19-8b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 74 - protein, 602 aa, Homo sapiens
  MISC_FEATURE: NK19-8b Protein
MALPVTALLLPLALLLHAARPDYKDDDDKGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 75 - DNA, 2243 bp, Homo sapiens
  misc_feature: NK19-9b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctcccccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 76 - protein, 735 aa, Homo sapiens
  MISC_FEATURE: NK19-9b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 77 - DNA, 2150 bp, Homo sapiens
  misc_feature: NK19-10b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcatgccggaggagggttcgggctgctcggtgcggcgcaggccctatgggtgcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 78 - protein, 704 aa, Homo sapiens
  MISC_FEATURE: NK19-10b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCMPEEGSGCSVRRRPYGCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 79 - DNA, 2234 bp, Homo sapiens
  misc_feature: NK19-11b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcatggaccaacaagcaatatatgctgagttaaacttacccacagactcaggcccagaaagttcttcaccttcatctcttcctcgggatgtctgtcagggttcaccttggcatcaatttgccctgaaacttagctgtagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 80 - protein, 732 aa, Homo sapiens
  MISC_FEATURE: NK19-11b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCMDQQAIYAELNLPTDSGPESSSPSSLPRDVCQGSPWHQFALKLSCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 81 - DNA, 2165 bp, Homo sapiens
  misc_feature: NK19-12b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcatgatcgaaacatacaaccaaacttctccccgatctgcggccactggactgcccatcagcatgaaaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 82 - protein, 709 aa, Homo sapiens
  MISC_FEATURE: NK19-12b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCMIETYNQTSPRSAATGLPISMKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 83 - DNA, 2315 bp, Homo sapiens
  misc_feature: NK19-13b DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 84 - protein, 759 aa, Homo sapiens
  MISC_FEATURE: NK19-13b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCNSRRRCGQKKKLVINSGNGAVEDRKPSGLNGEASKSQEMVHLVNKESSETPDQFMTADETRNLQNVDMKIGVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 85 - DNA, 6488 bp, Homo sapiens
  misc_feature: NK19 DNA
atgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgttaacctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcaggtagccggctaacgttaacaaccggtacctctagaactatagctagcatgcgcaaatttaaagcgctgatatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggga

SEQ ID NO: 86 - protein, 486 aa, Homo sapiens
  MISC_FEATURE: NK19 PROTEIN
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 6488 bp, Homo sapiens; identical to SEQ ID NO: 85
  misc_feature: MSCV-IRES-GFP expression plasmid

SEQ ID NO: 88 - protein, 117 aa, Homo sapiens
  MISC_FEATURE: anti CLDN6 heavy chain
EVQLQQSGPELVKPGASMKISCKASGYSFTGYTMNWVKQSHGKNLEWIGLINPYNGGTIYNQKFKGKATLTVDKSSSTAYMELLSLTSEDSAVYYCARDYGFVLDYWGQGTTLTVSS

SEQ ID NO: 89 - protein, 107 aa, Homo sapiens
  MISC_FEATURE: anti CLDN6 light chain 1
QIVLTQSPAIMSASPGEKVTITCSASSSVSYLHWFQQKPGTSPKLWVYSTSNLPSGVPARFGGSGSGTSYSLTISRMEAEDAATYYCQQRSIYPPWTFGGGTKLEIK

SEQ ID NO: 90 - protein, 107 aa, Homo sapiens
  MISC_FEATURE: anti CLDN6 light chain 2
QIVLTQSPSIMSVSPGEKVTITCSASSSVSYMHWFQQKPGTSPKLGIYSTSNLASGVPARFSGRGSGTSYSLTISRVAAEDAATYYCQQRSNYPPWTFGGGTKLEIK

SEQ ID NO: 91 - protein, 107 aa, Homo sapiens
  MISC_FEATURE: anti CLDN6 light chain 3
QIVLTQSPSIMSVSPGEKVTITCSASSSVSYMHWFQQKPGTSPKLSIYSTSNLASGVPARFSGRGSGTSYSLTISRVAAEDAATYYCQQRSNYPPWTFGGGTKLEIK

SEQ ID NO: 92 - protein, 8 aa, Homo sapiens
  MISC_FEATURE: anti CLDN6 heavy chain CDR1
GYSFTGYT

SEQ ID NO: 93 - protein, 8 aa, Homo sapiens
  MISC_FEATURE: anti CLDN6 heavy chain CDR2
INPYNGGT

SEQ ID NO: 94 - protein, 10 aa, Homo sapiens
  MISC_FEATURE: anti CLDN6 heavy chain CDR3
ARDYGFVLDY

SEQ ID NO: 95 - protein, 5 aa, Homo sapiens
  MISC_FEATURE: anti CLDN6 light chain 1 CDR1
SSVSY

SEQ ID NO: 96 - protein, 3 aa, Homo sapiens
  MISC_FEATURE: anti CLDN6 light chain 1  CDR2
STS

SEQ ID NO: 97 - protein, 8 aa, Homo sapiens
  MISC_FEATURE: anti CLDN6 light chain 1 CDR3
QQRSIYPP

SEQ ID NO: 98 - protein, 5 aa, Homo sapiens
  MISC_FEATURE: anti CLDN6 light chain 2 CDR1
SSVSY

SEQ ID NO: 99 - protein, 3 aa, Homo sapiens
  MISC_FEATURE: anti CLDN6 light chain 2  CDR2
STS

SEQ ID NO: 100 - protein, 8 aa, Homo sapiens
  MISC_FEATURE: anti CLDN6 light chain 2 CDR3
QQRSNYPP

SEQ ID NO: 101 - protein, 5 aa, Homo sapiens
  MISC_FEATURE: anti CLDN6 light chain 3 CDR1
SSVSY

SEQ ID NO: 102 - protein, 3 aa, Homo sapiens
  MISC_FEATURE: anti CLDN6 light chain 3  CDR2
STS

SEQ ID NO: 103 - protein, 8 aa, Homo sapiens
  MISC_FEATURE: anti CLDN6 light chain 3 CDR3
QQRSNYPP

SEQ ID NO: 104 - protein, 120 aa, Homo sapiens
  MISC_FEATURE: anti CD19 heavy chain variable region
QVQLVQSGGGVVQPGRSLRLSCAASGVSLPDYGVSWVRQAPGKGLEWVAVIWGSETTYYNSALKSRFTISRDNSKNTLYLQMNSLRAEDTAVYYCAKHYYYGGSYAMDYWGQGTLVTVSS

SEQ ID NO: 105 - protein, 107 aa, Homo sapiens
  MISC_FEATURE: anti CD19 light chain variable region
DIQMTQSPSSLSASVGDRVTITCRASQDISKYLNWYQQKPGKAIKLLIYHTSRLHSGVPSRFSGSGSGTDFTLTISSLQPEDFATYYCQQGATLPYTFGPGTKVDIK

SEQ ID NO: 106 - protein, 450 aa, Homo sapiens
  MISC_FEATURE: anti CD19 heavy chain variable
QVQLVQSGGGVVQPGRSLRLSCAASGVSLPDYGVSWVRQAPGKGLEWVAVIWGSETTYYNSALKSRFTISRDNSKNTLYLQMNSLRAEDTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 214 aa, Homo sapiens
  MISC_FEATURE: anti CD19 light chain variable
DIQMTQSPSSLSASVGDRVTITCRASQDISKYLNWYQQKPGKAIKLLIYHTSRLHSGVPSRFSGSGSGTDFTLTISSLQPEDFATYYCQQGATLPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 11 aa, Homo sapiens
  MISC_FEATURE: anti CD19 LCCDR1
RASQDISKYLN

SEQ ID NO: 109 - protein, 7 aa, Homo sapiens
  MISC_FEATURE: anti CD19 LCCDR2
HTSRLHS

SEQ ID NO: 110 - protein, 9 aa, Homo sapiens
  MISC_FEATURE: anti CD19 LCCDR3
QQGNTLPYT

SEQ ID NO: 111 - protein, 10 aa, Homo sapiens
  MISC_FEATURE: anti CD19 HCCDR1
GVSLPDYGVS

SEQ ID NO: 112 - protein, 16 aa, Homo sapiens
  MISC_FEATURE: anti CD19 HCCDR2
VIWGSETTYYSSSLKS

SEQ ID NO: 113 - protein, 16 aa, Homo sapiens
  MISC_FEATURE: anti CD19 HCCDR2
VIWGSETTYYQSSLKS

SEQ ID NO: 114 - protein, 16 aa, Homo sapiens
  MISC_FEATURE: anti CD19 HCCDR2
VIWGSETTYYNSSLKS

SEQ ID NO: 115 - protein, 12 aa, Homo sapiens
  MISC_FEATURE: anti CD19 HCCDR3
HYYYGGSYAMDY

SEQ ID NO: 116 - protein, 242 aa, Homo sapiens
  MISC_FEATURE: antiCD19 CAR
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 117 - protein, 107 aa, Homo sapiens
  MISC_FEATURE: huFMC63VLv1
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEIT

SEQ ID NO: 118 - protein, 107 aa, Homo sapiens
  MISC_FEATURE: HUFMC63VLV2
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEIT

SEQ ID NO: 119 - protein, 107 aa, Homo sapiens
  MISC_FEATURE: HUFMC63VLV3
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEIT

SEQ ID NO: 120 - protein, 120 aa, Homo sapiens
  MISC_FEATURE: HUFMC63VHV1
QVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSS

SEQ ID NO: 121 - protein, 120 aa, Homo sapiens
  MISC_FEATURE: HUFMC63VHV2
QVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSS

SEQ ID NO: 122 - protein, 120 aa, Homo sapiens
  MISC_FEATURE: HUFMC63VHV3
QVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSS

SEQ ID NO: 123 - protein, 120 aa, Homo sapiens
  MISC_FEATURE: HUFMC63VHV4
QVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSS

SEQ ID NO: 124 - protein, 11 aa, Homo sapiens
  MISC_FEATURE: huFMC63VLv1 CDR1
RASQDISKYLN

SEQ ID NO: 125 - protein, 7 aa, Homo sapiens
  MISC_FEATURE: huFMC63VLv1 CDR2
HTSRLHS

SEQ ID NO: 126 - protein, 9 aa, Homo sapiens
  MISC_FEATURE: huFMC63VLv1 CDR3
QQGNTLPYT

SEQ ID NO: 127 - protein, 11 aa, Homo sapiens
  MISC_FEATURE: HUFMC63VLV2 CDR1
RASQDISKYLN

SEQ ID NO: 128 - protein, 7 aa, Homo sapiens
  MISC_FEATURE: HUFMC63VLV2 CDR2
HTSRLHS

SEQ ID NO: 129 - protein, 9 aa, Homo sapiens
  MISC_FEATURE: HUFMC63VLV2 CDR3
QQGNTLPYT

SEQ ID NO: 130 - protein, 11 aa, Homo sapiens
  MISC_FEATURE: HUFMC63VLV3 CDR1
RASQDISKYLN

SEQ ID NO: 131 - protein, 7 aa, Homo sapiens
  MISC_FEATURE: HUFMC63VLV3 CDR2
HTSRLHS

SEQ ID NO: 132 - protein, 9 aa, Homo sapiens
  MISC_FEATURE: HUFMC63VLV3 CDR3
QQGNTLPYT

SEQ ID NO: 133 - protein, 10 aa, Homo sapiens
  MISC_FEATURE: HUFMC63VHV1 CDR1
GVSLPDYGVS

SEQ ID NO: 134 - protein, 16 aa, Homo sapiens
  MISC_FEATURE: HUFMC63VHV1 CDR2
VIWGSETTYYNSALKS

SEQ ID NO: 135 - protein, 12 aa, Homo sapiens
  MISC_FEATURE: HUFMC63VHV1 CDR3
HYYYGGSYAMDY

SEQ ID NO: 136 - protein, 10 aa, Homo sapiens
  MISC_FEATURE: HUFMC63VHV2 CDR1
GVSLPDYGVS

SEQ ID NO: 137 - protein, 16 aa, Homo sapiens
  MISC_FEATURE: HUFMC63VHV2 CDR2
VIWGSETTYYNSALKS

SEQ ID NO: 138 - protein, 12 aa, Homo sapiens
  MISC_FEATURE: HUFMC63VHV2 CDR3
HYYYGGSYAMDY

SEQ ID NO: 139 - protein, 10 aa, Homo sapiens
  MISC_FEATURE: HUFMC63VHV3 CDR1
GVSLPDYGVS

SEQ ID NO: 140 - protein, 16 aa, Homo sapiens
  MISC_FEATURE: HUFMC63VHV3 CDR2
VIWGSETTYYNSALKS

SEQ ID NO: 141 - protein, 12 aa, Homo sapiens
  MISC_FEATURE: HUFMC63VHV3 CDR3
HYYYGGSYAMDY

SEQ ID NO: 142 - protein, 10 aa, Homo sapiens
  MISC_FEATURE: HUFMC63VHV4 CDR1
GVSLPDYGVS

SEQ ID NO: 143 - protein, 16 aa, Homo sapiens
  MISC_FEATURE: HUFMC63VHV4 CDR2
VIWGSETTYYNSALKS

SEQ ID NO: 144 - protein, 12 aa, Homo sapiens
  MISC_FEATURE: HUFMC63VHV4 CDR3
HYYYGGSYAMDY

SEQ ID NO: 145 - DNA, 1122 bp, Homo sapiens
  misc_feature: DNA Sequence  NKG2D-CD8a hinge-Ox40-CD3z
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 146 - protein, 341 aa, Homo sapiens
  MISC_FEATURE: Amino Acid Sequence NKG2D-IgG4 hinge-OX40-CD3z
  MISC_FEATURE: Amino Acid Sequence NKG2D-***IgG4 hinge***- OX40-CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVESKYGPPCPSCPIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - DNA, 20 bp, Homo sapiens
  misc_feature: TGFBR2 guide RNA 1
cccctaccatgactttattc

SEQ ID NO: 148 - DNA, 20 bp, Homo sapiens
  misc_feature: TGFBR2 guide RNA 2
attgcactcatcagagctac

SEQ ID NO: 149 - DNA, 20 bp, Homo sapiens
  misc_feature: TGFBR2 guide RNA 3
agtcatggtaggggagcttg

SEQ ID NO: 150 - DNA, 20 bp, Homo sapiens
  misc_feature: TGFBR2 guide RNA 4
tgctggcgatacgcgtccac

SEQ ID NO: 151 - DNA, 20 bp, Homo sapiens
  misc_feature: TGFBR2 guide RNA 5
gtgagcaatcccccgggcga

SEQ ID NO: 152 - DNA, 20 bp, Homo sapiens
  misc_feature: TGFBR2 guide RNA 6
aacgtgcggtgggatcgtgc

SEQ ID NO: 153 - DNA, 20 bp, Homo sapiens
  misc_feature: CISH guide RNA 1
ctcaccagattcccgaaggt

SEQ ID NO: 154 - DNA, 20 bp, Homo sapiens
  misc_feature: CISH guide RNA 2
ccgccttgtcatcaaccgtc

SEQ ID NO: 155 - DNA, 20 bp, Homo sapiens
  misc_feature: CISH guide RNA 3
tctgcgttcaggggtaagcg

SEQ ID NO: 156 - DNA, 20 bp, Homo sapiens
  misc_feature: CISH guide RNA 4
gcgcttacccctgaacgcag

SEQ ID NO: 157 - DNA, 20 bp, Homo sapiens
  misc_feature: CISH guide RNA 5
cgcagaggaccatgtccccg

SEQ ID NO: 158 - DNA, 20 bp, Homo sapiens
  misc_feature: NKG2A Guide RNA 1
ggagctgatggtaaatctgc

SEQ ID NO: 159 - DNA, 20 bp, Homo sapiens
  misc_feature: NKG2A guide RNA 2
ttgaaggtttaattccgcat

SEQ ID NO: 160 - DNA, 20 bp, Homo sapiens
  misc_feature: NKG2A guide RNA 3
aacaactatcgttaccacag

SEQ ID NO: 161 - DNA, 20 bp, Homo sapiens
  misc_feature: CD70 guide RNA 1
tcaccaagcccgcgaccaat

SEQ ID NO: 162 - DNA, 20 bp, Homo sapiens
  misc_feature: CD70 guide RNA 2
gctttggtcccattggtcgc

SEQ ID NO: 163 - DNA, 20 bp, Homo sapiens
  misc_feature: CD70 guide RNA 3
accctcctccggcatcgccg

SEQ ID NO: 164 - DNA, 23 bp, Homo sapiens
  misc_feature: CBLB guide RNA 1
taatctggtggacctcatgaagg

SEQ ID NO: 165 - DNA, 23 bp, Homo sapiens
  misc_feature: CBLB guide RNA 2
tcggttggcaaacgtccgaaagg

SEQ ID NO: 166 - DNA, 23 bp, Homo sapiens
  misc_feature: CBLB guide RNA 3
agcaagctgccgcagatcgcagg

SEQ ID NO: 167 - DNA, 23 bp, Homo sapiens
  misc_feature: TRIM29 guide RNA 1
gaacggtaggtcccctctcgtgg

SEQ ID NO: 168 - DNA, 23 bp, Homo sapiens
  misc_feature: TRIM29 guide RNA 2
agctgccttggacgacgggcagg

SEQ ID NO: 169 - DNA, 23 bp, Homo sapiens
  misc_feature: TRIM29 guide RNA 3
tgagccgtaacttcattgagagg

SEQ ID NO: 170 - DNA, 20 bp, Homo sapiens
  misc_feature: CD45 guide RNA 1
agtgctggtgttgggcgcac

SEQ ID NO: 171 - DNA, 23 bp, Homo sapiens
  misc_feature: SOCS2 guide RNA 1
gtgaacagtgccgttccgggggg

SEQ ID NO: 172 - DNA, 23 bp, Homo sapiens
  misc_feature: SOCS2-2 gRNA
ggcaccggtacatttgttaatgg

SEQ ID NO: 173 - DNA, 23 bp, Homo sapiens
  misc_feature: SOCS2-3 gRNA
ttcgccagacgcgccgcctgcgg

SEQ ID NO: 174 - protein, 374 aa, Homo sapiens
  MISC_FEATURE: NKG2D-CD8ahinge/tm-OX40-CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - DNA, 2150 bp, Homo sapiens
  misc_feature: DNA NK19H-NF-1
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 176 - protein, 706 aa, Homo sapiens
  MISC_FEATURE: AA NK19H-NF-1
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 177 - DNA, 2150 bp, Homo sapiens
  misc_feature: DNA NK19H-NF-2
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 178 - protein, 706 aa, Homo sapiens
  MISC_FEATURE: AA NK19H-NF-2
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 179 - DNA, 2150 bp, Homo sapiens
  misc_feature: DNA NK19H-NF-3
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 180 - protein, 706 aa, Homo sapiens
  MISC_FEATURE: AA NK19H-NF-3
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 181 - DNA, 2059 bp, Homo sapiens
  misc_feature: DNA NK19H-NF-4
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggctgggcgtgatttggggcagcgaaaccacctattataacagcgcgctgaaaagccgcctgaccattagcaaagataacagcaaaagccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcg

SEQ ID NO: 182 - protein, 706 aa, Homo sapiens
  MISC_FEATURE: AA NK19H-NF-4
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 183 - DNA, 2153 bp, Homo sapiens
  misc_feature: DNA NK19H-NF-5
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcagatctagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 184 - protein, 707 aa, Homo sapiens
  MISC_FEATURE: AA NK19H-NF-5
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 185 - DNA, 2150 bp, Homo sapiens
  misc_feature: DNA NK19H-NF-6
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 186 - protein, 706 aa, Homo sapiens
  MISC_FEATURE: AA NK19H-NF-6
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 187 - DNA, 2150 bp, Homo sapiens
  misc_feature: DNA NK19H-NF-7
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttactactgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 188 - protein, 706 aa, Homo sapiens
  MISC_FEATURE: AA NK19H-NF-7
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 189 - DNA, 2150 bp, Homo sapiens
  misc_feature: DNA NK19H-NF-8
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 190 - protein, 706 aa, Homo sapiens
  MISC_FEATURE: AA NK19H-NF-8
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 191 - DNA, 2150 bp, Homo sapiens
  misc_feature: DNA NK19H-NF-9
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 192 - protein, 706 aa, Homo sapiens
  MISC_FEATURE: AA NK19H-NF-9
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 193 - DNA, 2150 bp, Homo sapiens
  misc_feature: DNA NK19H-NF-10
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 194 - protein, 706 aa, Homo sapiens
  MISC_FEATURE: AA NK19H-NF-10
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 195 - DNA, 2150 bp, Homo sapiens
  misc_feature: DNA NK19H-NF-11
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 196 - protein, 706 aa, Homo sapiens
  MISC_FEATURE: AA NK19H-NF-11
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 197 - DNA, 2150 bp, Homo sapiens
  misc_feature: DNA NK19H-NF-12
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 198 - protein, 706 aa, Homo sapiens
  MISC_FEATURE: AA NK19H-NF-12
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

(19) United States
(12) Patent Application Publication
(10) Pub. No.: US 2022/0233593 A1
(43) Pub. Date: Jul. 28, 2022

(54) COMBINATIONS OF ENGINEERED NATURAL KILLER CELLS AND ENGINEERED T CELLS FOR IMMUNOTHERAPY

(71) Applicant: Nkarta, Inc., South San Francisco, CA (US)

(72) Inventors: James Barnaby Trager, Albany, CA (US); Luxuan Guo Buren, San Francisco, CA (US); Chao Guo, San Francisco, CA (US); Guangnan Li, Foster City, CA (US); Daofeng Liu, Pleasanton, CA (US); Ivan Chan, Millbrae, CA (US)

(21) Appl. No.: 17/596,166

(22) PCT Filed: Jun. 2, 2020
(86) PCT No.: PCT/US2020/035752

§ 371 Date: Dec. 3, 2021

Related U.S. Application Data
(60) Provisional application No. 62/857,167, filed on Jun. 4, 2019.
(60) Provisional application No. 62/943,697, filed on Dec. 4, 2019.

(51) Int. Cl. A61K 35/17; C12N 5/0783; A61K 38/20; A61K 38/17

(52) U.S. Cl. CPC A61K 35/17; C12N 5/0646; C12N 2510/00; A61K 38/2086; A61K 38/1774; C12N 5/0636

(57) ABSTRACT

Several embodiments of the methods and compositions disclosed herein relate to immune cells that are engineered to express chimeric antigen receptors and/or genetically modified to enhance one or more aspects of the efficacy of the immune cells in cellular immunotherapy. Several embodiments relate to genetic modifications which reduce potential side effects of cellular immunotherapy. In several embodiments, combinations of cells are used to achieve both rapid and long-term tumor reduction with reduced or eliminated potential for graft versus host effects.

### RELATED CASES

This application claims the benefit of priority of U.S. Provisional Patent Application No. 62/857,167, filed Jun. 4, 2019 and U.S. Provisional Patent Application No. 62/943,697, filed Dec. 4, 2019, the entire contents of each of which is incorporated by reference herein.

### FIELD

Several embodiments disclosed herein relate to methods and compositions comprising genetically engineered cells for cancer immunotherapy, in particular combinations of engineered immune cell types. In several embodiments, the present disclosure relates to cells engineered to express chimeric antigen receptors. In several embodiments, further engineering is performed to enhance the efficacy and/or reduce potential side effects when the cells are used in cancer immunotherapy.

### BACKGROUND

As further knowledge is gained about various cancers and what characteristics a cancerous cell has that can be used to specifically distinguish that cell from a healthy cell, therapeutics are under development that leverage the distinct features of a cancerous cell. Immunotherapies that employ engineered immune cells are one approach to treating cancers.

### INCORPORATION BY REFERENCE OF MATERIAL IN ASCII TEXT FILE

This application incorporates by reference the Sequence Listing contained in the following ASCII text file being submitted concurrently herewith: File name: NKT043WO_ST25.txt; created Jun. 1, 2020, 327 KB in size.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of chimeric receptors in immune cells to achieve the targeted recognition and destruction of aberrant cells of interest.

In several embodiments, cells for immunotherapy are genetically modified to enhance one or more characteristics of the cells that results in a more effective therapeutic. In several embodiments, one or more of the expansion potential, cytotoxicity and/or persistence of the genetically modified immune cells is enhanced. In several embodiments, the immune cells are also engineered to express a cytotoxic receptor that targets a tumor. There is provided for herein, in several embodiments, a population of genetically engineered natural killer (NK) cell for cancer immunotherapy, comprising a plurality of NK cells, wherein the plurality of NK cells are engineered to express a cytotoxic receptor comprising an extracellular ligand binding domain, a transmembrane domain, and a cytotoxic signaling complex, wherein the NK cells are genetically edited to express reduced levels of a cytokine-inducible SH2-containing (CIS) protein encoded by a CISH gene as compared to a non-engineered NK cell, wherein the reduced CIS expression was engineered through editing of a CISH gene, and wherein the genetically engineered NK cells exhibit one or more of enhanced expansion capability, enhanced cytotoxicity against target cells, and enhanced persistence, as compared to NK cells expressing native levels of CIS. In several embodiments, the cytotoxic signaling complex comprises an OX-40 subdomain and a CD3zeta subdomain. In several embodiments, the NK cells are engineered to express membrane bound IL-15. In several embodiments, T cells are engineered and used in place of, or in addition to NK cells. In several embodiments, NKT cells are not included in the engineered immune cell population. In several embodiments, the population of immune cells comprises, consists of, or consists essentially of engineered NK cells.

In several embodiments, the extracellular ligand binding domain comprises a receptor that is directed against a tumor marker selected from the group consisting of MICA, MICB, ULBP1, ULBP2, ULBP3, ULBP4, ULBP5, and ULBP6. In several embodiments, the cytotoxic receptor expressed by the NK cells comprises, consists of, or consists essentially of (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the cytotoxic receptor is encoded by a polynucleotide having at least 85%, 90%, 95%, 96%, 97%, 98%, or 99% sequence identity to SEQ ID NO: 145. In several embodiments, the cytotoxic receptor has at least 85%, 90%, 95%, 96%, 97%, 98%, or 99% sequence identity to SEQ ID NO: 174.

In several embodiments, the cytotoxic receptor expressed by the NK cells comprises a chimeric antigen receptor (CAR) that comprises, consists of, or consists essentially of (i) an tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain. In several embodiments, the anti-CD19 antibody comprises a variable heavy (VH) domain of a single chain Fragment variable (scFv) and a variable light (VL) domain of a scFv, wherein the VH domain comprises the amino acid sequence of SEQ ID NO: 120, and wherein the encoded VL domain comprises the amino acid sequence of SEQ ID NO: 118. In several embodiments, the CAR expressed by the T cells has at least 95% sequence identity to the amino acid sequence set forth in SEQ ID NO: 178. In several embodiments, the anti-CD19 antibody fragment is designed (e.g., engineered) to reduce potential antigenicity of the encoded protein and/or enhance one or more characteristics of the encoded protein (e.g., target recognition and/or binding characteristics) Thus, according to several embodiments, the anti-CD19 antibody fragment does not comprise certain sequences. For example, according to several embodiments the anti-CD19 antibody fragment is not encoded by SEQ ID NO: 116, nor does it comprise the VL regions of SEQ ID NO: 105 or 107, or the VH regions of SEQ ID NO: 104 or 106. In several embodiments, the anti-CD19 antibody fragment does not comprise one or more CDRs selected from SEQ ID NO: 108 to 115.

In several embodiments, the expression of CIS is substantially reduced as compared to a non-engineered NK cell. According to certain embodiments provided for herein, gene editing can reduce expression of a target protein, like CIS (or others disclosed herein) by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. Thus, in several embodiments, immune cells (e.g., NK cells) do not express a detectable level of CIS protein.

In several embodiments, the NK cells are further genetically engineered to express a reduced level of a transforming growth factor beta receptor (TGFBR) as compared to a non-engineered NK cell. In several embodiments, at least 50% of the population of NK cells do not express a detectable level of the TGFBR. In several embodiments, the NK cells are further genetically edited to express a reduced level of beta-2 microgolublin (B2M) as compared to a non-engineered NK cell. In several embodiments, at least 50% of the population of NK cells do not express a detectable level of B2M surface protein. In several embodiments, the NK cells are further genetically edited to express a reduced level of CIITA (class II major histocompatibility complex transactivator) as compared to a non-engineered NK cell. In several embodiments, at least 50% of the population of NK cells do not express a detectable level of CIITA. In several embodiments, the NK cells are further genetically edited to express a reduced level of a Natural Killer Group 2, member A (NKG2A) receptor as compared to a non-engineered NK cell. In several embodiments, at least 50% of the population of NK cells do not express a detectable level of NKG2A. In several embodiments, the NK cells are further genetically edited to express a reduced level of a Cbl proto-oncogene B protein encoded by a CBLB gene as compared to a non-engineered NK cell. In several embodiments, at least 50% of the population of NK cells do not express a detectable level of Cbl proto-oncogene B protein. In several embodiments, the NK cells are further genetically edited to express a reduced level of a tripartite motif-containing protein 29 protein encoded by a TRIM29 gene as compared to a non-engineered NK cell. In several embodiments, at least 50% of the population of NK cells do not express a detectable level of TRIM29 protein. In several embodiments, the NK cells are further genetically edited to express a reduced level of a suppressor of cytokine signaling 2 protein encoded by a SOCS2 gene as compared to a non-engineered NK cell. In several embodiments, at least 50% of the population of NK cells do not express a detectable level of SOCS2 protein. Depending on the embodiment, any combination of the above-referenced target proteins/genes can be edited to a desired level, including in combination with CIS, including such that the proteins are not expressed at a detectable level. In several embodiments, there may remain some amount of protein that is detectable, but the function of the protein is disrupted, substantially disrupted, eliminated or substantially eliminated. In several embodiments, even if some functionality remains, the positive effects imparted to the engineered immune cell (e.g., NK cell or T cell) remain and serve to enhance one or more anti-cancer aspects of the cells.

In several embodiments, the NK cells are further genetically edited to disrupt expression of at least one immune checkpoint protein by the NK cells. In several embodiments, the at least one immune checkpoint protein is selected from CTLA4, PD-1, lymphocyte activation gene (LAG-3), NKG2A receptor, KIR2DL-1, KIR2DL-2, KIR2DL-3, KIR2DS-1 and/or KIR2DA-2, and combinations thereof.

In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed). For example in several embodiments, the NK cells are further genetically edited to express CD47. In several embodiments, the NK cells are further genetically engineered to express HLA-E. Any genes that are knocked in can be knocked in in combination with any of the genes that are knocked out or otherwise disrupted.

In several embodiments, the population of genetically engineered NK cells further comprises a population of genetically engineered T cells. In several embodiments, the population of T cells is at least partially, if not substantially, non-alloreactive. In several embodiments, the non-alloreactive T cells comprise at least one genetically edited subunit of a T Cell Receptor (TCR) such that the non-alloreactive T cells do not exhibit alloreactive effects against cells of a recipient subject. In several embodiments, the population of T cells is engineered to express a chimeric antigen receptor (CAR) directed against a tumor marker, wherein the tumor marker is one or more of CD19, CD123, CD70, Her2, mesothelin, Claudin 6, BCMA, PD-L1, EGFR. Combinations of two or more of these tumor markers can be targeted, in some embodiments. In several embodiments, the CAR expressed by the T cells is directed against CD19. In several embodiments, the CAR expressed by the T cells has at least 85%, 90%, 95%, 96%, 97%, 98%, or 99% sequence identity to the amino acid sequence set forth in SEQ ID NO: 178. In several embodiments, the CAR targets CD19. In several embodiments, the CAR is designed (e.g., engineered) to reduce potential antigenicity of the encoded protein and/or enhance one or more characteristics of the encoded protein (e.g., target recognition and/or binding characteristics) Thus, according to several embodiments, anti-CD19 CAR does not comprise certain sequences. For example, according to several embodiments the anti-CD19 CAR does not comprise by SEQ ID NO: 116, SEQ ID NO: 105, 107, 104 or 106. In several embodiments, the anti-CD19 antibody fragment does not comprise one or more CDRs selected from SEQ ID NO: 108 to 115.

In several embodiments, the TCR subunit of the T cells modified is TCRα. In several embodiments, the modification to the TCR of the T cells results in at least 80%, 85%, or 90% of the population of T cells not expressing a detectable level of the TCR. As with the edited NK cells disclosed herein, in several embodiments, the T cells are further genetically edited to reduce expression of one or more of CIS, TGFBR, B2M, CIITA, TRIM29 and SOCS2 as compared to non-engineered T cells, or to express CD47 or HLA-E. In several embodiments, the T cells are further genetically edited to disrupt expression of at least one immune checkpoint protein by the T cells, wherein the at least one immune checkpoint protein is selected from CTLA4, PD-1, and lymphocyte activation gene (LAG-3).

Depending on the embodiment, the gene editing of the NK cells and/or the T cells in order to reduce expression and/or the gene editing to induce expression is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, and combinations thereof. In several embodiments, the Cas is Cas9. In several embodiments, the CRISPR-Cas system comprises a Cas selected from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, the gene editing of the NK cells and/or the T cells in order to reduce expression and/or the gene editing to induce expression is made using a zinc finger nuclease (ZFN). In several embodiments, the gene editing of the NK cells and/or the T cells in order to reduce expression and/or the gene editing to induce expression is made using a Transcription activator-like effector nuclease (TALEN).

In several embodiments, the genetically engineered NK cells and/or engineered T cells have an OX40 subdomain encoded by a sequence having at least 85%, 90%, or 95% sequence identity to SEQ ID NO. 5. In several embodiments, the genetically engineered NK cells and/or genetically engineered T cells have a CD3 zeta subdomain encoded by a sequence having at least 85%, 90%, or 95% sequence identity to SEQ ID NO. 7. In several embodiments, the genetically engineered NK cells and/or genetically engineered T cells have an mbIL15 encoded by a sequence having at least 85%, 90%, or 95% sequence identity to SEQ ID NO. 11.

Also provided for herein are methods of treating cancer in a subject, comprising administering to the subject a population of genetically engineered NK cells (and/or a population of genetically engineered T cells) as disclosed herein. Provided for herein is also a use of the population of genetically engineered NK cells (and/or a population of genetically engineered T cells) as disclosed herein in the treatment of cancer. Provided for herein is also a use of the population of genetically engineered NK cells (and/or a population of genetically engineered T cells) as disclosed herein in the manufacture of a medicament for the treatment of cancer.

Methods of treating cancer are also provided for herein. In several embodiments, there is provided a method for treating cancer in a subject comprising administering to the subject a population of genetically engineered immune cells, comprising (i) a plurality of NK cells, wherein the plurality of NK cells are engineered to express a cytotoxic receptor comprising an extracellular ligand binding domain, a transmembrane domain, and a cytotoxic signaling complex, wherein the NK cells are genetically edited to express reduced levels of cytokine-inducible SH2-containing (CIS) protein encoded by a CISH gene by the cells as compared to a non-engineered NK cell, wherein the reduced CIS expression was engineered through genetic editing of a CISH gene, and wherein the genetically engineered NK cells exhibit one or more of enhanced expansion capability, enhanced cytotoxicity against target cells, and enhanced persistence, as compared to NK cells expressing native levels of CIS; and optionally (ii) a plurality of T cells.

In several embodiments, the cytotoxic signaling complex comprises an OX-40 subdomain and a CD3zeta subdomain. In several embodiments, the NK cells are also engineered to express membrane bound IL-15.

In several embodiments, when included, the plurality of T cells are substantially non-alloreactive. Advantageously, in several embodiments, the non-alloreactive T cells comprise at least one modification to a subunit of a T Cell Receptor (TCR) such that the non-alloreactive T cells do not exhibit alloreactive effects against cells of a recipient subject. In several embodiments, the T cells are also engineered to express a chimeric antigen receptor (CAR) directed against a tumor marker, which can be selected from CD19, CD123, CD70, Her2, mesothelin, Claudin 6, BCMA, PD-L1, EGFR, and combinations thereof.

In several embodiments, the cytotoxic receptor expressed by the NK cells comprises (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the cytotoxic receptor is encoded by a polynucleotide having at least 80%, 85%, 90%, or 95% sequence identity to SEQ ID NO: 145. In several embodiments, the cytotoxic receptor has at least 80%, 85%, 90%, or 95% sequence identity to SEQ ID NO: 174. In several embodiments, the cytotoxic receptor expressed by the NK cells is directed against CD19. In several embodiments, the cytotoxic receptor expressed by the NK cells has at least 80%, 85%, 90%, or 95% sequence identity to the amino acid sequence set forth in SEQ ID NO: 178. In several embodiments, the CAR expressed by the T cells is directed against CD19. In several embodiments, the CAR expressed by the T cells (and or the NK cells) comprises (i) an tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain. In several embodiments, the polynucleotide encoding the CAR also encodes for membrane bound IL15. In several embodiments, the anti-CD19 antibody fragment comprises a variable heavy (VH) domain of a single chain Fragment variable (scFv) and a variable light (VL) domain of a scFv. In several embodiments, the VH domain comprises the amino acid sequence of SEQ ID NO: 120 and wherein the VL domain comprises the amino acid sequence of SEQ ID NO: 118.

In several embodiments, the NK cells and/or the T cells are further genetically edited to reduce expression of one or more of CIS, TGFBR, B2M, CIITA, TRIM29 and SOCS2 as compared to a non-engineered T cells, or to express CD47 or HLA-E.

In several embodiments, the NK cells and/or the T cells are further genetically edited to disrupt expression of at least one immune checkpoint protein by the cells, wherein the at least one immune checkpoint protein is selected from CTLA4, PD-1, and lymphocyte activation gene (LAG-3), NKG2A receptor, KIR2DL-1, KIR2DL-2, KIR2DL-3, KIR2DS-1 and/or KIR2DA-2.

In several embodiments, the OX40 subdomain is encoded by a sequence having at least 80%, 85%, 90%, or 95% sequence identity to SEQ ID NO. 5. In several embodiments, the CD3 zeta subdomain is encoded by a sequence having at least 80%, 85%, 90%, or 95% sequence identity to SEQ ID NO. 7. In several embodiments, mbIL15 is encoded by a sequence having at least 80%, 85%, 90%, or 95% sequence identity to SEQ ID NO. 11.

Depending on the embodiment of the methods disclosed herein that are applied, the gene editing of the NK cells and/or the T cells in order to reduce expression and/or the gene editing to induce expression is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, and combinations thereof. In several embodiments, the Cas is Cas9. In several embodiments, the CRISPR-Cas system comprises a Cas selected from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, the gene editing of the NK cells and/or the T cells in order to reduce expression and/or the gene editing to induce expression is made using a zinc finger nuclease (ZFN). In several embodiments, the gene editing of the NK cells and/or the T cells in order to reduce expression and/or the gene editing to induce expression is made using a Transcription activator-like effector nuclease (TALEN).

Additionally provided for herein is a mixed population of engineered immune cells for cancer immunotherapy, comprising a plurality of NK cells, wherein the plurality of NK cells are engineered to express a cytotoxic receptor comprising an extracellular ligand binding domain, a transmembrane domain, and a cytotoxic signaling complex, wherein the NK cells are genetically edited to express reduced levels of cytokine-inducible SH2-containing (CIS) protein encoded by a CISH gene by the cells as compared to a non-engineered NK cell, wherein the reduced CIS expression was engineered through genetic editing of a CISH gene, and wherein the genetically engineered NK cells exhibit one or more of enhanced expansion capability, enhanced cytotoxicity against target cells, and enhanced persistence, as compared to NK cells expressing native levels of CIS, and a plurality of T cells that are substantially non-alloreactive through at least one modification to a subunit of a T Cell Receptor (TCR), wherein the population of T cells is engineered to express a chimeric antigen receptor (CAR) directed against a tumor marker selected from one or more of CD19, CD123, CD70, Her2, mesothelin, Claudin 6, BCMA, PD-L1, and EGFR. In several embodiments, the cytotoxic signaling complex of the cytotoxic receptor and/or CAR comprises an OX-40 subdomain and a CD3zeta subdomain. In several embodiments, the NK cells and/or the T cells are engineered to express membrane bound IL-15. In several embodiments, the cytotoxic receptor expressed by the NK cells has at least 80%, 85%, 90%, or 95% sequence identity to SEQ ID NO: 174. In several embodiments, the cytotoxic receptor expressed by the NK cells has at least 80%, 85%, 90%, or 95% sequence identity to the amino acid sequence set forth in SEQ ID NO: 178. In several embodiments, the CAR expressed by the T cells has at least 80%, 85%, 90%, or 95% sequence identity to the amino acid sequence set forth in SEQ ID NO: 178.

Provided for herein, in several embodiments, is a population of genetically altered immune cells for cancer immunotherapy, comprising a population of immune cells that are genetically modified to reduce the expression of a cytokine-inducible SH2-containing protein encoded by a CISH gene by the immune cell, genetically modified to reduce the expression of a transforming growth factor beta receptor by the immune cell, genetically modified to reduce the expression of a Natural Killer Group 2, member A (NKG2A) receptor by the immune cell, genetically modified to reduce the expression of a Cbl proto-oncogene B protein encoded by a CBLB gene by the immune cell, genetically modified to reduce the expression of a tripartite motif-containing protein 29 protein encoded by a TRIM29 gene by the immune cell, and/or genetically modified to reduce the expression of a suppressor of cytokine signaling 2 protein encoded by a SOCS2 gene by the immune cell, and genetically engineered to express a chimeric antigen receptor (CAR) directed against a tumor marker present on a target tumor cell. In several embodiments, the population comprises, consists of, or consists essentially of Natural Killer cells. In several embodiments, the population further comprises T cells. In several embodiments, the CAR is directed against CD19. In several embodiments, the CAR comprises one or more humanized CDR sequences. In several embodiments, the CAR is directed against an NKG2D ligand. In several embodiments, the genetic modification to the cells is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, and combinations thereof. In several embodiments, the Cas is Cas9. In several embodiments, the modification is to CISH and the CRISPR-Cas system is guided by one or more guide RNAs selected from those comprising a sequence of SEQ ID NO. 153, 154, 155, 156, or 157; the modification is to the TGFBR2 and the CRISPR-Cas system is guided by one or more guide RNAs selected from those comprising a sequence of SEQ ID NO. 147, 148, 149, 150, 151, or 152; the modification is to NKG2A and the CRISPR-Cas system is guided by one or more guide RNAs selected from those comprising a sequence of SEQ ID NO. 158, 159, or 160; the modification is to CBLB and the CRISPR-Cas system is guided by one or more guide RNAs selected from those comprising a sequence of SEQ ID NO. 164, 165, or 166; the modification is to TRIM29 and the CRISPR-Cas system is guided by one or more guide RNAs selected from those comprising a sequence of SEQ ID NO. 167, 168, or 169, and/or the modification is to SOCS2 and the CRISPR-Cas system is guided by one or more guide RNAs selected from those comprising a sequence of SEQ ID NO. 171, 172, or 173.

In several embodiments, the genetic modification(s) is made using a zinc finger nuclease (ZFN). In several embodiments, the genetic modification(s) is made using a Transcription activator-like effector nuclease (TALEN).

In several embodiments, the genetically altered immune cells exhibit increased cytotoxicity, increased viability and/or increased anti-tumor cytokine release profiles as compared to unmodified immune cells. In several embodiments, the genetically altered immune cells have been further genetically modified to reduce alloreactivity against the cells when administered to a subject that was not the donor of the cells.

Also provided for herein is a mixed population of immune cells for cancer immunotherapy, comprising a population of T cells that are substantially non-alloreactive through at least one modification to a subunit of a T Cell Receptor (TCR) selected from TCRα, TCRβ, TCRγ, and TORζ such that the TCR does not recognize major histocompatibility complex differences between the T cells of a recipient subject to which the mixed population of immune cells was administered, wherein the population of T cells is engineered to express a chimeric antigen receptor (CAR) directed against a tumor marker, wherein the tumor marker is selected from the group consisting of CD19, CD123, CD70, Her2, mesothelin, Claudin 6, BCMA, PD-L1, EGFR, and combinations thereof; and a population of natural killer (NK) cells, wherein the population of NK cells is engineered to express a chimeric receptor comprising an extracellular ligand binding domain, a transmembrane domain, a cytotoxic signaling complex and wherein the extracellular ligand binding domain a that is directed against a tumor marker selected from the group consisting of MICA, MICB, ULBP1, ULBP2, ULBP3, ULBP4, ULBP5, and ULBP6. In several embodiments, the TCR subunit modified is TCRα.

In several embodiments, the T cells and/or the NK cells are modified such that they express reduced levels of MHC I and/or MHC II molecules and thereby induce reduced immune response from a recipient subject's immune system to which the NK cells and T cells are allogeneic. In several embodiments, the MHC I and/or MHC II molecule is beta-microglobulin and/or CIITA (class II major histocompatibility complex transactivator). In several embodiments, the T cells and/or the NK cells further comprise a modification that disrupts expression of at least one immune checkpoint protein by the T cells and/or the NK cells. Depending on the embodiment the at least one immune checkpoint protein is selected from CTLA4, PD-1, lymphocyte activation gene (LAG-3), NKG2A receptor, KIR2DL-1, KIR2DL-2, KIR2DL-3, KIR2DS-1 and/or KIR2DA-2, and combinations thereof.

In several embodiments, the NK cells and/or T cells are further modified to reduce or substantially eliminate expression and/or function of CIS. In several embodiments, the NK cells are further engineered to express membrane bound IL-15.

In several embodiments, the CAR expressed by the T cells comprises (i) an tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain. In several embodiments, the T cells also express membrane bound IL15. In several embodiments, mbIL15 is encoded by the same polynucleotide encoding the CAR. In several embodiments, the anti-CD19 antibody comprises a variable heavy (VH) domain of a single chain Fragment variable (scFv) and a variable light (VL) domain of a scFv. In some such embodiments, the VH domain comprises, consists of, or consists essentially of the amino acid sequence of SEQ ID NO: 120. In several embodiments, the encoded VL domain comprises, consists of, or consists essentially of the amino acid sequence of SEQ ID NO: 118. In several embodiments, the OX40 subdomain is encoded by a sequence having at least 80%, 85%, 90%, or 95% sequence identity to SEQ ID NO. 5. In several embodiments, the CD3 zeta subdomain is encoded by a sequence having at least 80%, 85%, 90%, or 95% sequence identity to SEQ ID NO. 7. In several embodiments, mbIL15 is encoded by a sequence having at least 80%, 85%, 90%, or 95% sequence identity to SEQ ID NO. 11. In several embodiments, the CAR expressed by the T cells has at least 80%, 85%, 90%, or 95% sequence identity to the amino acid sequence set forth in SEQ ID NO: 178. In several embodiments, chimeric receptor expressed by the NK cells comprises (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the NK cells are further engineered to express membrane bound IL15 (which is optionally encoded by the same polynucleotide encoding the chimeric receptor). In several embodiments, the chimeric receptor is encoded by a polynucleotide having at least 80%, 85%, 90%, or 95% sequence identity to SEQ ID NO: 145. In several embodiments, the chimeric receptor has at least 80%, 85%, 90%, or 95% sequence identity to SEQ ID NO: 174.

In several embodiments, the modification to the TCR results in at least 80% of the population of T cells not expressing a detectable level of the TCR, but at least 70% of the population of T cells express a detectable level of the CAR. In several embodiments, the T cells and/or NK cells are further modified to reduce expression of one or more of a B2M surface protein, a cytokine-inducible SH2-containing protein (CIS) encoded by a CISH gene, a transforming growth factor beta receptor, a Natural Killer Group 2, member A (NKG2A) receptor, a Cbl proto-oncogene B protein encoded by a CBLB gene, a tripartite motif-containing protein 29 protein encoded by a TRIM29 gene, a suppressor of cytokine signaling 2 protein encoded by a SOCS2 gene by the T cells and/or NK cells. In several embodiments, gene editing can reduce expression of any of these target proteins by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. In several embodiments, target protein expression can be enhanced by about 30%, about 40%, about 50%, about 60%, about 70%, about 75%, about 80%, about 85%, about 90%, about 95%, about 97%, about 98%, about 99%, or more (including any amount between those listed). For example in several embodiments, the T cells and/or NK cells are further genetically edited to express CD47. In several embodiments, the NK cells are further genetically engineered to express HLA-E. Any genes that are knocked in can be knocked in in combination with any of the genes that are knocked out or otherwise disrupted.

In several embodiments, the modification(s) to the TCR, or the further modification of the NK cells or T cells is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, and combinations thereof. In several embodiments, the Cas is Cas9. In several embodiments, the CRISPR-Cas system comprises a Cas selected from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, the modification(s) to the TCR, or the further modification of the NK cells or T cells is made using a zinc finger nuclease (ZFN). In several embodiments, the modification(s) to the TCR, or the further modification of the NK cells or T cells is made using a Transcription activator-like effector nuclease (TALEN).

Also provided for herein is a mixed population of immune cells for cancer immunotherapy, comprising a population of T cells that are substantially non-alloreactive due to at least one modification to a subunit of a T Cell Receptor (TCR) such that the non-alloreactive T cells do not exhibit alloreactive effects against cells of a recipient subject, wherein the population of T cells is engineered to express a chimeric antigen receptor (CAR) directed against a tumor marker selected from CD19, CD123, CD70, Her2, mesothelin, Claudin 6, BCMA, PD-L1, EGFR, and combinations thereof, and a population of natural killer (NK) cells, wherein the population of NK cells is engineered to express a chimeric receptor comprising an extracellular ligand binding domain, a transmembrane domain, a cytotoxic signaling complex and wherein the extracellular ligand binding domain a that is directed against a tumor marker selected from the group consisting of MICA, MICB, ULBP1, ULBP2, ULBP3, ULBP4, ULBP5, and ULBP6.

Also provided herein are methods of treating cancer in a subject without inducing graft versus host disease, comprising administering to the subject the mixed population of immune cells according to the present disclosure. Provided for herein are uses of the mixed population of immune cells according to the present disclosure in the treatment of cancer. Provided for herein are uses of the mixed population of immune cells according to the present disclosure in the manufacture of a medicament for the treatment of cancer.

In several embodiments, there is provided a method for treating cancer in a subject comprising administering to the subject at least a first dose of a mixed population of immune cells, wherein the mixed population of cells comprises a population of substantially non-alloreactive T cells engineered to express a chimeric antigen receptor (CAR) directed against a tumor marker selected from CD19, CD123, CD70, Her2, mesothelin, Claudin 6, BCMA, PD-L1, EGFR, and combinations thereof and a population of natural killer (NK) cells engineered to express a chimeric receptor comprising an extracellular ligand binding domain, a transmembrane domain, a cytotoxic signaling complex and wherein the extracellular ligand binding domain a that is directed against a tumor marker selected from the group consisting of MICA, MICB, ULBP1, ULBP2, ULBP3, ULBP4, ULBP5, and ULBP6.

In several embodiments, the non-alloreactive T cells comprise at least one modification to a subunit of a T Cell Receptor (TCR) such that the non-alloreactive T cells do not exhibit alloreactive effects against cells of a recipient subject. In several embodiments, the CAR expressed by the T cells is directed against CD19. In several embodiments, the CAR expressed by the T cells comprises (i) an tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain. In several embodiments, the polynucleotide encoding the CAR also encodes membrane bound IL15. In several embodiments, the anti-CD19 antibody comprises a variable heavy (VH) domain of a single chain Fragment variable (scFv) and a variable light (VL) domain of a scFv. In several embodiments, the VH domain comprises, consists of, or consists essentially of the amino acid sequence of SEQ ID NO: 120 and wherein the VL domain comprises, consists of, or consists essentially of the amino acid sequence of SEQ ID NO: 118. In several embodiments, the CAR expressed by the T cells has at least 80%, 85%, 90%, or 95% sequence identity to the amino acid sequence set forth in SEQ ID NO: 178. In several embodiments, the chimeric receptor expressed by the NK cells comprises (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the polynucleotide encoding the chimeric receptor also encodes membrane bound IL15. In several embodiments, the chimeric receptor is encoded by a polynucleotide having at least 80%, 85%, 90%, or 95% sequence identity to SEQ ID NO: 145. In several embodiments, the chimeric receptor has at least 95%80%, 85%, 90%, or 95% sequence identity to SEQ ID NO: 174. In several embodiments, the OX40 subdomain of the CAR and/or chimeric receptor is encoded by a sequence having at least 80%, 85%, 90%, or 95% sequence identity to SEQ ID NO. 5. In several embodiments, the CD3 zeta subdomain of the CAR and/or chimeric receptor is encoded by a sequence having at least 80%, 85%, 90%, or 95% sequence identity to SEQ ID NO. 7. In several embodiments, the mbIL15 expressed by the T cells and/or the NK cells is encoded by a sequence having at least 80%, 85%, 90%, or 95% sequence identity to SEQ ID NO. 11.

In several embodiments, there is provided a mixed population of immune cells for cancer immunotherapy, wherein the mixed population comprises a population of T cells that express a CAR directed against a tumor antigen, the T cells having been genetically modified to be substantially non-alloreactive and a population of NK cells expressing a CAR directed against the same tumor antigen. In several embodiments, there is provided a mixed population of immune cells for cancer immunotherapy, wherein the mixed population comprises a population of T cells that express a CAR directed against a tumor antigen, the T cells having been genetically modified to be substantially non-alloreactive and a population of NK cells expressing a CAR directed against an additional tumor antigen. In several embodiments, there is provided a mixed population of immune cells for cancer immunotherapy, wherein the mixed population comprises a population of T cells that are substantially non-alloreactive and a population of NK cells expressing a chimeric receptor targeting a tumor ligand.

In several embodiments, the non-alloreactive T cells comprise at least one modification to a subunit of a T Cell Receptor (TCR) such that the TCR recognizes an antigen without recognition of major histocompatibility complex differences between the T cells of a subject to which the mixed population of immune cells was administered. In several embodiments, the population of non-alloreactive T cells is engineered to express a chimeric antigen receptor (CAR) directed against a tumor marker (e.g., a tumor associated antigen or a tumor antigen). Depending on the embodiment, the CAR can be engineered to target one or more of CD19, CD123, CD70, Her2, mesothelin, Claudin 6 (but not other Claudins), BCMA, PD-L1, EGFR.

In several embodiments, the population of NK cells is engineered to express a chimeric receptor comprising an extracellular ligand binding domain, a transmembrane domain, a cytotoxic signaling complex and wherein the extracellular ligand binding domain a that is directed against a tumor marker selected from the group consisting of MICA, MICB, ULBP1, ULBP2, ULBP3, ULBP4, ULBP5, and ULBP6. In several embodiments, the NK cells can also be engineered to express a CAR, the CAR can be engineered to target one or more of CD19, CD123, CD70, Her2, mesothelin, Claudin 6 (but not other Claudins), BCMA, PD-L1, EGFR (or any other antigen such that both T cells and NK cells are targeting the same antigen of interest).

In several embodiments, the T cells further comprise a mutation that disrupts expression of at least one immune checkpoint protein by the T cells. For example, the T cells may be mutated with respect to an immune checkpoint protein selected from CTLA4, PD-1 and combinations thereof. In several embodiments, blocking of B7-1/B7-2 to CTLA4 is also used to reduce T cells being maintained in an inactive state. Thus, in several embodiments, T cells are modified such that they express a mismatched or mutated CTLA4, while in some embodiments, an exogenous agent can be used to, for example, bind to and/or otherwise inhibit the ability of B7-1/B7-2 on antigen presenting cells to interact with CTLA4. Likewise, in several embodiments, NK cells can be modified to disrupt expression of at least one checkpoint inhibitor. In several embodiments, for example CDTLA4 or PD-1 are modified, e.g., mutated, in order to decrease the ability of such checkpoint inhibitors to reduce NK cell cytotoxic responses. In several embodiments, Lymphocyte activation gene 3 (LAG-3, CD223), is disrupted in NK cells (and/or T cells). In several embodiments, the inhibitory NKG2A receptor is mutated, knocked-out or inhibited, for example by an antibody. Monalizumab, by way of non-limiting example, is used in several embodiments to disrupt inhibitory signaling by the NKG2A receptor. In several embodiments, one or more of the killer inhibitory receptors (KIRs) on a NK cells is disrupted (e.g., through genetic modification) and/or blocked. For example, in several embodiments, one or more of KIR2DL-1, KIR2DL-2, KIR2DL-3, KIR2DS-1 and/or KIR2DA-2, are disrupted or blocked, thereby preventing their binding to HLA-C MHC I molecules. In addition, in several embodiments, TIM3 is modified, mutated (e.g., through gene editing) or otherwise functionally disrupted (e.g., blocked by an antibody) such that its normal function of suppressing the responses of immune cells upon ligand binding is disrupted. In several such embodiments, disruption of TIM3 expression or function (e.g., through CRISPr or other methods disclosed herein), optionally in combination with disruption of one or more immune checkpoint modulator, administered T cells and/or NK cells have enhanced anti-tumor activity. Tim-3 participates in galectin-9 secretion, the latter functioning to impair the anti-cancer activity of cytotoxic lymphoid cells including natural killer (NK) cells. TIM3 is also expressed in a soluble form, which prevents secretion of interleukin-2 (IL-2). Thus, in several embodiments, the disruption of TIM3, expression, secretion, or pathway functionality provides enhanced T cell and/or NK cell activity.

In several embodiments, TIGIT (also called VSTM3) is modified, mutated (e.g., through gene editing) or otherwise functionally disrupted (e.g., blocked by an antibody) such that its normal function of suppressing the responses of immune cells upon ligand binding is disrupted. CD155 is a ligand for TIGIT. In several embodiments, TIGIT expression is reduced or knocked out. In several embodiments, TIGIT is blocked by a non-activating ligand or its activity is reduced through a competitive inhibitor of CD155 (that inhibitor not activating TIGIT). TIGIT contains an inhibit ITIM motif, which in some embodiments is excised, for example, through gene editing with CRISPr, or other methods disclosed herein. In such embodiments, the function of TIGIT is reduced, which allows for enhanced T cell and/or NK cell activity.

In several embodiments, the adenosine receptor A1 is modified, mutated (e.g., through gene editing) or otherwise functionally disrupted (e.g., blocked by an antibody) such that its normal function of suppressing the responses of immune cells upon ligand binding is disrupted. Adenosine signaling is involved in tumor immunity, as a result of its function as an immunosuppressive metabolite. Thus, in several embodiments, the Adenosine Receptor A1 expression is reduced or knocked out. In several embodiments, the adenosine receptor A1 is blocked by a non-activating ligand or its activity is reduced through a competitive inhibitor of adenosine (that inhibitor not activating adenosine signaling pathways). In several embodiments, the adenosine receptor is modified, for example, through gene editing with CRISPr, or other methods disclosed herein to reduce its function or expression, which allows for enhanced T cell and/or NK cell activity.

In several embodiments, the TCR subunit modified is selected from TCRα, TCRβ, TCRγ, and TORδ. In several embodiments, the TCR subunit modified is TCRα.

In several embodiments, the modification to the TCR is made using a CRISPR-Cas system. In several embodiments, the disruption of expression of at least one immune checkpoint protein by the T cells or NK cells is made using a CRISPR-Cas system. For example, a Cas can be selected from Cas9, Csn2, Cas4, Cpf1, C2c1, C2c3, Cas13a, Cas13b, Cas13c, and combinations thereof. In several embodiments, the Cas is Cas9. In several embodiments, the CRISPR-Cas system comprises a Cas selected from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof.

In several embodiments, the modification to the TCR is made using a zinc finger nuclease (ZFN). In several embodiments, the disruption of expression of the at least one immune checkpoint protein by the T cells or NK cells is made using a zinc finger nuclease (ZFN). In several embodiments, the modification to the TCR is made using a Transcription activator-like effector nuclease (TALEN). In several embodiments, the disruption of expression of the at least one immune checkpoint protein by the T cells or NK cells is made using a Transcription activator-like effector nuclease (TALEN). Combinations of ZFNs and TALENs (and optionally CRISPR-Cas) are used in several embodiments to modify either or both NK cells and T cells.

According to several embodiments, either the NK cells, the non-alloreactive T cells, or both, are further engineered to express membrane bound IL-15.

Advantageously, the mixed cell populations are useful in the methods provided for herein, wherein cancer in a subject can be treated without inducing graft versus host disease. In several embodiments, the methods comprise administering to the subject mixed population of non-alloreactive T cells expressing a CAR and engineered NK cells expressing a chimeric receptor. Also provided for are uses of a mixed population of non-alloreactive T cells expressing a CAR and engineered NK cells expressing a chimeric receptor in the treatment of cancer and/or in the manufacture of a medicament for the treatment of cancer. In still additional embodiments, the NK cells and T cells are allogeneic with respect to the subject receiving them. In several embodiments, such combinations involved NK cells and T cells directed against the same target antigen. For example, in several embodiments both the NK cells and T cells (e.g., non-alloreactive T cells) are allogeneic with respect to the subject receiving them and are engineered to express a CAR that targets the same antigen—for example CD19. In some embodiments, the NK cells and T cells are configured to both target cells expressing another marker, such as CD123, CD70, Her2, mesothelin, Claudin 6 (but not other Claudins), BCMA, PD-L1, EGFR (or any other antigen such that both T cells and NK cells are targeting the same antigen of interest).

In several embodiments, the modification to the TCR results in at least 75%, at least 80%, at least 85%, at least 90%, or at least 95% of the population of T cells that do not express a detectable level of the TCR, while at the same time at least 55%, at least 60%, at least 65%, at least 70%, or at least 75% of the population of T cells express a detectable level of the CAR. These cells are thus primarily non-alloreactive and armed with an anti-tumor-directed CAR. Further aiding in limiting immune reactions from the allogeneic T cells, in several embodiments, wherein at least 50% of the engineered T cells express a detectable level of the CAR and do not express a detectable level of TCR surface protein or B2M surface protein.

In several embodiments, NK cells are genetically modified to reduce the immune response that an allogeneic host might develop against non-self NK cells. In several embodiments, the NK cells are engineered such that they exhibit reduced expression of one or more MCH Class I and/or one or more MHC Class II molecule. In several embodiments, the expression of beta-microglobulin is substantially, significantly or completely reduced in at least a portion of NK cells that express (or will be modified to express) a CAR directed against a tumor antigen, such as CD19 (or any other antigen disclosed herein). In several embodiments, the expression of CIITA (class II major histocompatibility complex transactivator) is substantially, significantly or completely reduced in at least a portion of NK cells that express (or will be modified to express) a CAR directed against a tumor antigen, such as CD19 (or any other antigen disclosed herein). In several embodiments, such genetically modified NK cells are generated using CRISPr-Cas systems, TALENs, zinc fingers, RNAi or other gene editing techniques. As discussed herein, in several embodiments, the NK cells with reduced allogenicity are used in combination with non-alloreactive T cells. In several embodiments, NK cells are modified to express CD47, which aids in the modified NK cell avoiding detection by endogenous innate immune cells of a recipient. In several embodiments, T cells are modified in a like fashion. In several embodiments, both NK cells and T cells are modified to express CD47, which aids in NK and/or T cell persistence in a recipient, thus enhancing anti-tumor effects. In several embodiments, NK cells are modified to express HLA-G, which aids in the modified NK cell avoiding detection by endogenous innate immune cells of a recipient. In several embodiments, T cells are modified in a like fashion. In several embodiments, both NK cells and T cells are modified to express HLA-G, which aids in NK and/or T cell persistence in a recipient, thus enhancing anti-tumor effects. In several embodiments, T cells and NK cells with reduced alloreactivty and engineered to express CARs against the same antigen are used to treat a cancer in an allogeneic patient.

In several embodiments, there is provided a population of genetically altered immune cells for cancer immunotherapy, comprising a population of immune cells that are genetically modified to reduce the expression of a transforming growth factor beta receptor by the immune cell, and genetically engineered to express a chimeric antigen receptor (CAR) directed against a tumor marker present on a target tumor cell. In additional embodiments, there is provided a population of genetically altered immune cells for cancer immunotherapy, comprising a population of immune cells that are genetically modified to reduce the expression of a Natural Killer Group 2, member A (NKG2A) receptor by the immune cell, and genetically engineered to express a chimeric antigen receptor (CAR) directed against a tumor marker present on a target tumor cell. In additional embodiments, there is provided a population of genetically altered immune cells for cancer immunotherapy, comprising a population of immune cells that are genetically modified to reduce the expression of a cytokine-inducible SH2-containing protein encoded by a CISH gene by the immune cell, and genetically engineered to express a chimeric antigen receptor (CAR) directed against a tumor marker present on a target tumor cell. CISH is an inhibitory checkpoint in NK cell-mediated cytotoxicity. In additional embodiments, there is provided a population of genetically altered immune cells for cancer immunotherapy, comprising a population of immune cells that are genetically modified to reduce the expression of a Cbl proto-oncogene B protein encoded by a CBLB gene by the immune cell, and genetically engineered to express a chimeric antigen receptor (CAR) directed against a tumor marker present on a target tumor cell. CBLB is an E3 ubiquitin ligase and a negative regulator of NK cell activation. In additional embodiments, there is provided a population of genetically altered immune cells for cancer immunotherapy, comprising a population of immune cells that are genetically modified to reduce the expression of a tripartite motif-containing protein 29 protein encoded by a TRIM29 gene by the immune cell, and genetically engineered to express a chimeric antigen receptor (CAR) directed against a tumor marker present on a target tumor cell. TRIM29 is an E3 ubiquitin ligase and a negative regulator of NK cell function after activation. In additional embodiments, there is provided a population of genetically altered immune cells for cancer immunotherapy, comprising a population of immune cells that are genetically modified to reduce the expression of a suppressor of cytokine signaling 2 protein encoded by a SOCS2 gene by the immune cell, and genetically engineered to express a chimeric antigen receptor (CAR) directed against a tumor marker present on a target tumor cell. SOCS2 is a negative regulator of NK cell function. In several embodiments the population of genetically altered immune cells comprises NK cells, T cells, or combinations thereof. In several embodiments, additional immune cell are also included, such as gamma delta T cells, NK T cells, and the like. In several embodiments, the CAR is directed against CD19. In some such embodiments, the CAR comprises one or more humanized CDR sequences. In additional embodiments, the CAR is directed against CD123. In several embodiments, the genetically modified cells are engineered to express more than one CAR that is directed to more than one target. Optionally, a mixed population of T cells and NK cells is used, in which the T cell and NK cells can each express at least one CAR, which may or may not be directed against the same cancer marker, depending on the embodiment. In several embodiments the cells express a CAR directed against an NKG2D ligand.

As discussed above, in several embodiments, the cells are edited using a CRISPr-based approach. In several embodiments, the modification is to TGFBR2 and the CRISPR-Cas system is guided by one or more guide RNAs selected from those comprising a sequence of SEQ ID NO. 147, 148, 149, 150, 151, or 152 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 147, 148, 149, 150, 151, or 152. In several embodiments, the modification is to NKG2A and the CRISPR-Cas system is guided by one or more guide RNAs selected from those comprising a sequence of SEQ ID NO. 158, 159, or 160 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 158, 159, or 160. In several embodiments, the modification is to CISH and the CRISPR-Cas system is guided by one or more guide RNAs selected from those comprising a sequence of SEQ ID NO. 153, 154, 155, 156, or 157 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 153, 154, 155, 156, or 157. In several embodiments, the modification is to CBLB and the CRISPR-Cas system is guided by one or more guide RNAs selected from those comprising a sequence of SEQ ID NO. 164, 165 or 166 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 164, 165, or 166. In several embodiments, the modification is to TRIM29 and the CRISPR-Cas system is guided by one or more guide RNAs selected from those comprising a sequence of SEQ ID NO. 167, 168, or 169 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 167, 168, or 169. In several embodiments, the modification is to SOCS2 and the CRISPR-Cas system is guided by one or more guide RNAs selected from those comprising a sequence of SEQ ID NO. 171, 172, or 173 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 171, 172, or 173. In some embodiments, the guide RNA is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides long.

In several embodiments, there is provided a method for producing an engineered T cell suitable for allogenic transplantation, the method comprising delivering to a T cell an RNA-guided nuclease, a gRNA targeting a T Cell Receptor gene, and a vector comprising a donor template that comprises a nucleic acid encoding a CAR, wherein the CAR comprises (i) a tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain, and (iv) membrane bound IL15, wherein the nucleic acid encoding the CAR is flanked by left and right homology arms to the T Cell Receptor gene locus; and (b) expanding the engineered T cells in culture.

Also provided is an additional method for an engineered T cell suitable for allogenic transplantation, the method comprising delivering to a T cell an RNA-guided nuclease, and a gRNA targeting a T Cell Receptor gene, in order to disrupt the expression of at least one subunit of the TCR, and delivering to the T cell a vector comprising a nucleic acid encoding a CAR, wherein the CAR comprises (i) a tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain, and (iv) membrane bound IL15 and expanding the engineered T cells in culture.

Further methods are also provided, for example a method for producing an engineered T cell suitable for allogenic transplantation, the method comprising delivering to a T cell a nuclease capable of inducing targeted double stranded DNA breaks at a target region of a T Cell Receptor gene, in order to disrupt the expression of at least one subunit of the TCR, delivering to the T cell a vector comprising a nucleic acid encoding a CAR, wherein the CAR comprises (i) a tumor binding domain that comprises an antibody fragment that recognizes one or more of CD19, CD123, CD70, Her2, mesothelin, Claudin 6, BCMA, PD-L1, and EGFR, (ii) a CD8 transmembrane domain, and (iii) a signaling complex that comprises an OX40 co-stimulatory subdomain and a CD3z co-stimulatory subdomain, and (iv) membrane bound IL15; and expanding the engineered T cells in culture. In several embodiments, the method further comprises modifying T-cells by inactivating at least a first gene encoding an immune checkpoint protein. In several embodiments, the immune checkpoint gene is selected from the group consisting of: PD1, CTLA-4, LAGS, Tim3, BTLA, BY55, TIGIT, B7H5, LAIR1, SIGLEC10, and 2B4.

Methods for treating cancers are provided, the methods comprising generating T cells suitable for allogeneic transplant according embodiments disclosed herein, wherein the T cells are from a donor, transducing a population of NK cells expanded from the same donor to express an activating chimeric receptor that comprises an extracellular ligand binding domain a that is directed against a tumor marker selected from the group consisting of MICA, MICB, ULBP1, ULBP2, ULBP3, ULBP4, ULBP5, and ULBP6 to generate an engineered NK cell population, optionally further expanding the T cells and/or the engineered NK cell population, combining the T cells suitable for allogeneic transplant with the engineered NK cell population, and administering the combined NK and T cell population to a subject allogeneic with respect to the donor.

Methods for treating cancers are provided, the methods comprising generating T cells suitable for allogeneic transplant according embodiments disclosed herein, wherein the T cells are from a donor and are modified to express a CAR directed against CD19, CD123, CD70, Her2, mesothelin, Claudin 6 (but not other Claudins), BCMA, PD-L1, or EGFR; transducing a population of NK cells expanded from the same donor to express a CAR directed against CD19, CD123, CD70, Her2, mesothelin, Claudin 6 (but not other Claudins), BCMA, PD-L1, or EGFR to generate an engineered NK cell population, optionally further expanding the T cells and/or the engineered NK cell population, combining the T cells suitable for allogeneic transplant with the engineered NK cell population, and administering the combined NK and T cell population to a subject allogeneic with respect to the donor.

There is also provided an additional method for treating a subject for cancer, the method comprising generating T cells suitable for allogeneic transplant according to embodiments disclosed herein, wherein the T cells are from a first donor, transducing a population of NK cells expanded from a second donor to express an activating chimeric receptor that comprises an extracellular ligand binding domain a that is directed against a tumor marker selected from the group consisting of MICA, MICB, ULBP1, ULBP2, ULBP3, ULBP4, ULBP5, and ULBP6 to generate an engineered NK cell population, optionally further expanding the T cells and/or the engineered NK cell population, combining the T cells suitable for allogeneic transplant with the engineered NK cell population, administering the combined NK and T cell population to a subject allogeneic with respect to the first and the second donor.

In several embodiments, there is provided herein an immune cell, and also populations of immune cells, that expresses a CD19-directed chimeric receptor, the chimeric receptor comprising an extracellular anti-CD19 binding moiety, a hinge and/or transmembrane domain, and an intracellular signaling domain. Also provided for herein are polynucleotides (as well as vectors for transfecting cells with the same) encoding a CD19-directed chimeric antigen receptor, the chimeric antigen receptor comprising an extracellular anti-CD19 binding moiety, a hinge and/or transmembrane domain, and an intracellular signaling domain.

Also provided for herein, in several embodiments, is a polynucleotide encoding a CD19-directed chimeric antigen receptor, the chimeric antigen receptor comprising an extracellular anti-CD19 binding moiety, wherein the anti-CD19 binding moiety comprises a scFv, a hinge, wherein the hinge is a CD8 alpha hinge, a transmembrane domain, and an intracellular signaling domain, wherein the intracellular signaling domain comprises a CD3 zeta ITAM.

Also provided for herein, in several embodiments, is a polynucleotide encoding a CD19-directed chimeric antigen receptor, the chimeric antigen receptor comprising an extracellular anti-CD19 binding moiety, wherein the anti-CD19 binding moiety comprises a variable heavy chain of a scFv or a variable light chain of a scFv, a hinge, wherein the hinge is a CD8 alpha hinge, a transmembrane domain, wherein the transmembrane domain comprises a CD8 alpha transmembrane domain, and an intracellular signaling domain, wherein the intracellular signaling domain comprises a CD3 zeta ITAM.

In several embodiments, the transmembrane domain comprises a CD8 alpha transmembrane domain. In several embodiments, the transmembrane domain comprises an NKG2D transmembrane domain. In several embodiments, the transmembrane domain comprises a CD28 transmembrane domain.

In several embodiments the intracellular signaling domain comprises or further comprises a CD28 signaling domain. In several embodiments, the intracellular signaling domain comprises or further comprises a 4-1 BB signaling domain. In several embodiments, the intracellular signaling domain comprises an or further comprises OX40 domain. In several embodiments, the intracellular signaling domain comprises or further comprises a 4-1BB signaling domain. In several embodiments, the intracellular signaling domain comprises or further comprises a domain selected from ICOS, CD70, CD161, CD40L, CD44, and combinations thereof.

In several embodiments, the polynucleotide also encodes a truncated epidermal growth factor receptor (EGFRt). In several embodiments, the EGFRt is expressed in a cell as a soluble factor. In several embodiments, the EGFRt is expressed in a membrane bound form. In several embodiments, the polynucleotide also encodes membrane-bound interleukin-15 (mbIL15). Also provided for herein are engineered immune cells (e.g., NK or T cells, or mixtures thereof) that express a CD19-directed chimeric antigen receptor encoded by a polynucleotide disclosed herein. Further provided are methods for treating cancer in a subject comprising administering to a subject having cancer engineered immune cells expressing the chimeric antigen receptors disclosed herein. In several embodiments, there is provided the use of the polynucleotides disclosed herein in the treatment of cancer and/or in the manufacture of a medicament for the treatment of cancer.

In several embodiments, the anti-CD19 binding moiety comprises a heavy chain variable (VH) domain and a light chain variable (VL) domain. In several embodiments, the VH domain has at least 95% identity to the VH domain amino acid sequence set forth in SEQ ID NO: 33. In several embodiments, the VL domain has at least 95% identity to the VL domain amino acid sequence set forth in SEQ ID NO: 32. In several embodiments, the anti-CD19 binding moiety is derived from the VH and/or VL sequences of SEQ ID NO: 33 or 32. For example, in several embodiments, the VH and VL sequences for SEQ ID NO: 33 and/or 32 are subject to a humanization campaign and therefore are expressed more readily and/or less immunogenic when administered to human subjects. In several embodiments, the anti-CD19 binding moiety comprises a scFv that targets CD19 wherein the scFv comprises a heavy chain variable region comprising the sequence of SEQ ID NO. 35 or a sequence at least 95% identical to SEQ ID NO: 35. In several embodiments, the anti-CD19 binding moiety comprises an scFv that targets CD19 comprises a light chain variable region comprising the sequence of SEQ ID NO. 36 or a sequence at least 95% identical to SEQ ID NO: 36. In several embodiments, the anti-CD19 binding moiety comprises a light chain CDR comprising a first, second and third complementarity determining region (LC CDR1, LC CDR2, and LC CDR3, respectively) and/or a heavy chain CDR comprising a first, second and third complementarity determining region (HC CDR1, HC CDR2, and HC CDR3, respectively). Depending on the embodiment, various combinations of the LC CDRs and HC CDRs are used. For example, in one embodiment the anti-CD19 binding moiety comprises LC CDR1, LC CDR3, HC CD2, and HC, CDR3. Other combinations are used in some embodiments. In several embodiments, the LC CDR1 comprises the sequence of SEQ ID NO. 37 or a sequence at least about 95% homologous to the sequence of SEQ NO. 37. In several embodiments, the LC CDR2 comprises the sequence of SEQ ID NO. 38 or a or a sequence at least about 95% homologous to the sequence of SEQ NO. 38. In several embodiments, the LC CDR3 comprises the sequence of SEQ ID NO. 39 or a sequence at least about 95% homologous to the sequence of SEQ NO. 39. In several embodiments, the HC CDR1 comprises the sequence of SEQ ID NO. 40 or a sequence at least about 95% homologous to the sequence of SEQ NO. 40. In several embodiments, the HC CDR2 comprises the sequence of SEQ ID NO. 41, 42, or 43 or a sequence at least about 95% homologous to the sequence of SEQ NO. 41, 42, or 43. In several embodiments, the HC CDR3 comprises the sequence of SEQ ID NO. 44 or a sequence at least about 95% homologous to the sequence of SEQ NO. 44.

In several embodiments, there is also provided an anti-CD19 binding moiety that comprises a light chain variable region (VL) and a heavy chain variable region (HL), the VL region comprising a first, second and third complementarity determining region (VL CDR1, VL CDR2, and VL CDR3, respectively and the VH region comprising a first, second and third complementarity determining region (VH CDR1, VH CDR2, and VH CDR3, respectively. In several embodiments, the VL region comprises the sequence of SEQ ID NO. 45, 46, 47, or 48 or a sequence at least about 95% homologous to the sequence of SEQ NO. 45, 46, 47, or 48. In several embodiments, the VH region comprises the sequence of SEQ ID NO. 49, 50, 51 or 52 or a sequence at least about 95% homologous to the sequence of SEQ NO. 49, 50, 51 or 52.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 53 or a sequence at least about 95% homologous to the sequence of SEQ NO. 53. In several embodiments, the LC CDR2 comprises the sequence of SEQ ID NO. 54 or a sequence at least about 95% homologous to the sequence of SEQ NO. 54. In several embodiments, the LC CDR3 comprises the sequence of SEQ ID NO. 55 or a sequence at least about 95% homologous to the sequence of SEQ NO. 55. In several embodiments, the HC CDR1 comprises the sequence of SEQ ID NO. 56 or a sequence at least about 95% homologous to the sequence of SEQ NO. 56. In several embodiments, the HC CDR2 comprises the sequence of SEQ ID NO. 57 or a sequence at least about 95% homologous to the sequence of SEQ NO. 57. In several embodiments, the HC CDR3 comprises the sequence of SEQ ID NO. 58 or a sequence at least about 95% homologous to the sequence of SEQ NO. 58.

In several embodiments, the intracellular signaling domain of the chimeric receptor comprises an OX40 subdomain. In several embodiments, the intracellular signaling domain further comprises a CD3zeta subdomain. In several embodiments, the OX40 subdomain comprises the amino acid sequence of SEQ ID NO: 6 (or a sequence at least about 95% homologous to the sequence of SEQ ID NO. 6) and the CD3zeta subdomain comprises the amino acid sequence of SEQ ID NO: 8 (or a sequence at least about 95% homologous to the sequence of SEQ ID NO: 8).

In several embodiments, the hinge domain comprises a CD8a hinge domain. In several embodiments, the CD8a hinge domain, comprises the amino acid sequence of SEQ ID NO: 2 or a sequence at least about 95% homologous to the sequence of SEQ ID NO: 2).

In several embodiments, the immune cell also expresses membrane-bound interleukin-15 (mbIL15). In several embodiments, the mbIL15 comprises the amino acid sequence of SEQ ID NO: 12 or a sequence at least about 95% homologous to the sequence of SEQ ID NO: 12.

In several embodiments, wherein the chimeric receptor further comprises an extracellular domain of an NKG2D receptor. In several embodiments, the immune cell expresses a second chimeric receptor comprising an extracellular domain of an NKG2D receptor, a transmembrane domain, a cytotoxic signaling complex and optionally, mbIL15. In several embodiments, the extracellular domain of the NKG2D receptor comprises a functional fragment of NKG2D comprising the amino acid sequence of SEQ ID NO: 26 or a sequence at least about 95% homologous to the sequence of SEQ ID NO: 26. In various embodiments, the immune cell engineered to express the chimeric antigen receptor and/or chimeric receptors disclosed herein is an NK cell. In some embodiments, T cells are used. In several embodiments, combinations of NK and T cells (and/or other immune cells) are used.

In several embodiments, there are provided herein methods of treating cancer in a subject comprising administering to the subject having an engineered immune cell targeting CD19 as disclosed herein. Also provided for herein is the use of an immune cell targeting CD19 as disclosed herein for the treatment of cancer. Likewise, there is provided for herein the use of an immune cell targeting CD19 as disclosed herein in the preparation of a medicament for the treatment of cancer. In several embodiments, the cancer treated is acute lymphocytic leukemia.

Some embodiments of the methods and compositions described herein relate to an immune cell. In some embodiments, the immune cell expresses a CD19-directed chimeric receptor comprising an extracellular anti-CD19 moiety, a hinge and/or transmembrane domain, and/or an intracellular signaling domain. In some embodiments, the immune cell is a natural killer (NK) cell. In some embodiments, the immune cell is a T cell.

In some embodiments, the hinge domain comprises a CD8a hinge domain. In some embodiments, the hinge domain comprises an Ig4 SH domain.

In some embodiments, the transmembrane domain comprises a CD8a transmembrane domain. In some embodiments, the transmembrane domain comprises a CD28 transmembrane domain. In some embodiments, the transmembrane domain comprises a CD3 transmembrane domain.

In some embodiments, the signaling domain comprises an OX40 signaling domain. In some embodiments, the signaling domain comprises a 4-1 BB signaling domain. In some embodiments, the signaling domain comprises a CD28 signaling domain. In some embodiments, the signaling domain comprises an NKp80 signaling domain. In some embodiments, the signaling domain comprises a CD16 IC signaling domain. In some embodiments, the signaling domain comprises a CD3zeta or CD3 ITAM signaling domain. In some embodiments, the signaling domain comprises an mbIL-15 signaling domain. In some embodiments, the signaling domain comprises a 2A cleavage domain. In some embodiments, the mIL-15 signaling domain is separated from the rest or another portion of the CD19-directed chimeric receptor by a 2A cleavage domain.

Some embodiments relate to a method comprising administering an immune cell as described herein to a subject in need. In some embodiments, the subject has cancer. In some embodiments, the administration treats, inhibits, or prevents progression of the cancer.

### BRIEF DESCRIPTION OF THE DRAWINGS

FIG. 1 depicts non-limiting examples of tumor-directed chimeric antigen receptors.

FIG. 2 depicts additional non-limiting examples of tumor-directed chimeric antigen receptors.

FIG. 3 depicts additional non-limiting examples of tumor-directed chimeric antigen receptors.

FIG. 4 depicts additional non-limiting examples of tumor-directed chimeric antigen receptors.

FIG. 5 depicts additional non-limiting examples of tumor-directed chimeric antigen receptors.

FIG. 6 depicts non-limiting examples of tumor-directed chimeric antigen receptors directed against non-limiting examples of tumor markers.

FIG. 7 depicts additional non-limiting examples of tumor-directed chimeric antigen receptors directed against non-limiting examples of tumor markers.

FIGS. 8A-8I schematically depict various pathways that are altered through the gene editing techniques disclosed herein. FIG. 8A shows a schematic of the inhibitory effects of TGF-beta release by tumor cells in the tumor microenvironment. FIG. 8B shows a schematic of the CIS/CISH negative regulatory pathways on IL-15 function. FIG. 8C depicts a non-limiting schematic process flow for generation of a engineered non-alloreactive T cells and engineered NK cells for use in a combination therapy according to several embodiments disclosed herein. FIG. 8D shows a schematic of the signaling pathways that can lead to graft vs. host disease. FIG. 8E shows a schematic of how several embodiments disclosed herein can reduce and/or eliminate graft vs. host disease. FIG. 8F shows a schematic of the signaling pathways that can lead to host vs. graft rejection. FIG. 8G shows a schematic of several embodiments disclosed herein that can reduce and/or eliminate host vs. graft rejection. FIG. 8H shows a schematic of how edited immune cells can act against other edited immune cells in mixed cell product. FIG. 8I shows a schematic of how several embodiments disclosed herein can reduce and/or eliminate host immune effects against edited immune cells.

FIGS. 9A-9G show flow cytometry data related to the use of various guide RNAs to reduce expression of TGFB2R by NK cells. FIG. 9A shows control data. FIG. 9B shows data resulting from use of guide RNA 1; FIG. 9C shows data resulting from use of guide RNA 2; FIG. 9D shows data resulting from use of guide RNA 3; FIG. 9E shows data resulting from use of guide RNA 1 and guide RNA 2; FIG. 9F shows data resulting from use of guide RNA 1 and guide RNA 3; and FIG. 9G shows data resulting from use of guide RNA 2 and guide RNA 3. Expression was evaluated 7 days after electroporation with the indicated guide RNAs.

FIGS. 10A-10G show next generation sequence data related to the reduction of expression of TGFB2R by NK cells in response to electroporation with various guide RNAs. FIG. 10A shows control data. FIG. 10B shows data resulting from use of guide RNA 1; FIG. 10C shows data resulting from use of guide RNA 2; FIG. 10D shows data resulting from use of guide RNA 3; FIG. 10E shows data resulting from use of guide RNA 1 and guide RNA 2; FIG. 10F shows data resulting from use of guide RNA 1 and guide RNA 3; and FIG. 10G shows data resulting from use of guide RNA 2 and guide RNA 3.

FIGS. 11A-11D show data comparing the cytotoxicity of NK cells against tumor cells in the presence or absence of TGFb after knockdown of TGFB2R expression by CRISPr/Cas9. FIG. 11A shows the change in cytotoxicity after TGFB2R knockdown using guide RNAs 1 and 2. FIG. 11B shows the change in cytotoxicity after TGFB2R knockdown using guide RNAs 1 and 3 FIG. 11C shows the change in cytotoxicity after TGFB2R knockdown using guide RNAs 2 and 3. FIG. 11D shows data for mock TGFBR2 knockdown.

FIGS. 12A-12F show flow cytometry data related to the reduced expression of TGFB2R by additional guide RNAs. FIG. 12A shows an unstained control of the same cells expressing TGFB2R. FIG. 12B shows positive control data for NK cells expressing TGFB2R in the absence of electroporation with the CRISPr/Cas9 gene editing elements. FIG. 12C shows knockdown of TGFB2R expression when guide RNA 4 was used. FIG. 12D shows knockdown of TGFB2R expression when guide RNA 5 was used. FIG. 12E shows knockdown of TGFB2R expression when guide RNA 6 was used. FIG. 12F shows knockdown of TGFB2R expression when a 1:1 ratio of guide RNA 2 and 3 was used. Data were collected at 4 days post electroporation with the CRISPr/Cas9 gene editing elements.

FIGS. 13A-13F show flow cytometry data related to the expression of a non-limiting example of a chimeric antigen receptor (here an anti-CD19 CAR, NK19-1) by NK cells when subject to CRISPr/Cas9-mediated knockdown of TGFB2R. FIG. 13A shows a negative control for NK cells not engineered to express NK19-1. FIG. 13B shows positive control data for NK cells engineered to express NK19-1, but not electroporated with the CRISPr/Cas9 gene editing elements. FIG. 13C shows data related to NK19-1 expression on NK cells subjected to electroporation with guide RNA 4 to knock down TGFB2R expression. FIG. 13D shows data related to NK19-1 expression on NK cells subjected to electroporation with guide RNA 5 to knock down TGFB2R expression. FIG. 13E shows data related to NK19-1 expression on NK cells subjected to electroporation with guide RNA 6 to knock down TGFB2R expression. FIG. 13F shows data related to NK19-1 expression on NK cells subjected to electroporation with guide RNAs 2 and 3 to knock down TGFB2R expression. Data were collected at 4 days post-transduction with the vector encoding NK19-1.

FIGS. 14A-14D show data related to the resistance of NK cells expressing a non-limiting example of a CAR (here an anti-CD19 CAR, NK19-1) to TGFb inhibition as a result of single guide RNA knockdown of TGFB2R expression. FIG. 14A shows cytotoxicity of the NK cells against Nalm6 tumor cells where the NK cells were cultured with the Nalm6 cells in TGFbeta in order to recapitulate the tumor microenvironment. FIGS. 14B and 14C show control data (14C) where the TGFB2 receptor was not knocked out and FIG. 14C shows selected data curves extracted from 14A in order to show the selected curves more clearly. FIG. 14D shows a schematic of the treatment of the NK cells. NK cells were subject to electroporation with CRISPr/Cas9 and a single guide RNA at Day 0 and were cultured in high IL-2 media for 1 day, followed by low-IL-2 culture with feeder cells (e.g., modified K562 cells expressing, for example, 4-1BBL and/or mbIL15). At Day 7, NK cells were transduced with a virus encoding the NK19-1 CAR construct. At Day 14, the cytotoxicity of the resultant NK cells was evaluated.

FIGS. 15A-15D show data related to the enhanced cytokine secretion by primary and NK19-1-expressing NK cells. FIG. 15A shows data related to secretion of IFNgamma. FIG. 15B shows data related to secretion of GM-CSF. FIG. 15C shows data related to secretion of Granzyme B. FIG. 15D shows data related to secretion of TNF-alpha.

FIGS. 16A-16D show data related to knockout of NKG2A expression by NK cells through use of CRISPr/Cas9. FIG. 16A shows expression of NKG2A by NK cells subjected to a mock gene editing protocol. FIG. 16B shows NKG2A expression by NK cells after editing with CRISPr/Cas9 and guide RNA 1. FIG. 16C shows NKG2A expression by NK cells after editing with CRISPr/Cas9 and guide RNA 2. FIG. 16D shows NKG2A expression by NK cells after editing with CRISPr/Cas9 and guide RNA 3.

FIGS. 17A-17B show data related to the cytotoxicity of NK cells with knocked-out NKG2A expression (as compared to mock cells). FIG. 17A shows cytotoxicity of the NKG2A-edited NK cells against REH cells at 7 days post-electroporation with the CRISPr/Cas9 gene editing elements. FIG. 17B shows flow cytometry data related to the degree of HLA-E expression on REH cells.

FIG. 18 shows data related to the cytotoxicity of mock NK cells or NK cells where Cytokine-inducible SH2-containing protein (CIS) expression was knocked out by gene editing of the CISH gene, which encodes CIS in humans. CIS is an inhibitory checkpoint in NK cell-mediated cytotoxicity. NK-cell cytotoxicity against REH tumor cells was measured at 7 days post-electroporation with the CRISPr/Cas9 gene editing elements.

FIGS. 19A-19E show data related to the impact of CISH-knockout on expression of a non-limiting example of a chimeric antigen receptor construct (here an anti-CD19 CAR, NK19-1) by NK cells. FIG. 19A shows CD19 CAR expression (as measured by FLAG expression, which is included in this construct for detection purposes, while additional embodiments of the CAR do not comprise a tag) in control (untransduced) NK cells. FIG. 19B shows anti-CD19 CAR expression in NK cells subjected to CISH knockdown using CRISPr/Cas9 and guide RNA 1. FIG. 19C shows anti-CD19 CAR expression in NK cells subjected to CISH knockdown using CRISPr/Cas9 and guide RNA 2. FIG. 19D shows anti-CD19 CAR expression in NK cells subjected to mock gene-editing conditions (electroporation only). FIG. 19E shows a Western Blot depicting the loss of the CIS protein band at 35 kDa, indicating knockout of the CISH gene.

FIGS. 20A-20B show data from a cytotoxicity assay using donor NK cells modified through gene editing and/or engineered to express a CAR against Nalm6 tumor cells. FIG. 20A shows data from a single challenge assay at a 1:2 effector:target ratio with data collected 7 days post-transduction of the indicated CAR constructs. FIG. 20B shows data from a double challenge model, where the control, edited, and/or edited/engineered NK cells were challenged with Nalm6 tumor cells at two time points.

FIGS. 21A-21B show data related CISH knockout NK cell survival and cytotoxicity over extended time in culture. FIG. 21A shows NK cell survival data over time when NK cells were treated as indicated. FIG. 21B shows NK cell cytotoxicity data against tumor cells after being cultured for 100 days.

FIGS. 22A-22E show cytokine release data by NK cells treated with the indicated control, gene editing, or gene editing+engineered to express a CAR conditions. FIG. 22A shows data related to interferon gamma release. FIG. 22B shows data related to tumor necrosis factor alpha release. FIG. 22C shows data related to GM-CSF release. FIG. 22D shows data related to Granzyme B release. FIG. 22E shows data related to perforin release.

FIGS. 23A-23C show data from a cytotoxicity assay of mock NK cells or NK cells where either Cbl proto-oncogene B (CBLB) or tripartite motif-containing protein 29 (TRIM29) expression was knocked out by CRISPR/Cas9 gene editing. FIG. 23A shows cytotoxicity data for NK cells knocked out with three different CBLB gRNAs, CISH gRNA 5, or mock NK cells. FIG. 23B shows cytotoxicity data for NK cells knocked out with three different TRIM19 gRNAs, CISH gRNA 5, or mock NK cells. FIG. 23C shows the timeline for electroporation and cytotoxicity assay.

FIGS. 24A-24C show data from a time course cytotoxicity assay of mock NK cells or NK cells where either suppressor of cytokine signaling 2 (SOCS2) or CISH expression was knocked out by CRISPR/Cas9 gene editing. FIG. 24A shows time course cytotoxicity data for NK cells knocked out with three different SOCS2 gRNAs, CISH gRNA 2, or CD45 gRNA using the MaxCyte electroporation system. FIG. 24B shows time course cytotoxicity data for NK cells knocked out with three different SOCs2 gRNAs, CISH gRNA 2 or CD45 gRNA using the Lonza electroporation system. FIG. 24C shows the timeline for electroporation and cytotoxicity assay.

### DETAILED DESCRIPTION

Some embodiments of the methods and compositions provided herein relate to engineered immune cells and combinations of the same for use in immunotherapy. In several embodiments, the engineered cells are engineered in multiple ways, for example, to express a cytotoxicity-inducing receptor complex. As used herein, the term “cytotoxic receptor complexes” shall be given its ordinary meaning and shall also refer to (unless otherwise indicated), Chimeric Antigen Receptors (CAR), chimeric receptors (also called activating chimeric receptors in the case of NKG2D chimeric receptors). In several embodiments, the cells are further engineered to achieve a modification of the reactivity of the cells against non-tumor tissue. Several embodiments relate to the modification of T cells, through various genetic engineering methodologies, such that the resultant T cells have reduced and/or eliminated alloreactivity. Such non-alloreactive T cells can also be engineered to express a chimeric antigen receptor (CAR) that enables the non-alloreactive T cells to impart cytotoxic effects against tumor cells. In several embodiments, natural killer (NK) cells are also engineered to express a city-inducing receptor complex (e.g., a chimeric antigen receptor or chimeric receptor). In several embodiments, combinations of these engineered immune cell types are used in immunotherapy, which results in both a rapid (NK-cell based) and persistent (T-cell based) anti-tumor effect, all while advantageously having little to no graft versus host disease. Some embodiments include methods of use of the compositions or cells in immunotherapy.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, and/or amelioration of various physiological symptoms associated with the cancerous condition.

### Cell Types

Some embodiments of the methods and compositions provided herein relate to a cell such as an immune cell. For example, an immune cell, such as a T cell, may be engineered to include a chimeric receptor such as a CD19-directed chimeric receptor, or engineered to include a nucleic acid encoding said chimeric receptor as described herein. Additional embodiments relate to engineering a second set of cells to express another cytotoxic receptor complex, such as an NKG2D chimeric receptor complex as disclosed herein. Still additional embodiments relate to the further genetic manipulation of T cells (e.g., donor T cells) to reduce, disrupt, minimize and/or eliminate the ability of the donor T cell to be alloreactive against recipient cells (graft versus host disease).

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells, Natural Killer (NK cells), or combinations thereof, as described in more detail below.

To facilitate cancer immunotherapies, there are provided for herein polynucleotides, polypeptides, and vectors that encode chimeric antigen receptors (CAR) that comprise a target binding moiety (e.g., an extracellular binder of a ligand, or a tumor marker-directed chimeric receptor, expressed by a cancer cell) and a cytotoxic signaling complex. For example, some embodiments include a polynucleotide, polypeptide, or vector that encodes, for example a chimeric antigen receptor directed against a tumor marker, for example, CD19, CD123, CD70, Her2, mesothelin, Claudin 6, BCMA, EGFR, among others, to facilitate targeting of an immune cell to a cancer and exerting cytotoxic effects on the cancer cell. Also provided are engineered immune cells (e.g., T cells or NK cells) expressing such CARs. There are also provided herein, in several embodiments, polynucleotides, polypeptides, and vectors that encode a construct comprising an extracellular domain comprising two or more subdomains, e.g., first CD19-targeting subdomain comprising a CD19 binding moiety as disclosed herein and a second subdomain comprising a C-type lectin-like receptor and a cytotoxic signaling complex. Also provided are engineered immune cells (e.g., T cells or NK cells) expressing such bi-specific constructs. Methods of treating cancer and other uses of such cells for cancer immunotherapy are also provided for herein.

To facilitate cancer immunotherapies, there are also provided for herein polynucleotides, polypeptides, and vectors that encode chimeric receptors that comprise a target binding moiety (e.g., an extracellular binder of a ligand expressed by a cancer cell) and a cytotoxic signaling complex. For example, some embodiments include a polynucleotide, polypeptide, or vector that encodes, for example an activating chimeric receptor comprising an NKG2D extracellular domain that is directed against a tumor marker, for example, MICA, MICB, ULBP1, ULBP2, ULBP3, ULBP4, ULBP5, and ULBP6, among others, to facilitate targeting of an immune cell to a cancer and exerting cytotoxic effects on the cancer cell. Also provided are engineered immune cells (e.g., T cells or NK cells) expressing such chimeric receptors. There are also provided herein, in several embodiments, polynucleotides, polypeptides, and vectors that encode a construct comprising an extracellular domain comprising two or more subdomains, e.g., first and second ligand binding receptor and a cytotoxic signaling complex. Also provided are engineered immune cells (e.g., T cells or NK cells) expressing such bi-specific constructs (in some embodiments the first and second ligand binding domain target the same ligand). Methods of treating cancer and other uses of such cells for cancer immunotherapy are also provided for herein.

Engineered Cells for Immunotherapy

In several embodiments, cells of the immune system are engineered to have enhanced cytotoxic effects against target cells, such as tumor cells. For example, a cell of the immune system may be engineered to include a tumor-directed chimeric receptor and/or a tumor-directed CAR as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric receptor, such as an NKG2D chimeric receptor, and/or a CAR, such as a CD19-directed CAR, or a nucleic acid encoding the chimeric receptor or the CAR. In several embodiments, the cells are optionally engineered to co-express a membrane-bound interleukin 15 (mbIL15) co-stimulatory domain. As discussed in more detail below, in several embodiments, the cells, particularly T cells, are further genetically modified to reduce and/or eliminate the alloreactivity of the cells.

Monocytes for Immunotherapy

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake cellular material, or entire cells, followed by digestion and destruction of the engulfed cellular material. In several embodiments, monocytes are used in connection with one or more additional engineered cells as disclosed herein. Some embodiments of the methods and compositions described herein relate to a monocyte that includes a tumor-directed CAR, or a nucleic acid encoding the tumor-directed CAR. Several embodiments of the methods and compositions disclosed herein relate to monocytes engineered to express a CAR that targets a tumor marker, for example, CD19, CD123, CD70, Her2, mesothelin, Claudin 6, BCMA, EGFR, among others, and a membrane-bound interleukin 15 (mbIL15) co-stimulatory domain. Several embodiments of the methods and compositions disclosed herein relate to monocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

Lymphocytes for Immunotherapy

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Several embodiments of the methods and compositions disclosed herein relate to lymphocytes engineered to express a CAR that targets a tumor marker, for example, CD19, CD123, CD70, Her2, mesothelin, Claudin 6, BCMA, EGFR, among others, and a membrane-bound interleukin 15 (mbIL15) co-stimulatory domain. Several embodiments of the methods and compositions disclosed herein relate to lymphocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

T Cells for Immunotherapy

T cells are distinguishable from other lymphocytes sub-types (e.g., B cells or NK cells) based on the presence of a T-cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Several embodiments of the methods and compositions disclosed herein relate to T cells engineered to express a CAR that targets a tumor marker, for example, CD19, CD123, CD70, Her2, mesothelin, Claudin 6, BCMA, EGFR, among others, and a membrane-bound interleukin 15 (mbIL15) co-stimulatory domain. Several embodiments of the methods and compositions disclosed herein relate to T cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

NK Cells for Immunotherapy

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells). Some embodiments of the methods and compositions described herein relate to NK cells engineered to express a CAR that targets a tumor marker, for example, CD19, CD123, CD70, Her2, mesothelin, Claudin 6, BCMA, EGFR, among others, and optionally a membrane-bound interleukin 15 (mbIL15) co-stimulatory domain. Several embodiments of the methods and compositions disclosed herein relate to NK cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a hematopoietic stem cell engineered to express a CAR that targets a tumor marker, for example, CD19, CD123, CD70, Her2, mesothelin, Claudin 6, BCMA, EGFR, among others, and optionally a membrane-bound interleukin 15 (mbIL15) co-stimulatory domain. Several embodiments of the methods and compositions disclosed herein relate to hematopoietic stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

Genetic Engineering of Immune Cells

As discussed above, a variety of cell types can be utilized in cellular immunotherapy. Further, as elaborated on in more detail below, and shown in the Examples, genetic modifications can be made to these cells in order to enhance one or more aspects of their efficacy (e.g., cytotoxicity) and/or persistence (e.g., active life span). As discussed herein, in several embodiments NK cells are used for immunotherapy. In several embodiments provided for herein, gene editing of the NK cell can advantageously impart to the edited NK cell the ability to resist and/or overcome various inhibitory signals that are generated in the tumor microenvironment. It is known that tumors generate a variety of signaling molecules that are intended to reduce the anti-tumor effects of immune cells. As discussed in more detail below, in several embodiments, gene editing of the NK cell limits this tumor microenvironment suppressive effect on the NK cells, T cells, combinations of NK and T cells, or any edited/engineered immune cell provided for herein. As discussed below, in several embodiments, gene editing is employed to reduce or knockout expression of target proteins, for example by disrupting the underlying gene encoding the protein. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed).

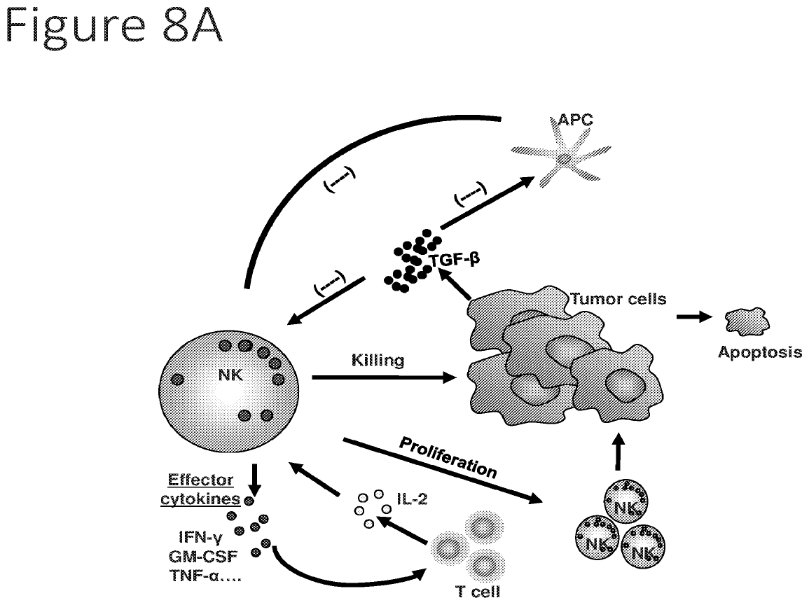

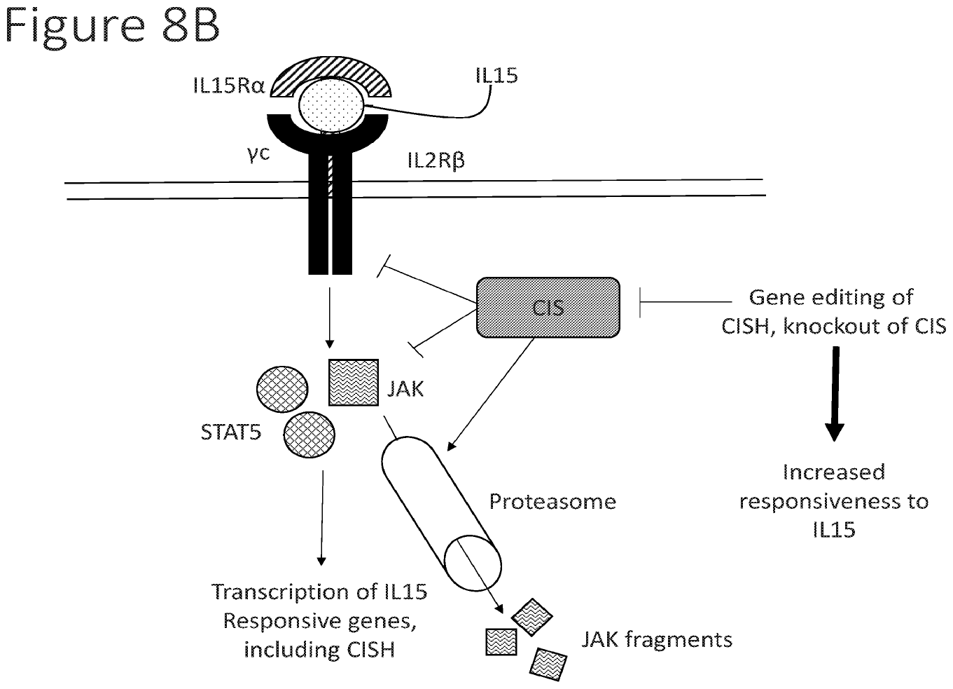

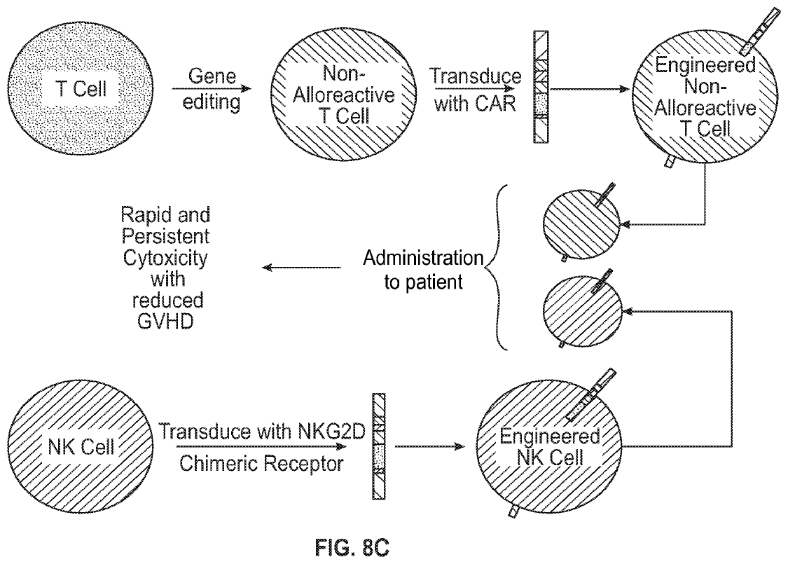

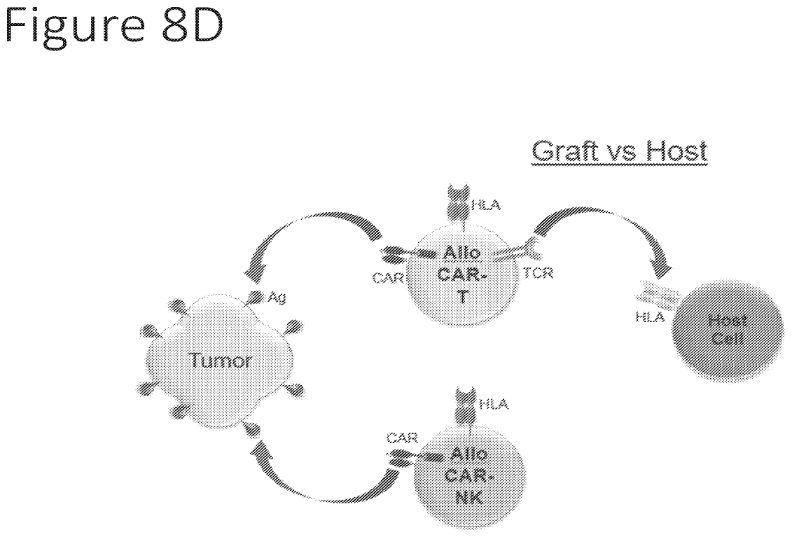

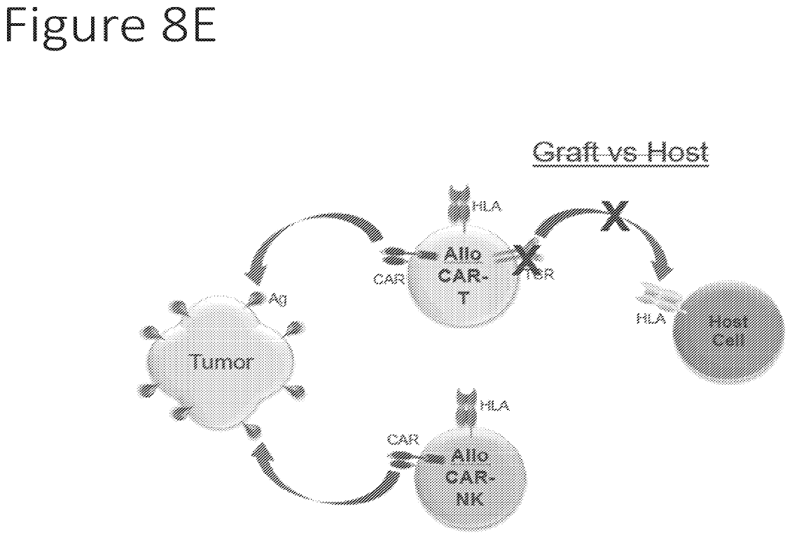

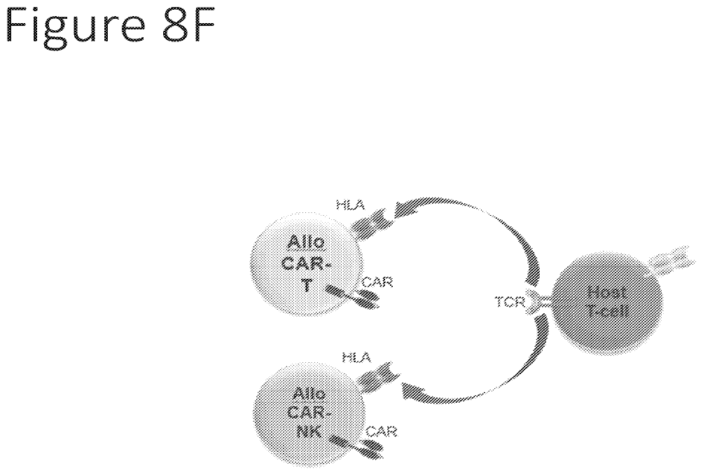

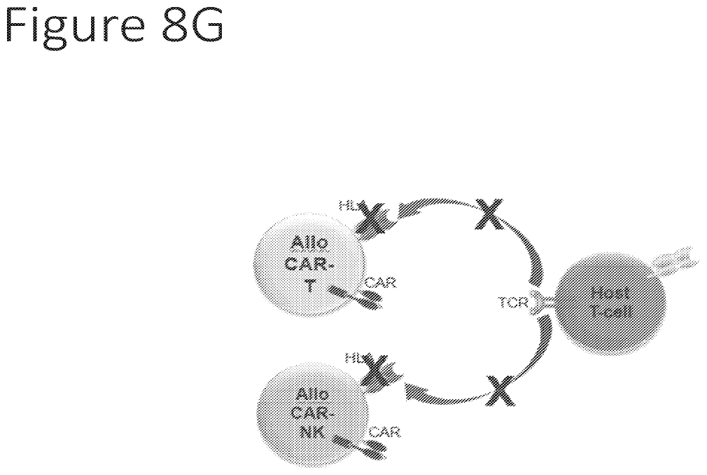

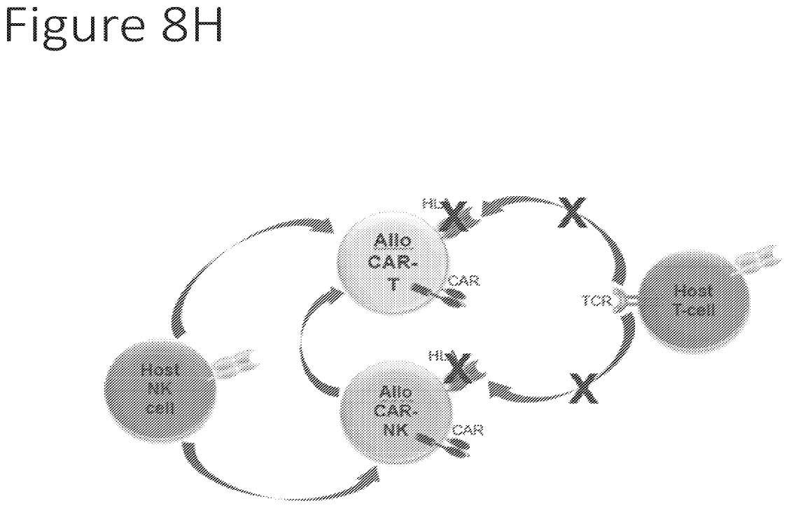

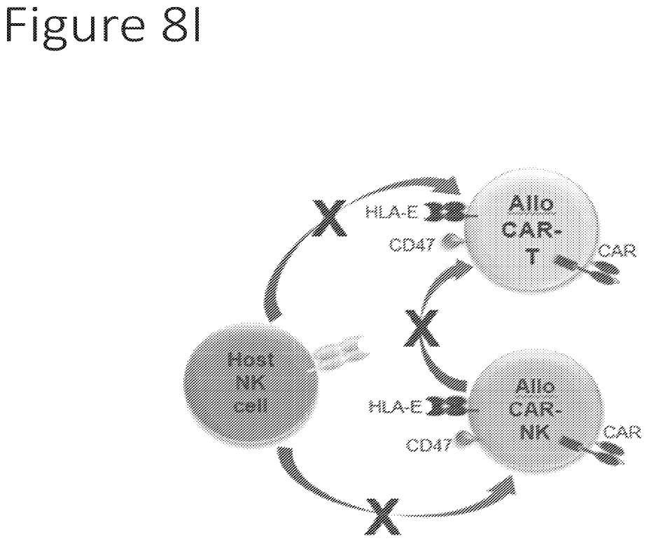

By way of non-limiting example, TGF-beta is one such cytokine released by tumor cells that results in immune suppression within the tumor microenvironment. That immune suppression reduces the ability of immune cells, even engineered CAR-immune cells is some cases, to destroy the tumor cells, thus allowing for tumor progression. In several embodiments, as discussed in detail below, immune checkpoint inhibitors are disrupted through gene editing. In several embodiments, blockers of immune suppressing cytokines in the tumor microenvironment are used, including blockers of their release or competitive inhibitors that reduce the ability of the signaling molecule to bind and inhibit an immune cell. Such signaling molecules include, but are not limited to TGF-beta, IL10, arginase, inducible NOS, reactive-NOS, Arg1, Indoleamine 2,3-dioxygenase (IDO), and PGE₂. However, in additional embodiments, there are provided immune cells, such as NK cells, wherein the ability of the NK cell (or other cell) to respond to a given immunosuppressive signaling molecule is disrupted and/or eliminated. For example, in several embodiments, in several embodiments, NK cells or T cells are genetically edits to become have reduced sensitivity to TGF-beta. TGF-beta is an inhibitor of NK cell function on at least the levels of proliferation and cytotoxicity. See, for example, FIG. 8A which schematically shows some of the inhibitory pathways by which TGF-beta reduces NK cell activity and/or proliferation. Thus, according to some embodiments, the expression of the TGF-beta receptor is knocked down or knocked out through gene editing, such that the edited NK is resistant to the immunosuppressive effects of TGF-beta in the tumor microenvironment. In several embodiments, the TGFB2 receptor is knocked down or knocked out through gene editing, for example, by use of CRISPR-Cas editing. Small interfering RNA, antisense RNA, TALENs or zinc fingers are used in other embodiments. Other isoforms of the TGF-beta receptor (e.g., TGF-beta 1 and/or TGF-beta 3) are edited in some embodiments. In some embodiments TGF-beta receptors in T cells are knocked down through gene editing.

In accordance with additional embodiments, other modulators of one or more aspects of NK cell (or T cell) function are modulated through gene editing. A variety of cytokines impart either negative (as with TGF-beta above) or positive signals to immune cells. By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. To keep NK cells in check under normal physiological circumstances, a cytokine-inducible SH2-containing protein (CIS, encoded by the CISH gene) acts as a critical negative regulator of IL-15 signaling in NK cells. As discussed herein, because IL15 biology impacts multiple aspects of NK cell functionality, including, but not limited to, proliferation/expansion, activation, cytotoxicity, persistence, homing, migration, among others. Thus, according to several embodiments, editing CISH enhances the functionality of NK cells across multiple functionalities, leading to a more effective and long-lasting NK cell therapeutic. In several embodiments, inhibitors of CIS are used in conjunction with engineered NK cell administration. In several embodiments, the CIS expression is knocked down or knocked out through gene editing of the CISH gene, for example, by use of CRISPR-Cas editing. Small interfering RNA, antisense RNA, TALENs or zinc fingers are used in other embodiments. In some embodiments CIS expression in T cells is knocked down through gene editing.

In several embodiments, CISH gene editing endows an NK cell with enhanced ability to home to a target site. In several embodiments, CISH gene editing endows an NK cell with enhanced ability to migrate, e.g., within a tissue in response to, for example chemoattractants or away from repellants. In several embodiments, CISH gene editing endows an NK cell with enhanced ability to be activated, and thus exert, for example, anti-tumor effects. In several embodiments, CISH gene editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH gene editing endows an NK cell with enhanced cytotoxicity. In several embodiments, the editing of CISH synergistically enhances the cytotoxic effects of engineered NK cells and/or engineered T cells that express a CAR.

In several embodiments, CISH gene editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, knockdown of CISH disinhibits JAK-STAT (e.g., JAK1-STAT5) signaling and there is enhanced transcription of IL15-responsive genes. In several embodiments, knockout of CISH yields enhanced signaling through mammalian target of rapamycin (mTOR), with corresponding increases in expression of genes related to cell metabolism and respiration. In several embodiments, knockout of CISH yields IL15 induced increased expression of IL-2Rα (CD25), but not IL-15Rα or IL-2/15Rβ, enhanced NK cell membrane binding of IL15 and/or IL2, increased phosphorylation of STAT-3 and/or STAT-5, and elevated expression of the antiapoptotic proteins, such as Bcl-2. In several embodiments, CISH knockout results in IL15-induced upregulation of selected genes related to mitochondrial functions (e.g., electron transport chain and cellular respiration) and cell cycle. Thus, in several embodiments, knockout of CISH by gene editing enhances the NK cell cytotoxicity and/or persistence, at least in part via metabolic reprogramming. In several embodiments, negative regulators of cellular metabolism, such as TXNIP, are downregulated in response to CISH knockout. In several embodiments, promotors for cell survival and proliferation including BIRC5 (Survivin), TOP2A, CKS2, and RACGAP1 are upregulated after CISH knockout, whereas antiproliferative or proapoptotic proteins such as TGFB1, ATM, and PTCH1 are downregulated. In several embodiments, CISH knockout alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT5, STAT1, TBX21, LCK, JAK3, IL& receptor, ABL1, IL9, STAT5A, STAT5B, Tcf7, PRDM1, and/or EOMES.

In several embodiments, gene editing of the immune cells can also provide unexpected enhancement in the expansion, persistence and/or cytotoxicity of the edited immune cell. As disclosed herein, engineered cells (e.g., those expressing a CAR) may also be edited, the combination of which provides for a robust cell for immunotherapy. In several embodiments, the edits allow for unexpectedly improved NK cell expansion, persistence and/or cytotoxicity. In several embodiments, knockout of CISH expression in NK cells removes a potent negative regulator of IL15-mediated signaling in NK cells, disinhibits the NK cells and allows for one or more of enhanced NK cell homing, NK cell migration, activation of NK cells, expansion, cytotoxicity and/or persistence. Additionally, in several embodiments, the editing can enhance NK and/or T cell function in the otherwise suppressive tumor microenvironment. In several embodiments, CISH gene editing results in enhanced NK cell expansion, persistence and/or cytotoxicity without requiring Notch ligand being provided exogenously.

As discussed above, T cells that are engineered to express a CAR or chimeric receptor are employed in several embodiments. Also as mentioned above, T cells express a T Cell Receptor (TCR) on their surface. As disclosed herein, in several embodiments, autologous immune cells are transferred back into the original donor of the cells. In such embodiments, immune cells, such as NK cells or T cells are obtained from patients, expanded, genetically modified (e.g., with a CAR or chimeric receptor) and/or optionally further expanded and re-introduced into the patient. As disclosed herein, in several embodiments, allogeneic immune cells are transferred into a subject that is not the original donor of the cells. In such embodiments, immune cells, such as NK cells or T cells are obtained from a donor, expanded, genetically modified (e.g., with a CAR or chimeric receptor) and/or optionally further expanded and administered to the subject.

Allogeneic immunotherapy presents several hurdles to be overcome. In immune-competent hosts, the administered allogeneic cells are rapidly rejected, known as host versus graft rejection (HvG). This substantially limits the efficacy of the administered cells, particularly their persistence. In immune-incompetent hosts, allogeneic cells are able to engraft. However, if the administered cells comprise a T cell (several embodiments disclosed herein employ mixed populations of NK and T cells), the endogenous T cell receptor (TCR) specificities recognize the host tissue as foreign, resulting in graft versus host disease (GvHD). GvHD can lead to significant tissue damage in the host (cell recipient). Several embodiments disclosed herein address both of these hurdles, thereby allowing for effective and safe allogeneic immunotherapy. In several embodiments, gene edits can advantageously help to reduce and/or avoid graft vs. host disease (GvHD). A non-limiting embodiment of such an approach, using a mixed population of NK cell and T cells, is schematically illustrated in FIG. 8C, wherein the NK cells are engineered to express a CAR and the T cells are engineered to not only express a CAR, but also edited to render the T cells non-alloreactive. FIG. 8D schematically shows a mechanism by which graft v. host disease occurs. An allogeneic T cell and an allogeneic NK cell, both engineered to express a CAR that targets the tumor, are introduced into a host. However, the T cell still bears the native T-cell receptor (TCR). This TCR recognizes the HLA type of the host cell as “non-self” and can exert cytotoxicity against host cells. FIG. 8E shows a non-limiting embodiment of how graft v. host disease can be reduced or otherwise avoided through gene editing of the T cells. Briefly, as this approach is discussed in more detail below, gene editing can be performed in order to knockout the native TCR on T cells. Lacking a TCR, the allogeneic T cell cannot detect the “non-self” HLA of the host cells, and therefore is not triggered to exert cytotoxicity against host cells. Thus, in several embodiments T cells are subjected to gene editing to either reduce functionality of and/or reduce or eliminate expression of the native T cell. In several embodiments, CRISPR is used to knockout the TCR. These, and other, embodiments are discussed below.

T cell receptors (TCR) are cell surface receptors that participate in the activation of T cells in response to the presentation of an antigen. The TCR is made up of two different protein chains (it is a heterodimer). The majority of human T cells have TCRs that are made up of an alpha (α) chain and a beta (β) chain (encoded by separate genes). A small percentage of T cells have TCRs made up of gamma and delta (γ/δ) chains (the cells being known as gamma-delta T cells).

Rather than recognizing an intact antigen (as with immunoglobulins), T cells are activated by processed peptide fragments in association with an MHC molecule. This is known as MHC restriction. When the TCR recognizes disparities between the donor and recipient MHC, that recognition stimulates T cell proliferation and the potential development of GVHD. In some embodiments, the genes encoding either the TCRα, TCRβ, TCRγ, and/or the TCEδ are disrupted or otherwise modified to reduce the tendency of donor T cells to recognize disparities between donor and host MHC, thereby reducing recognition of alloantigen and GVHD.

T-cell mediated immunity involves a balance between co-stimulatory and inhibitory signals that serve to fine-tune the immune response. Inhibitory signals, also known as immune checkpoints, allow for avoidance of auto-immunity (e.g., self-tolerance) and also limit immune-mediated damage. Immune checkpoint protein expression are often altered by tumors, enhancing immune resistance in tumor cells and limiting immunotherapy efficacy. CTLA4 downregulates the amplitude of T cell activation. In contrast, PD1 limits T cell effector functions in peripheral tissue during an inflammatory response and also limits autoimmunity. Immune checkpoint blockade, in several embodiments, helps to overcome a barriers to activation of functional cellular immunity. In several embodiments, antagonistic antibodies specific for inhibitory ligands on T cells including Cytotoxic-T-lymphocyte-associated antigen 4 (CTLA-4; also known as CD152) and programmed cell death protein 1 (PD1 or PDCD1 also known as CD279) are used to enhance immunotherapy.

In several embodiments, there is provided genetically modified T cells that are non-alloreactive and highly active. In several embodiments, the T cells are further modified such that certain immune checkpoint genes are inactivated, and the immune checkpoint proteins are thus not expressed by the T cell. In several embodiments, this is done in the absence of manipulation or disruption of the CD3z signaling domain (e.g., the TCRs are still able initiate T cell signaling).

In several embodiments, genetic inactivation of TCRalpha and/or TCRbeta coupled with inactivation of immune checkpoint genes in T lymphocytes derived from an allogeneic donor significantly reduces the risk of GVHD. In several embodiments, this is done by eliminating at least a portion of one or more of the substituent protein chains (alpha, beta, gamma, and/or delta) responsible for recognition of MHC disparities between donor and recipient cells. In several embodiments, this is done while still allowing for T cell proliferation and activity.

In some embodiments wherein allogeneic cells are administered, the receiving subject may receive some other adjunct treatment to support or otherwise enhance the function of the administered immune cells. In several embodiments, the subject may be pre-conditioned (e.g., with radiation or chemotherapy). In some embodiments, the adjunct treatment comprises administration of lymphocyte growth factors (such as IL-2).

Moreover, in several embodiments, editing can improve persistence of administered cells (whether NK cells, T cells, or otherwise) for example, by masking cells to the host immune response. In some cases, a recipient's immune cells will attack donor cells, especially from an allogeneic donor, known as Host vs. Graft disease (HvG). FIG. 8F shows a schematic representation of HvG, where the host T cells, with a native/functional TCR identify HLA on donor T and/or donor NK cells as non-self. In such cases, the host T-cell TCR binding to allogeneic cell HLA leads to elimination of allogeneic cells, thus reducing the persistence of the donor engineered NK/T cells. Regarding HvG, to prevent rejection of administered allogeneic T cells, the subject receiving the cells requires suppression of their immune system In several embodiments, glucocorticoids are used, and include, but are not limited to beclomethasone, betamethasone, budesonide, cortisone, dexamethasone, hydrocortisone, methylprednisolone, prednisolone, prednisone, triamcinolone, among others. Activation of the glucocorticoid receptor in recipient's own T cells alters expression of genes involved in the immune response and results in reduced levels of cytokine production, which translates to T cell anergy and interference with T cell activation (in the recipient). Other embodiments relate to administration of antibodies that can deplete certain types of the recipients immune cells. One such target is CD52, which is expressed at high levels on T and B lymphocytes and lower levels on monocytes while being absent on granulocytes and bone marrow precursors. Treatment or pre-treatment of the recipient with Alemtuzumab, a humanized monoclonal antibody directed against CD52, has been shown to induce a rapid depletion of circulating lymphocytes and monocytes, thus lessening the probability of HvG, given the reduction in recipient immune cells. Immunosuppressive drugs may limit the efficacy of administered allogeneic engineered T cells. Therefore, as disclosed herein, several embodiments relate to genetically engineered allogeneic donor cells that are resistant to immunosuppressive treatment. In several embodiments, as discussed in more detail below, immune cells, such as NK cells and/or T cells are edited (in addition to being engineered to express a CAR) to extend their persistence by avoiding cytotoxic responses from host immune cells. In several embodiments, gene editing to remove one or more HLA molecules from the allogeneic NK and/or T cells reduce elimination by host T-cells. In several embodiments, the allogeneic NK and/or T cells are edited to knock out one or more of beta-2 microglobulin (an HLA Class I molecule) and CIITA (an HLA Class II molecule). FIG. 8G schematically depicts this approach.

In some embodiments of mixed allogeneic cell therapy, the populations of engineered cells actually target one another, for example when the therapeutic cells are edited to remove HLA molecules in order to avoid HvG. Such editing of, for example CAR T cells can result in the vulnerability of the edited allogeneic CAR T cells to cytotoxic attack by the CAR NK cells as well as elimination by host NK cells. This is caused by the missing “self” inhibitory signals generally presented by KIR molecules. FIG. 8H schematically depicts this process. In several embodiments, gene editing can be used to knock in expression of one or more “masking” molecules which mask the allogeneic cells from the host immune system and from fratricide by other administered engineered cells. FIG. 8I schematically depicts this approach. In several embodiments, proteins can be expressed on the surface of the allogeneic cells to inhibit targeting by NKs (both engineered NKs and host NKs), which advantageously prolongs persistence of both allogeneic CAR-Ts and CAR-NKs. In several embodiments, gene editing is used to knock in CD47, expression of which effectively functions as a “don't eat me” signal. In several embodiments, gene editing is used to knock in expression of HLA-E. HLA-E binds to both the inhibiting and activating receptors NKG2A and NKG2C, respectively that exist on the surface of NK cells. However, NKG2A is expressed to a greater degree in most human NK cells, thus, in several embodiments, expression of HLA-E on engineered cells results in an inhibitory effect of NK cells (both host and donor) against such cells edited to (or naturally expressing) HLA-E. In addition, in several embodiments, one or more viral HLA homologs are knocked in such that they are expressed by the engineered NK and/or T cells, thus conferring on the cells the ability of viruses to evade the host immune system. In several embodiments, these approaches advantageously prolong persistence of both allogeneic CAR-Ts and CAR-NKs.

In several embodiments, genetic editing (whether knock out or knock in) of any of the target genes (e.g., CISH, TGFBR, TCR, B2M, CIISH, CD47, HLA-E, or any other target gene disclosed herein), is accomplished through targeted introduction of DNA breakage, and subsequent DNA repair mechanism. In several embodiments, double strand breaks of DNA are repaired by non-homologous end joining (NHEJ), wherein enzymes are used to directly join the DNA ends to one another to repair the break. In several embodiments, however, double strand breaks are repaired by homology directed repair (HDR), which is advantageously more accurate, thereby allowing sequence specific breaks and repair. HDR uses a homologous sequence as a template for regeneration of missing DNA sequences at the break point, such as a vector with the desired genetic elements (e.g., an insertion element to disrupt the coding sequence of a TCR) within a sequence that is homologous to the flanking sequences of a double strand break. This will result in the desired change (e.g., insertion) being inserted at the site of the DSB.

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more of the TCR subunits.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in the TCR, or CISH, or any other target gene disclosed herein. Target sites in the TCR can readily be identified. Further information of target sites within a region of the TCR can be found in US Patent Publication No. 2018/0325955, and US Patent Publication No. 2015/0017136, each of which is incorporated by reference herein in its entirety. In several embodiments, two or more meganucleases, or functions fragments thereof, are fused to create a hybrid enzymes that recognize a desired target sequence within the target gene (e.g., CISH).

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ˜3 base pairs), in several embodiments, combinations of 2, 3, 4, 5, 6, 7, 8, 9, 10 or more zinc fingers whose recognition sites have been characterized are used, thereby allowing targeting of specific sequences, such as a portion of the TCR (or an immune checkpoint inhibitor). The combined ZFNs are then fused with the catalytic domain(s) of an endonuclease, such as FokI (optionally a FokI heterodimer), in order to induce a targeted DNA break. Additional information on uses of ZFNs to edit the TCR and/or immune checkpoint inhibitors can be found in U.S. Pat. No. 9,597,357, which is incorporated by reference herein.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein. In several embodiments, CRISPR is used to manipulate the gene(s) encoding a target gene to be knocked out or knocked in, for example CISH, TGFBR2, TCR, B2M, CIITA, CD47, HLA-E, etc. In several embodiments, CRISPR is used to edit one or more of the TCRs of a T cell and/or the genes encoding one or more immune checkpoint inhibitors. In several embodiments, the immune checkpoint inhibitor is selected from one or more of CTLA4 and PD1. In several embodiments, CRISPR is used to truncate one or more of TCRα, TCRβ, TCRγ, and TCRδ. In several embodiments, a TCR is truncated without impacting the function of the CD3z signaling domain of the TCR. Depending on the embodiment and which target gene is to be edited, a Class 1 or Class 2 Cas is used. In several embodiments, a Class 1 Cas is used and the Cas type is selected from the following types: I, IA, IB, IC, ID, IE, IF, IU, III, IIIA, IIIB, IIIC, IIID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, a Class 2 Cas is used and the Cas type is selected from the following types: II, IIA, IIB, IIC, V, VI, and combinations thereof. In several embodiments, the Cas is selected from the group consisting of Cas9, Csn2, Cas4, Cpf1, C2c1, C2c3, Cas13a (previously known as C2c2), Cas13b, Cas13c, and combinations thereof.

In several embodiments, as discussed above, editing of CISH advantageously imparts to the edited cells, particularly edited NK cells, enhanced expansion, cytotoxicity and/or persistence. Additionally, in several embodiments, the modification of the TCR comprises a modification to TCRα, but without impacting the signaling through the CD3 complex, allowing for T cell proliferation. In one embodiment, the TCRα is inactivated by expression of pre-Ta in the cells, thus restoring a functional CD3 complex in the absence of a functional alpha/beta TCR. As disclosed herein, the non-alloreactive modified T cells are also engineered to express a CAR to redirect the non-alloreactive T cells specificity towards tumor marker, but independent of MHC. Combinations of editing are used in several embodiments, such as knockout of the TCR and CISH in combination, or knock out of CISH and knock in of CD47, by way of non-limiting examples.

### Extracellular Domains (Tumor Binder)

Some embodiments of the compositions and methods described herein relate to a chimeric antigen receptor that includes an extracellular domain that comprises a tumor-binding domain (also referred to as an antigen-binding protein or antigen-binding domain) as described herein. The tumor binding domain, depending on the embodiment, targets, for example CD19, CD123, CD70, Her2, mesothelin, Claudin 6, BCMA, EGFR, among others. Several embodiments of the compositions and methods described herein relate to a chimeric receptor that includes an extracellular domain that comprises a ligand binding domain that binds a ligand expressed by a tumor cell (also referred to as an activating chimeric receptor) as described herein. The ligand binding domain, depending on the embodiment, targets for example MICA, MICB, ULBP1, ULBP2, ULBP3, ULBP4, ULBP5, and ULBP6 (among others).

In some embodiments, the antigen-binding domain is derived from or comprises wild-type or non-wild-type sequence of an antibody, an antibody fragment, an scFv, a Fv, a Fab, a (Fab′)2, a single domain antibody (SDAB), a vH or vL domain, a camelid VHH domain, or a non-immunoglobulin scaffold such as a DARPIN, an affibody, an affilin, an adnectin, an affitin, a repebody, a fynomer, an alphabody, an avimer, an atrimer, a centyrin, a pronectin, an anticalin, a kunitz domain, an Armadillo repeat protein, an autoantigen, a receptor or a ligand. In some embodiments, the tumor-binding domain contains more than one antigen binding domain. In embodiments, the antigen-binding domain is operably linked directly or via an optional linker to the NH2-terminal end of a TCR domain (e.g. constant chains of TCR-alpha, TCR-betal, TCR-beta2, preTCR-alpha, pre-TCR-alpha-Del48, TCR-gamma, or TCR-delta).

Antigen-Binding Proteins

There are provided, in several embodiments, antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is a cancer antigen (e.g., CD19) or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Nonlimiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact (e.g., native) antibody and the fragments may be produced by the modification of intact antibodies (e.g. enzymatic or chemical cleavage) or synthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain”, refers to the smaller of the two types of polypeptide chains present in antibody molecules in their naturally occurring conformations. Kappa (K) and lambda (A) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (μ), delta (A), gamma (γ), alpha (a), and epsilon (E), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4).

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the antigen-binding protein is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3) (see U.S. Pat. No. 6,703,199, which describes fibronectin polypeptide mini bodies). An antibody fragment may include a Fab, Fab′, F(ab′)2, and/or Fv fragment that contains at least one CDR of an immunoglobulin that is sufficient to confer specific antigen binding to a cancer antigen (e.g., CD19). Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, Md.), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). One or more CDRs may be incorporated into a molecule either covalently or noncovalently to make it an antigen-binding protein.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

There is also provided, in some embodiments, antigen-binding proteins with more than one binding site. In several embodiments, the binding sites are identical to one another while in some embodiments the binding sites are different from one another. For example, an antibody typically has two identical binding sites, while a “bispecific” or “bifunctional” antibody has two different binding sites. The two binding sites of a bispecific antigen-binding protein or antibody will bind to two different epitopes, which can reside on the same or different protein targets. In several embodiments, this is particularly advantageous, as a bispecific chimeric antigen receptor can impart to an engineered cell the ability to target multiple tumor markers. For example, CD19 and an additional tumor marker, such as CD123, CD70, Her2, mesothelin, Claudin 6, BCMA, EGFR, MICA, MICB, ULBP1, ULBP2, ULBP3, ULBP4, ULBP5, and ULBP6, among others, or any other marker disclosed herein or appreciated in the art as a tumor specific antigen or tumor associated antigen can be bound by a bispecific antibody.

As used herein, the term “chimeric antibody” shall be given its ordinary meaning, and shall also refer to an antibody that contains one or more regions from one antibody and one or more regions from one or more other antibodies. In some embodiments, one or more of the CDRs are derived from an anti-cancer antigen (e.g., CD19, CD123, CD70, Her2, mesothelin, PD-L1, Claudin 6, BCMA, EGFR, etc.) antibody. In several embodiments, all of the CDRs are derived from an anti-cancer antigen antibody (such as an anti-CD19 antibody). In some embodiments, the CDRs from more than one anti-cancer antigen antibodies are mixed and matched in a chimeric antibody. For instance, a chimeric antibody may comprise a CDR1 from the light chain of a first anti-cancer antigen antibody, a CDR2 and a CDR3 from the light chain of a second anti-cancer antigen antibody, and the CDRs from the heavy chain from a third anti-cancer antigen antibody. Further, the framework regions of antigen-binding proteins disclosed herein may be derived from one of the same anti-cancer antigen (e.g., CD19, CD123, CD70, Her2, mesothelin, Claudin 6, BCMA, EGFR, etc.) antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also provided herein are fragments of such antibodies that exhibit the desired biological activity.

In some embodiments, an antigen-binding protein is provided comprising a heavy chain variable domain having at least 90% identity to the VH domain amino acid sequence set forth in SEQ ID NO: 33. In some embodiments, the antigen-binding protein comprises a heavy chain variable domain having at least 95% identity to the VH domain amino acid sequence set forth in SEQ ID NO: 33. In some embodiments, the antigen-binding protein comprises a heavy chain variable domain having at least 96, 97, 98, or 99% identity to the VH domain amino acid sequence set forth in SEQ ID NO: 33. In several embodiments, the heavy chain variable domain may have one or more additional mutations (e.g., for purposes of humanization) in the VH domain amino acid sequence set forth in SEQ ID NO: 33, but retains specific binding to a cancer antigen (e.g., CD19). In several embodiments, the heavy chain variable domain may have one or more additional mutations in the VH domain amino acid sequence set forth in SEQ ID NO: 33, but has improved specific binding to a cancer antigen (e.g., CD19).

In some embodiments, the antigen-binding protein comprises a light chain variable domain having at least 90% identity to the VL domain amino acid sequence set forth in SEQ ID NO: 32. In some embodiments, the antigen-binding protein comprises a light chain variable domain having at least 95% identity to the VL domain amino acid sequence set forth in SEQ ID NO: 32. In some embodiments, the antigen-binding protein comprises a light chain variable domain having at least 96, 97, 98, or 99% identity to the VL domain amino acid sequence set forth in SEQ ID NO: 32. In several embodiments, the light chain variable domain may have one or more additional mutations (e.g., for purposes of humanization) in the VL domain amino acid sequence set forth in SEQ ID NO: 32, but retains specific binding to a cancer antigen (e.g., CD19). In several embodiments, the light chain variable domain may have one or more additional mutations in the VL domain amino acid sequence set forth in SEQ ID NO: 32, but has improved specific binding to a cancer antigen (e.g., CD19).

In some embodiments, the antigen-binding protein comprises a heavy chain variable domain having at least 90% identity to the VH domain amino acid sequence set forth in SEQ ID NO: 33, and a light chain variable domain having at least 90% identity to the VL domain amino acid sequence set forth in SEQ ID NO: 32. In some embodiments, the antigen-binding protein comprises a heavy chain variable domain having at least 95% identity to the VH domain amino acid sequence set forth in SEQ ID NO: 33, and a light chain variable domain having at least 95% identity to the VL domain amino acid sequence set forth in SEQ ID NO: 32. In some embodiments, the antigen-binding protein comprises a heavy chain variable domain having at least 96, 97, 98, or 99% identity to the VH domain amino acid sequence set forth in SEQ ID NO: 33, and a light chain variable domain having at least 96, 97, 98, or 99% identity to the VL domain amino acid sequence set forth in SEQ ID NO: 32.

In some embodiments, the antigen-binding protein comprises a heavy chain variable domain having the VH domain amino acid sequence set forth in SEQ ID NO: 33, and a light chain variable domain having the VL domain amino acid sequence set forth in SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a light chain variable domain of SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with SEQ ID NO: 33.

In some embodiments, the light chain variable domain comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the polynucleotide sequence SEQ ID NO: 32. In some embodiments, the light chain variable domain comprises a sequence of amino acids that is encoded by a polynucleotide that hybridizes under moderately stringent conditions to the complement of a polynucleotide that encodes a light chain variable domain in accordance with the sequence in SEQ ID NO: 32. In some embodiments, the light chain variable domain comprises a sequence of amino acids that is encoded by a polynucleotide that hybridizes under stringent conditions to the complement of a polynucleotide that encodes a light chain variable domain in accordance with the sequence in SEQ ID NO: 32.

In some embodiments, the heavy chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with the sequence of SEQ ID NO: 33. In some embodiments, the heavy chain variable domain comprises a sequence of amino acids that is encoded by a polynucleotide that hybridizes under moderately stringent conditions to the complement of a polynucleotide that encodes a heavy chain variable domain in accordance with the sequence of SEQ ID NO: 33. In some embodiments, the heavy chain variable domain comprises a sequence of amino acids that is encoded by a polynucleotide that hybridizes under stringent conditions to the complement of a polynucleotide that encodes a heavy chain variable domain in accordance with the sequence of SEQ ID NO: 33.

In several embodiments, additional anti-CD19 binding constructs are provided. For example, in several embodiments, there is provided an scFv that targets CD19 wherein the scFv comprises a heavy chain variable region comprising the sequence of SEQ ID NO. 35. In some embodiments, the antigen-binding protein comprises a heavy chain variable domain having at least 95% identity to the HCV domain amino acid sequence set forth in SEQ ID NO: 35. In some embodiments, the antigen-binding protein comprises a heavy chain variable domain having at least 96, 97, 98, or 99% identity to the HCV domain amino acid sequence set forth in SEQ ID NO: 35. In several embodiments, the heavy chain variable domain may have one or more additional mutations (e.g., for purposes of humanization) in the HCV domain amino acid sequence set forth in SEQ ID NO: 35, but retains specific binding to a cancer antigen (e.g., CD19). In several embodiments, the heavy chain variable domain may have one or more additional mutations in the HCV domain amino acid sequence set forth in SEQ ID NO: 35, but has improved specific binding to a cancer antigen (e.g., CD19).

Additionally, in several embodiments, an scFv that targets CD19 comprises a light chain variable region comprising the sequence of SEQ ID NO. 36. In some embodiments, the antigen-binding protein comprises a light chain variable domain having at least 95% identity to the LCV domain amino acid sequence set forth in SEQ ID NO: 36. In some embodiments, the antigen-binding protein comprises a light chain variable domain having at least 96, 97, 98, or 99% identity to the LCV domain amino acid sequence set forth in SEQ ID NO: 36. In several embodiments, the light chain variable domain may have one or more additional mutations (e.g., for purposes of humanization) in the LCV domain amino acid sequence set forth in SEQ ID NO: 36, but retains specific binding to a cancer antigen (e.g., CD19). In several embodiments, the light chain variable domain may have one or more additional mutations in the LCV domain amino acid sequence set forth in SEQ ID NO: 36, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 37. In several embodiments, the LC CDR1 comprises an amino acid sequence with at least about 85%, about 90%, about 95%, or about 98% sequence identity to the sequence of SEQ NO. 37. In several embodiments, the LC CDR2 comprises the sequence of SEQ ID NO. 38. In several embodiments, the LC CDR2 comprises an amino acid sequence with at least about 85%, about 90%, about 95%, or about 98% sequence identity to the sequence of SEQ NO. 38. In several embodiments, the LC CDR3 comprises the sequence of SEQ ID NO. 39. In several embodiments, the LC CDR3 comprises an amino acid sequence with at least about 85%, about 90%, about 95%, or about 98% sequence identity to the sequence of SEQ NO. 39. In several embodiments, the HC CDR1 comprises the sequence of SEQ ID NO. 40. In several embodiments, the HC CDR1 comprises an amino acid sequence with at least about 85%, about 90%, about 95%, or about 98% sequence identity to the sequence of SEQ NO. 40. In several embodiments, the HC CDR2 comprises the sequence of SEQ ID NO. 41, 42, or 43. In several embodiments, the HC CDR2 comprises an amino acid sequence with at least about 85%, about 90%, about 95%, or about 98% sequence identity to the sequence of SEQ NO. 41, 42, or 43. In several embodiments, the HC CDR3 comprises the sequence of SEQ ID NO. 44. In several embodiments, the HC CDR3 comprises an amino acid sequence with at least about 85%, about 90%, about 95%, or about 98% sequence identity to the sequence of SEQ NO. 44.

In several embodiments, there is also provided an anti-CD19 binding moiety that comprises a light chain variable region (VL) and a heavy chain variable region (HL), the VL region comprising a first, second and third complementarity determining region (VL CDR1, VL CDR2, and VL CDR3, respectively and the VH region comprising a first, second and third complementarity determining region (VH CDR1, VH CDR2, and VH CDR3, respectively. In several embodiments, the VL region comprises the sequence of SEQ ID NO. 45, 46, 47, or 48. In several embodiments, the VL region comprises an amino acid sequence with at least about 85%, about 90%, about 95%, or about 98% sequence identity to the sequence of SEQ NO. 45, 46, 47, or 48. In several embodiments, the VH region comprises the sequence of SEQ ID NO. 49, 50, 51 or 52. In several embodiments, the VH region comprises an amino acid sequence with at least about 85%, about 90%, about 95%, or about 98% sequence identity to the sequence of SEQ NO. 49, 50, 51 or 52.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 53. In several embodiments, the LC CDR1 comprises an amino acid sequence with at least about 85%, about 90%, about 95%, or about 98% sequence identity to the sequence of SEQ NO. 53. In several embodiments, the LC CDR2 comprises the sequence of SEQ ID NO. 54. In several embodiments, the LC CDR2 comprises an amino acid sequence with at least about 85%, about 90%, about 95%, or about 98% sequence identity to the sequence of SEQ NO. 54. In several embodiments, the LC CDR3 comprises the sequence of SEQ ID NO. 55. In several embodiments, the LC CDR3 comprises an amino acid sequence with at least about 85%, about 90%, about 95%, or about 98% sequence identity to the sequence of SEQ NO. 55. In several embodiments, the HC CDR1 comprises the sequence of SEQ ID NO. 56. In several embodiments, the HC CDR1 comprises an amino acid sequence with at least about 85%, about 90%, about 95%, or about 98% sequence identity to the sequence of SEQ NO. 56. In several embodiments, the HC CDR2 comprises the sequence of SEQ ID NO. 57. In several embodiments, the HC CDR2 comprises an amino acid sequence with at least about 85%, about 90%, about 95%, or about 98% sequence identity to the sequence of SEQ NO. 57. In several embodiments, the HC CDR3 comprises the sequence of SEQ ID NO. 58. In several embodiments, the HC CDR3 comprises an amino acid sequence with at least about 85%, about 90%, about 95%, or about 98% sequence identity to the sequence of SEQ NO. 58.

In some embodiments, the antigen-binding protein comprises a heavy chain variable region comprising the amino acid sequence of SEQ ID NO: 104. In some embodiments, the antigen-binding protein comprises a heavy chain variable region having at least 90% identity to the VH domain amino acid sequence set forth in SEQ ID NO: 104. In some embodiments, the antigen-binding protein comprises a heavy chain variable domain having at least 95% sequence identity to the VH domain amino acid sequence set forth in SEQ ID NO: 104. In some embodiments, the antigen-binding protein comprises a heavy chain variable domain having at least 96, 97, 98, or 99% sequence identity to the VH domain amino acid sequence set forth in SEQ ID NO: 104. In several embodiments, the heavy chain variable domain may have one or more additional mutations (e.g., for purposes of humanization) in the VH domain amino acid sequence set forth in SEQ ID NO: 104, but retains specific binding to a cancer antigen (e.g., CD19). In several embodiments, the heavy chain variable domain may have one or more additional mutations in the VH domain amino acid sequence set forth in SEQ ID NO: 104, but has improved specific binding to a cancer antigen (e.g., CD19).

In some embodiments, the antigen-binding protein comprises a light chain variable region comprising the amino acid sequence of SEQ ID NO: 105. In some embodiments, the antigen-binding protein comprises a light chain variable region having at least 90% sequence identity to the VL domain amino acid sequence set forth in SEQ ID NO: 105. In some embodiments, the antigen-binding protein comprises a light chain variable domain having at least 95% sequence identity to the VL domain amino acid sequence set forth in SEQ ID NO: 105. In some embodiments, the antigen-binding protein comprises a light chain variable domain having at least 96, 97, 98, or 99% sequence identity to the VL domain amino acid sequence set forth in SEQ ID NO: 105. In several embodiments, the light chain variable domain may have one or more additional mutations (e.g., for purposes of humanization) in the VL domain amino acid sequence set forth in SEQ ID NO: 105, but retains specific binding to a cancer antigen (e.g., CD19). In several embodiments, the light chain variable domain may have one or more additional mutations in the VL domain amino acid sequence set forth in SEQ ID NO: 105, but has improved specific binding to a cancer antigen (e.g., CD19).

In some embodiments, the antigen-binding protein comprises a heavy chain variable domain having the VH domain amino acid sequence set forth in SEQ ID NO: 104, and a light chain variable domain having the VL domain amino acid sequence set forth in SEQ ID NO: 105. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a light chain variable domain of SEQ ID NO: 105. In some embodiments, the heavy-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with SEQ ID NO: 104.

In some embodiments, the antigen-binding protein comprises a heavy chain variable comprising the amino acid sequence of SEQ ID NO: 106. In some embodiments, the antigen-binding protein comprises a heavy chain variable having at least 90% sequence identity to the VH amino acid sequence set forth in SEQ ID NO: 106. In some embodiments, the antigen-binding protein comprises a heavy chain variable having at least 95% sequence identity to the VH amino acid sequence set forth in SEQ ID NO: 106. In some embodiments, the antigen-binding protein comprises a heavy chain variable having at least 96, 97, 98, or 99% identity to the VH amino acid sequence set forth in SEQ ID NO: 106. In several embodiments, the heavy chain variable may have one or more additional mutations (e.g., for purposes of humanization) in the VH amino acid sequence set forth in SEQ ID NO: 106, but retains specific binding to a cancer antigen (e.g., CD19). In several embodiments, the heavy chain variable may have one or more additional mutations in the VH amino acid sequence set forth in SEQ ID NO: 106, but has improved specific binding to a cancer antigen (e.g., CD19).

In some embodiments, the antigen-binding protein comprises a light chain variable comprising the amino acid sequence of SEQ ID NO: 107. In some embodiments, the antigen-binding protein comprises a light chain variable region having at least 90% sequence identity to the VL amino acid sequence set forth in SEQ ID NO: 107. In some embodiments, the antigen-binding protein comprises a light chain variable having at least 95% sequence identity to the VL amino acid sequence set forth in SEQ ID NO: 107. In some embodiments, the antigen-binding protein comprises a light chain variable having at least 96, 97, 98, or 99% identity to the VL amino acid sequence set forth in SEQ ID NO: 107. In several embodiments, the light chain variable may have one or more additional mutations (e.g., for purposes of humanization) in the VL amino acid sequence set forth in SEQ ID NO: 107, but retains specific binding to a cancer antigen (e.g., CD19). In several embodiments, the light chain variable may have one or more additional mutations in the VL amino acid sequence set forth in SEQ ID NO: 107, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 108. In several embodiments, the LC CDR1 comprises an amino acid sequence with at least about 85%, about 90%, about 95%, or about 98% sequence identity to the sequence of SEQ NO. 108. In several embodiments, the LC CDR2 comprises the sequence of SEQ ID NO. 109. In several embodiments, the LC CDR2 comprises an amino acid sequence with at least about 85%, about 90%, about 95%, or about 98% sequence identity to the sequence of SEQ NO. 109. In several embodiments, the LC CDR3 comprises the sequence of SEQ ID NO. 110. In several embodiments, the LC CDR3 comprises an amino acid sequence with at least about 85%, about 90%, about 95%, or about 98% sequence identity to the sequence of SEQ NO. 110. In several embodiments, the HC CDR1 comprises the sequence of SEQ ID NO. 111. In several embodiments, the HC CDR1 comprises an amino acid sequence with at least about 85%, about 90%, about 95%, or about 98% sequence identity to the sequence of SEQ NO. 111. In several embodiments, the HC CDR2 comprises the sequence of SEQ ID NO. 112, 113, or 114. In several embodiments, the HC CDR2 comprises an amino acid sequence with at least about 85%, about 90%, about 95%, or about 98% sequence identity to the sequence of SEQ NO. 112, 113, or 114. In several embodiments, the HC CDR3 comprises the sequence of SEQ ID NO. 115. In several embodiments, the HC CDR3 comprises an amino acid sequence with at least about 85%, about 90%, about 95%, or about 98% sequence identity to the sequence of SEQ NO. 115. In several embodiments, the anti-CD19 binding moiety comprises SEQ ID NO: 116, or is sequence with at least about 85%, about 90%, about 95%, or about 98% sequence identity to the sequence of SEQ NO. 116.

In some embodiments, the antigen-binding protein comprises a light chain variable comprising the amino acid sequence of SEQ ID NO: 117, 118, or 119. In some embodiments, the antigen-binding protein comprises a light chain variable region having at least 90% identity to the VL amino acid sequence set forth in SEQ ID NO: 117, 118, or 119. In some embodiments, the antigen-binding protein comprises a light chain variable having at least 95% identity to the VL amino acid sequence set forth in SEQ ID NO: 117, 118, or 119. In some embodiments, the antigen-binding protein comprises a light chain variable having at least 96, 97, 98, or 99% identity to the VL amino acid sequence set forth in SEQ ID NO: 117, 118, or 119. In several embodiments, the light chain variable may have one or more additional mutations (e.g., for purposes of humanization) in the VL amino acid sequence set forth in SEQ ID NO: 117, 118, or 119, but retains specific binding to a cancer antigen (e.g., CD19). In several embodiments, the light chain variable may have one or more additional mutations in the VL amino acid sequence set forth in SEQ ID NO: 117, 118, or 119, but has improved specific binding to a cancer antigen (e.g., CD19).

In some embodiments, the antigen-binding protein comprises a heavy chain variable comprising the amino acid sequence of SEQ ID NO: 120, 121, 122, or 123. In some embodiments, the antigen-binding protein comprises a heavy chain variable having at least 90% identity to the VH amino acid sequence set forth in SEQ ID NO: 120, 121, 122, or 123. In some embodiments, the antigen-binding protein comprises a heavy chain variable having at least 95% identity to the VH amino acid sequence set forth in SEQ ID NO: 120, 121, 122, or 123. In some embodiments, the antigen-binding protein comprises a heavy chain variable having at least 96, 97, 98, or 99% identity to the VH amino acid sequence set forth in SEQ ID NO: 120, 121, 122, or 123. In several embodiments, the heavy chain variable may have one or more additional mutations (e.g., for purposes of humanization) in the VH amino acid sequence set forth in SEQ ID NO: 120, 121, 122, or 123, but retains specific binding to a cancer antigen (e.g., CD19). In several embodiments, the heavy chain variable may have one or more additional mutations in the VH amino acid sequence set forth in SEQ ID NO: 120, 121, 122, or 123, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 124, 127, or 130. In several embodiments, the LC CDR1 comprises an amino acid sequence with at least about 85%, about 90%, about 95%, or about 98% sequence identity to the sequence of SEQ NO. 124, 127, or 130. In several embodiments, the LC CDR2 comprises the sequence of SEQ ID NO. 125, 128, or 131. In several embodiments, the LC CDR2 comprises an amino acid sequence with at least about 85%, about 90%, about 95%, or about 98% sequence identity to the sequence of SEQ NO. 125, 128, or 131. In several embodiments, the LC CDR3 comprises the sequence of SEQ ID NO. 126, 129, or 132. In several embodiments, the LC CDR3 comprises an amino acid sequence with at least about 85%, about 90%, about 95%, or about 98% sequence identity to the sequence of SEQ NO. 126, 129, or 132. In several embodiments, the HC CDR1 comprises the sequence of SEQ ID NO. 133, 136, 139, or 142. In several embodiments, the HC CDR1 comprises an amino acid sequence with at least about 85%, about 90%, about 95%, or about 98% sequence identity to the sequence of SEQ NO. 133, 136, 139, or 142. In several embodiments, the HC CDR2 comprises the sequence of SEQ ID NO. 134, 137, 140, or 143. In several embodiments, the HC CDR2 comprises an amino acid sequence with at least about 85%, about 90%, about 95%, or about 98% sequence identity to the sequence of SEQ NO. 134, 137, 140, or 143. In several embodiments, the HC CDR3 comprises the sequence of SEQ ID NO. 135, 138, 141, or 144. In several embodiments, the HC CDR3 comprises an amino acid sequence with at least about 85%, about 90%, about 95%, or about 98% sequence identity to the sequence of SEQ NO. 135, 138, 141, or 144.

Additional anti-CD19 binding moieties are known in the art, such as those disclosed in, for example, U.S. Pat. No. 8,399,645, US Patent Publication No. 2018/0153977, US Patent Publication No. 2014/0271635, US Patent Publication No. 2018/0251514, and US Patent Publication No. 2018/0312588, the entirety of each of which is incorporated by reference herein.

Several embodiments relate to CARs that are directed to Claudin 6, and show little or no binding to Claudin 3, 4, or 9 (or other Claudins). In some embodiments, the antigen-binding protein comprises a heavy chain variable comprising the amino acid sequence of SEQ ID NO: 88. In some embodiments, the antigen-binding protein comprises a heavy chain variable having at least 90% identity to the VH amino acid sequence set forth in SEQ ID NO: 88. In some embodiments, the antigen-binding protein comprises a heavy chain variable having at least 95% identity to the VH amino acid sequence set forth in SEQ ID NO: 88. In some embodiments, the antigen-binding protein comprises a heavy chain variable having at least 96, 97, 98, or 99% identity to the VH amino acid sequence set forth in SEQ ID NO: 88. In several embodiments, the heavy chain variable may have one or more additional mutations (e.g., for purposes of humanization) in the VH amino acid sequence set forth in SEQ ID NO: 88, but retains specific binding to a cancer antigen (e.g., CLDN6). In several embodiments, the heavy chain variable may have one or more additional mutations in the VH amino acid sequence set forth in SEQ ID NO: 88, but has improved specific binding to a cancer antigen (e.g., CLDN6).

In some embodiments, the antigen-binding protein comprises a light chain variable comprising the amino acid sequence of SEQ ID NO: 89, 90 or 91. In some embodiments, the antigen-binding protein comprises a light chain variable region having at least 90% identity to the VL amino acid sequence set forth in SEQ ID NO: 89, 90 or 91. In some embodiments, the antigen-binding protein comprises a light chain variable having at least 95% identity to the VL amino acid sequence set forth in SEQ ID NO: 89, 90 or 91. In some embodiments, the antigen-binding protein comprises a light chain variable having at least 96, 97, 98, or 99% identity to the VL amino acid sequence set forth in SEQ ID NO: 89, 90 or 91. In several embodiments, the light chain variable may have one or more additional mutations (e.g., for purposes of humanization) in the VL amino acid sequence set forth in SEQ ID NO: 89, 90 or 91, but retains specific binding to a cancer antigen (e.g., CLDN6). In several embodiments, the light chain variable may have one or more additional mutations in the VL amino acid sequence set forth in SEQ ID NO: 89, 90 or 91, but has improved specific binding to a cancer antigen (e.g., CLDN6).

In several embodiments, there is also provided an anti-CLDN6 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 95, 98, or 101. In several embodiments, the LC CDR1 comprises an amino acid sequence with at least about 85%, about 90%, about 95%, or about 98% sequence identity to the sequence of SEQ NO. 95, 98, or 101. In several embodiments, the LC CDR2 comprises the sequence of SEQ ID NO. 96, 99, or 102. In several embodiments, the LC CDR2 comprises an amino acid sequence with at least about 85%, about 90%, about 95%, or about 98% sequence identity to the sequence of SEQ NO. 96, 99, or 102. In several embodiments, the LC CDR3 comprises the sequence of SEQ ID NO. 97, 100, or 103. In several embodiments, the LC CDR3 comprises an amino acid sequence with at least about 85%, about 90%, about 95%, or about 98% sequence identity to the sequence of SEQ NO. 97, 100, or 103. In several embodiments, the HC CDR1 comprises the sequence of SEQ ID NO. 92. In several embodiments, the HC CDR1 comprises an amino acid sequence with at least about 85%, about 90%, about 95%, or about 98% sequence identity to the sequence of SEQ NO. 92. In several embodiments, the HC CDR2 comprises the sequence of SEQ ID NO. 93. In several embodiments, the HC CDR2 comprises an amino acid sequence with at least about 85%, about 90%, about 95%, or about 98% sequence identity to the sequence of SEQ NO. 93. In several embodiments, the HC CDR3 comprises the sequence of SEQ ID NO. 94. In several embodiments, the HC CDR3 comprises an amino acid sequence with at least about 85%, about 90%, about 95%, or about 98% sequence identity to the sequence of SEQ NO. 94. In several embodiments, the antigen-binding protein does not bind claudins other than CLDN6

Natural Killer Group Domains that Bind Tumor Ligands

In several embodiments, engineered immune cells such as NK cells are leveraged for their ability to recognize and destroy tumor cells. For example, an engineered NK cell may include a CD19-directed chimeric antigen receptor or a nucleic acid encoding said chimeric antigen receptor (or a CAR directed against, for example, one or more of CD123, CD70, Her2, mesothelin, Claudin 6, BCMA, EGFR, etc.). NK cells express both inhibitory and activating receptors on the cell surface. Inhibitory receptors bind self-molecules expressed on the surface of healthy cells (thus preventing immune responses against “self” cells), while the activating receptors bind ligands expressed on abnormal cells, such as tumor cells. When the balance between inhibitory and activating receptor activation is in favor of activating receptors, NK cell activation occurs and target (e.g., tumor) cells are lysed.

Natural killer Group 2 member D (NKG2D) is an NK cell activating receptor that recognizes a variety of ligands expressed on cells. The surface expression of various NKG2D ligands is generally low in healthy cells but is upregulated upon, for example, malignant transformation. Non-limiting examples of ligands recognized by NKG2D include, but are not limited to, MICA, MICB, ULBP1, ULBP2, ULBP3, ULBP4, ULBP5, and ULBP6, as well as other molecules expressed on target cells that control the cytolytic or cytotoxic function of NK cells. In several embodiments, T cells are engineered to express an extracellular domain to binds to one or more tumor ligands and activate the T cell. For example, in several embodiments, T cells are engineered to express an NKG2D receptor as the binder/activation moiety. In several embodiments, engineered cells as disclosed herein are engineered to express another member of the NKG2 family, e.g., NKG2A, NKG2C, and/or NKG2E. Combinations of such receptors are engineered in some embodiments. Moreover, in several embodiments, other receptors are expressed, such as the Killer-cell immunoglobulin-like receptors (KIRs).

In several embodiments, cells are engineered to express a cytotoxic receptor complex comprising a full length NKG2D as an extracellular component to recognize ligands on the surface of tumor cells (e.g., liver cells). In one embodiment, full length NKG2D has the nucleic acid sequence of SEQ ID NO: 27. In several embodiments, the full length NKG2D, or functional fragment thereof is human NKG2D. Additional information about chimeric receptors for use in the presently disclosed methods and compositions can be found in PCT Patent Publication No. WO/2018/183385, which is incorporated in its entirety by reference herein.

In several embodiments, cells are engineered to express a cytotoxic receptor complex comprising a functional fragment of NKG2D as an extracellular component to recognize ligands on the surface of tumor cells or other diseased cells. In one embodiment, the functional fragment of NKG2D has the nucleic acid sequence of SEQ ID NO: 25. In several embodiments, the fragment of NKG2D is at least 70%, at least 75%, at least 80%, at least 85%, at least 90%, or at least 95% homologous with full-length wild-type NKG2D. In several embodiments, the fragment may have one or more additional mutations from SEQ ID NO: 25, but retains, or in some embodiments, has enhanced, ligand-binding function. In several embodiments, the functional fragment of NKG2D comprises the amino acid sequence of SEQ ID NO: 26. In several embodiments, the NKG2D fragment is provided as a dimer, trimer, or other concatameric format, such embodiments providing enhanced ligand-binding activity. In several embodiments, the sequence encoding the NKG2D fragment is optionally fully or partially codon optimized. In one embodiment, a sequence encoding a codon optimized NKG2D fragment comprises the sequence of SEQ ID NO: 28. Advantageously, according to several embodiments, the functional fragment lacks its native transmembrane or intracellular domains but retains its ability to bind ligands of NKG2D as well as transduce activation signals upon ligand binding. A further advantage of such fragments is that expression of DAP10 to localize NKG2D to the cell membrane is not required. Thus, in several embodiments, the cytotoxic receptor complex encoded by the polypeptides disclosed herein does not comprise DAP10. In several embodiments, immune cells, such as NK or T cells (e.g., non-alloreactive T cells engineered according to embodiments disclosed herein), are engineered to express one or more chimeric receptors that target, for example CD19, CD123, CD70, Her2, mesothelin, Claudin 6, BCMA, EGFR, and an NKG2D ligand, such as MICA, MICB, ULBP1, ULBP2, ULBP3, ULBP4, ULBP5, and/or ULBP6. Such cells, in several embodiments, also co-express mbIL15.

In several embodiments, the cytotoxic receptor complexes are configured to dimerize. Dimerization may comprise homodimers or heterodimers, depending on the embodiment. In several embodiments, dimerization results in improved ligand recognition by the cytotoxic receptor complexes (and hence the NK cells expressing the receptor), resulting in a reduction in (or lack) of adverse toxic effects. In several embodiments, the cytotoxic receptor complexes employ internal dimers, or repeats of one or more component subunits. For example, in several embodiments, the cytotoxic receptor complexes may optionally comprise a first NKG2D extracellular domain coupled to a second NKG2D extracellular domain, and a transmembrane/signaling region (or a separate transmembrane region along with a separate signaling region).

In several embodiments, the various domains/subdomains are separated by a linker such as, a GS3 linker (SEQ ID NO: 15 and 16, nucleotide and protein, respectively) is used (or a GSn linker). Other linkers used according to various embodiments disclosed herein include, but are not limited to those encoded by SEQ ID NO: 17, 19, 21 or 23. This provides the potential to separate the various component parts of the receptor complex along the polynucleotide, which can enhance expression, stability, and/or functionality of the receptor complex.

### Cytotoxic Signaling Complex

Some embodiments of the compositions and methods described herein relate to a chimeric receptor, such as a chimeric antigen receptor (e.g., a CAR directed to CD19, CD123, CD70, Her2, mesothelin, Claudin 6, BCMA, or EGFR (among others), or a chimeric receptor directed against an NKG2D ligand, such as MICA, MICB, ULBP1, ULBP2, ULBP3, ULBP4, ULBP5, and/or ULBP6) that includes a cytotoxic signaling complex. As disclosed herein, according to several embodiments, the provided cytotoxic receptor complexes comprise one or more transmembrane and/or intracellular domains that initiate cytotoxic signaling cascades upon the extracellular domain(s) binding to ligands on the surface of target cells.

In several embodiments, the cytotoxic signaling complex comprises at least one transmembrane domain, at least one co-stimulatory domain, and/or at least one signaling domain. In some embodiments, more than one component part makes up a given domain—e.g., a co-stimulatory domain may comprise two subdomains. Moreover, in some embodiments, a domain may serve multiple functions, for example, a transmembrane domain may also serve to provide signaling function.

### Transmembrane Domains

Some embodiments of the compositions and methods described herein relate to chimeric receptors (e.g., tumor antigen-directed CARs and/or ligand-directed chimeric receptors) that comprise a transmembrane domain. Some embodiments include a transmembrane domain from NKG2D or another transmembrane protein. In several embodiments in which a transmembrane domain is employed, the portion of the transmembrane protein employed retains at least a portion of its normal transmembrane domain.

In several embodiments, however, the transmembrane domain comprises at least a portion of CD8, a transmembrane glycoprotein normally expressed on both T cells and NK cells. In several embodiments, the transmembrane domain comprises CD8a. In several embodiments, the transmembrane domain is referred to as a “hinge”. In several embodiments, the “hinge” of CD8a has the nucleic acid sequence of SEQ ID NO: 1. In several embodiments, the CD8a hinge is truncated or modified and is at least 70%, at least 75%, at least 80%, at least 85%, at least 90%, at least 95% homologous with the CD8a having the sequence of SEQ ID NO: 1. In several embodiments, the “hinge” of CD8a comprises the amino acid sequence of SEQ ID NO: 2. In several embodiments, the CD8a can be truncated or modified, such that it is at least 70%, at least 75%, at least 80%, at least 85%, at least 90%, at least 95% homologous with the sequence of SEQ ID NO: 2.

In several embodiments, the transmembrane domain comprises a CD8a transmembrane region. In several embodiments, the CD8a transmembrane domain has the nucleic acid sequence of SEQ ID NO: 3. In several embodiments, the CD8a hinge is truncated or modified and is at least 70%, at least 75%, at least 80%, at least 85%, at least 90%, at least 95% homologous with the CD8a having the sequence of SEQ ID NO: 3. In several embodiments, the CD8a transmembrane domain comprises the amino acid sequence of SEQ ID NO: 4. In several embodiments, the CD8a hinge is truncated or modified and is at least 70%, at least 75%, at least 80%, at least 85%, at least 90%, at least 95% homologous with the CD8a having the sequence of SEQ ID NO: 4.

Taken together in several embodiments, the CD8 hinge/transmembrane complex is encoded by the nucleic acid sequence of SEQ ID NO: 13. In several embodiments, the CD8 hinge/transmembrane complex is truncated or modified and is at least 70%, at least 75%, at least 80%, at least 85%, at least 90%, at least 95% homologous with the CD8 hinge/transmembrane complex having the sequence of SEQ ID NO: 13. In several embodiments, the CD8 hinge/transmembrane complex comprises the amino acid sequence of SEQ ID NO: 14. In several embodiments, the CD8 hinge/transmembrane complex hinge is truncated or modified and is at least 70%, at least 75%, at least 80%, at least 85%, at least 90%, at least 95% homologous with the CD8 hinge/transmembrane complex having the sequence of SEQ ID NO: 14.

In some embodiments, the transmembrane domain comprises a CD28 transmembrane domain or a fragment thereof. In several embodiments, the CD28 transmembrane domain comprises the amino acid sequence of SEQ ID NO: 30. In several embodiments, the CD28 transmembrane domain complex hinge is truncated or modified and is at least 70%, at least 75%, at least 80%, at least 85%, at least 90%, at least 95% homologous with the CD28 transmembrane domain having the sequence of SEQ ID NO: 30.

### Co-Stimulatory Domains

Some embodiments of the compositions and methods described herein relate to chimeric receptors (e.g., tumor antigen-directed CARs and/or tumor ligand-directed chimeric receptors) that comprise a co-stimulatory domain. In addition the various the transmembrane domains and signaling domain (and the combination transmembrane/signaling domains), additional co-activating molecules can be provided, in several embodiments. These can be certain molecules that, for example, further enhance activity of the immune cells. Cytokines may be used in some embodiments. For example, certain interleukins, such as IL-2 and/or IL-15 as non-limiting examples, are used. In some embodiments, the immune cells for therapy are engineered to express such molecules as a secreted form. In additional embodiments, such co-stimulatory domains are engineered to be membrane bound, acting as autocrine stimulatory molecules (or even as paracrine stimulators to neighboring cells). In several embodiments, NK cells are engineered to express membrane-bound interleukin 15 (mbIL15). In such embodiments, mbIL15 expression on the NK enhances the cytotoxic effects of the engineered NK cell by enhancing the proliferation and/or longevity of the NK cells. In several embodiments, T cells, such as the genetically engineered non-alloreactive T cells disclosed herein are engineered to express membrane-bound interleukin 15 (mbIL15). In such embodiments, mbIL15 expression on the T cell enhances the cytotoxic effects of the engineered T cell by enhancing the activity and/or propagation (e.g., longevity) of the engineered T cells. In several embodiments, mbIL15 has the nucleic acid sequence of SEQ ID NO: 11. In several embodiments, mbIL15 can be truncated or modified, such that it is at least 70%, at least 75%, at least 80%, at least 85%, at least 90%, at least 95% homologous with the sequence of SEQ ID NO: 11. In several embodiments, the mbIL15 comprises the amino acid sequence of SEQ ID NO: 12. In several embodiments, the mbIL15 is truncated or modified and is at least 70%, at least 75%, at least 80%, at least 85%, at least 90%, at least 95% homologous with the mbIL15 having the sequence of SEQ ID NO: 12.

In some embodiments, the tumor antigen-directed CARs and/or tumor ligand-directed chimeric receptors are encoded by a polynucleotide that includes one or more cytosolic protease cleavage sites, for example a T2A cleavage site, a P2A cleavage site, an E2A cleavage site, and/or a F2A cleavage site. Such sites are recognized and cleaved by a cytosolic protease, which can result in separation (and separate expression) of the various component parts of the receptor encoded by the polynucleotide. As a result, depending on the embodiment, the various constituent parts of an engineered cytotoxic receptor complex can be delivered to an NK cell or T cell in a single vector or by multiple vectors. Thus, as shown schematically, in the Figures, a construct can be encoded by a single polynucleotide, but also include a cleavage site, such that downstream elements of the constructs are expressed by the cells as a separate protein (as is the case in some embodiments with IL-15). In several embodiments, a T2A cleavage site is used. In several embodiments, a T2A cleavage site has the nucleic acid sequence of SEQ ID NO: 9. In several embodiments, T2A cleavage site can be truncated or modified, such that it is at least 70%, at least 75%, at least 80%, at least 85%, at least 90%, at least 95% homologous with the sequence of SEQ ID NO: 9. In several embodiments, the T2A cleavage site comprises the amino acid sequence of SEQ ID NO: 10. In several embodiments, the T2A cleavage site is truncated or modified and is at least 70%, at least 75%, at least 80%, at least 85%, at least 90%, at least 95% homologous with the T2A cleavage site having the sequence of SEQ ID NO: 10.

### Signaling Domains

Some embodiments of the compositions and methods described herein relate to a chimeric receptor (e.g., tumor antigen-directed CARs and/or tumor ligand-directed chimeric receptors) that includes a signaling domain. For example, immune cells engineered according to several embodiments disclosed herein may comprise at least one subunit of the CD3 T cell receptor complex (or a fragment thereof). In several embodiments, the signaling domain comprises the CD3 zeta subunit. In several embodiments, the CD3 zeta is encoded by the nucleic acid sequence of SEQ ID NO: 7. In several embodiments, the CD3 zeta can be truncated or modified, such that it is at least 70%, at least 75%, at least 80%, at least 85%, at least 90%, at least 95% homologous with the CD3 zeta having the sequence of SEQ ID NO: 7. In several embodiments, the CD3 zeta domain comprises the amino acid sequence of SEQ ID NO: 8. In several embodiments, the CD3 zeta domain is truncated or modified and is at least 70%, at least 75%, at least 80%, at least 85%, at least 90%, at least 95% homologous with the CD3 zeta domain having the sequence of SEQ ID NO: 8.

In several embodiments, unexpectedly enhanced signaling is achieved through the use of multiple signaling domains whose activities act synergistically. For example, in several embodiments, the signaling domain further comprises an OX40 domain. In several embodiments, the OX40 domain is an intracellular signaling domain. In several embodiments, the OX40 intracellular signaling domain has the nucleic acid sequence of SEQ ID NO: 5. In several embodiments, the OX40 intracellular signaling domain can be truncated or modified, such that it is at least 70%, at least 75%, at least 80%, at least 85%, at least 90%, at least 95% homologous with the OX40 having the sequence of SEQ ID NO: 5. In several embodiments, the OX40 intracellular signaling domain comprises the amino acid sequence of SEQ ID NO: 6. In several embodiments, the OX40 intracellular signaling domain is truncated or modified and is at least 70%, at least 75%, at least 80%, at least 85%, at least 90%, at least 95% homologous with the OX40 intracellular signaling domain having the sequence of SEQ ID NO: 6. In several embodiments, OX40 is used as the sole transmembrane/signaling domain in the construct, however, in several embodiments, OX40 can be used with one or more other domains. For example, combinations of OX40 and CD3zeta are used in some embodiments. By way of further example, combinations of CD28, OX40, 4-1 BB, and/or CD3zeta are used in some embodiments.

In several embodiments, the signaling domain comprises a 4-1 BB domain. In several embodiments, the 4-1 BB domain is an intracellular signaling domain. In several embodiments, the 4-1 BB intracellular signaling domain comprises the amino acid sequence of SEQ ID NO: 29. In several embodiments, the 4-1 BB intracellular signaling domain is truncated or modified and is at least 70%, at least 75%, at least 80%, at least 85%, at least 90%, at least 95% homologous with the 4-1BB intracellular signaling domain having the sequence of SEQ ID NO: 29. In several embodiments, 4-1 BB is used as the sole transmembrane/signaling domain in the construct, however, in several embodiments, 4-1BB can be used with one or more other domains. For example, combinations of 4-1 BB and CD3zeta are used in some embodiments. By way of further example, combinations of CD28, OX40, 4-1 BB, and/or CD3zeta are used in some embodiments.

In several embodiments, the signaling domain comprises a CD28 domain. In several embodiments the CD28 domain is an intracellular signaling domain. In several embodiments, the CD28 intracellular signaling domain comprises the amino acid sequence of SEQ ID NO: 31. In several embodiments, the CD28 intracellular signaling domain is truncated or modified and is at least 70%, at least 75%, at least 80%, at least 85%, at least 90%, at least 95% homologous with the CD28 intracellular signaling domain having the sequence of SEQ ID NO: 31. In several embodiments, CD28 is used as the sole transmembrane/signaling domain in the construct, however, in several embodiments, CD28 can be used with one or more other domains. For example, combinations of CD28 and CD3zeta are used in some embodiments. By way of further example, combinations of CD28, OX40, 4-1 BB, and/or CD3zeta are used in some embodiments.

### Cytotoxic Receptor Complex Constructs

Some embodiments of the compositions and methods described herein relate to chimeric antigen receptors, such as a CD19-directed chimeric receptor, as well as chimeric receptors, such as an activating chimeric receptor (ACR) that targets ligands of NKG2D. The expression of these cytotoxic receptors complexes in immune cells, such as genetically modified non-alloreactive T cells and/or NK cells, allows the targeting and destruction of particular target cells, such as cancerous cells. Non-limiting examples of such cytotoxic receptor complexes are discussed in more detail below.

### Chimeric Antigen Receptor Cytotoxic Receptor Complex Constructs

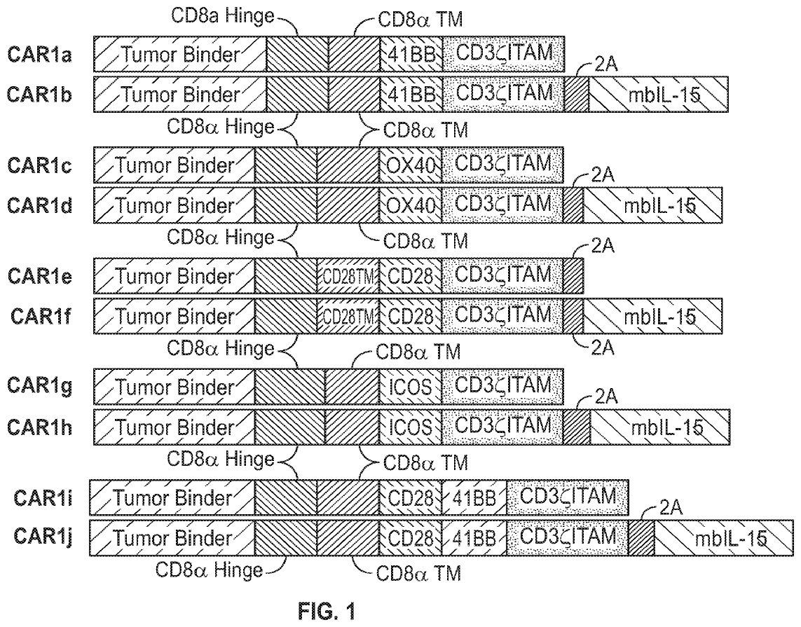

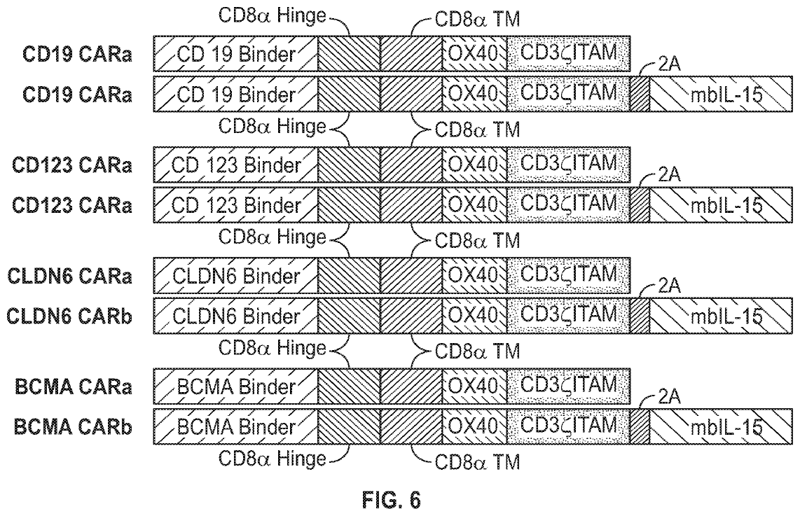

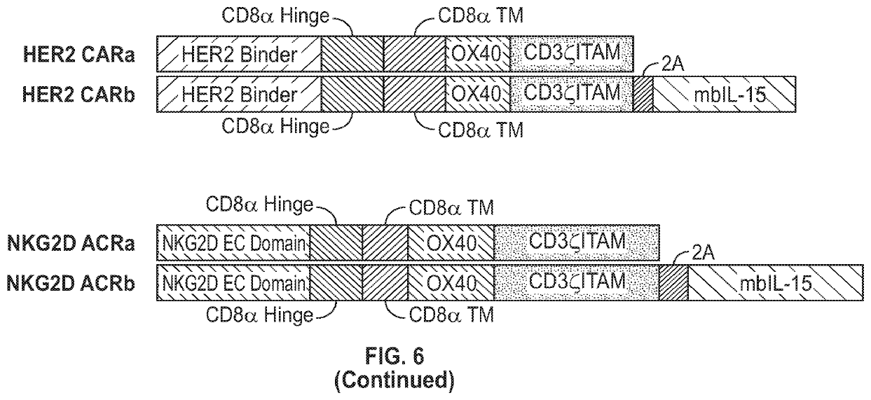

In several embodiments, there are provided for herein a variety of cytotoxic receptor complexes (also referred to as cytotoxic receptors) are provided for herein with the general structure of a chimeric antigen receptor. FIGS. 1-7 schematically depict non-limiting schematics of constructs that include an tumor binding moiety that binds to tumor antigens or tumor-associated antigens expressed on the surface of cancer cells and activates the engineered cell expressing the chimeric antigen receptor. FIG. 6 shows a schematic of a chimeric receptor complex, with an NKG2D activating chimeric receptor as a non-limiting example (see NKG2D ACRa and ACRb). FIG. 6 shows a schematic of a bispecific CAR/chimeric receptor complex, with an NKG2D activating chimeric receptor as a non-limiting example (see Bi-spec CAR/ACRa and CAR/ACRb).

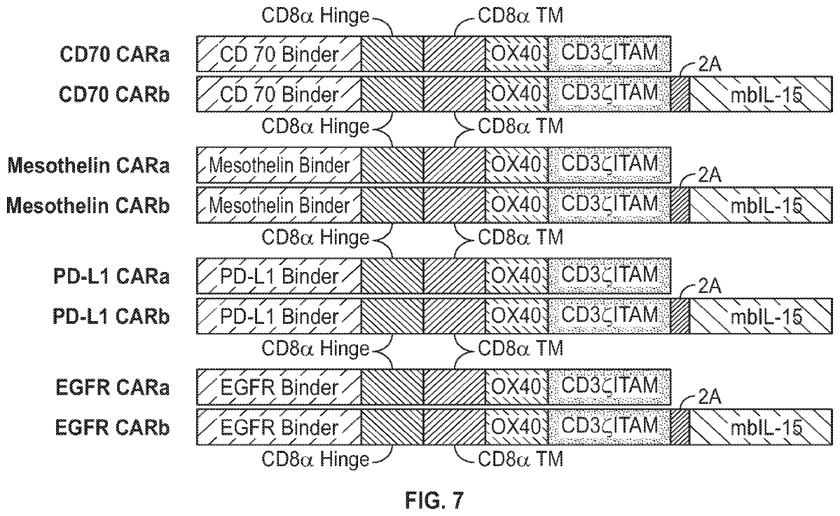

As shown in the figures, several embodiments of the chimeric receptor include an anti-tumor binder, a CD8a hinge domain, an Ig4 SH domain (or hinge), a CD8a transmembrane domain, a CD28 transmembrane domain, an OX40 domain, a 4-1BB domain, a CD28 domain, a CD3 ITAM domain or subdomain, a CD3zeta domain, an NKp80 domain, a CD16 IC domain, a 2A cleavage site, and a membrane-bound IL-15 domain (though, as above, in several embodiments soluble IL-15 is used). In several embodiments, the binding and activation functions are engineered to be performed by separate domains. Several embodiments relate to complexes with more than one tumor binder moiety or other binder/activation moiety. In some embodiments, the binder/activation moiety targets other markers besides CD19, such as a cancer target described herein. For example, FIGS. 6 and 7 depict schematics of non-limiting examples of CAR constructs that target different antigens, such as CD123, CLDN6, BCMA, HER2, CD70, Mesothelia, PD-L1, and EGFR. In several embodiments, the general structure of the chimeric antigen receptor construct includes a hinge and/or transmembrane domain. These may, in some embodiments, be fulfilled by a single domain, or a plurality of subdomains may be used, in several embodiments. The receptor complex further comprises a signaling domain, which transduces signals after binding of the homing moiety to the target cell, ultimately leading to the cytotoxic effects on the target cell. In several embodiments, the complex further comprises a co-stimulatory domain, which operates, synergistically, in several embodiments, to enhance the function of the signaling domain. Expression of these complexes in immune cells, such as T cells and/or NK cells, allows the targeting and destruction of particular target cells, such as cancerous cells that express a given tumor marker. Some such receptor complexes comprise an extracellular domain comprising an anti-CD19 moiety, or CD19-binding moiety, that binds CD19 on the surface of target cells and activates the engineered cell. The CD3zeta ITAM subdomain may act in concert as a signaling domain. The IL-15 domain, e.g., mbIL-15 domain, may act as a co-stimulatory domain. The IL-15 domain, e.g. mbIL-15 domain, may render immune cells (e.g., NK or T cells) expressing it particularly efficacious against target tumor cells. It shall be appreciated that the IL-15 domain, such as an mbIL-15 domain, can, in accordance with several embodiments, be encoded on a separate construct. Additionally, each of the components may be encoded in one or more separate constructs. In some embodiments, the cytotoxic receptor or CD19-directed receptor comprises a sequence of amino acids that is at least 70%, 75%, 80%, 85%, 90%, 91%, 92%, 93%, 94%, 95%, 96%, 97%, 98%, 99%, or more, or a range defined by any two of the aforementioned percentages, identical to the sequence of SEQ ID NO: 34.

Depending on the embodiment, various binders can be used to target CD19. In several embodiments, peptide binders are used, while in some embodiments antibodies, or fragments thereof are used. In several embodiments employing antibodies, antibody sequences are optimized, humanized or otherwise manipulated or mutated from their native form in order to increase one or more of stability, affinity, avidity or other characteristic of the antibody or fragment. In several embodiments, an antibody is provided that is specific for CD19. In several embodiments, an scFv is provided that is specific for CD19. In several embodiments, the antibody or scFv specific for CD19 comprises a heavy chain variable comprising the amino acid sequence of SEQ ID NO: 104 or 106. In some embodiments, the heavy chain variable comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 104 or 106. In some embodiments, the heavy chain variable comprises a sequence of amino acids that is encoded by a polynucleotide that hybridizes under moderately stringent conditions to the complement of a polynucleotide that encodes a heavy chain variable of SEQ ID NO. 104 or 106. In some embodiments, the heavy chain variable domain a sequence of amino acids that is encoded by a polynucleotide that hybridizes under stringent conditions to the complement of a polynucleotide that encodes a heavy chain variable encodes a heavy chain variable of SEQ ID NO. 104 or 106.

In several embodiments, the antibody or scFv specific for CD19 comprises a light chain variable comprising the amino acid sequence of any of SEQ ID NO. 105 or 107. In several embodiments, the light chain variable comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the identical to the sequence of SEQ ID NO. 105 or 107. In some embodiments, the light chain variable comprises a sequence of amino acids that is encoded by a polynucleotide that hybridizes under moderately stringent conditions to the complement of a polynucleotide that encodes a light chain variable of SEQ ID NO. 105 or 107. In some embodiments, the light chain variable domain comprises a sequence of amino acids that is encoded by a polynucleotide that hybridizes under stringent conditions to the complement of a polynucleotide that encodes a light chain variable domain of SEQ ID NO. 105 or 107.

In several embodiments, the anti-CD19 antibody or scFv comprises one, two, or three heavy chain complementarity determining region (CDR) and one, two, or three light chain CDRs. In several embodiments, a first heavy chain CDR has the amino acid sequence of SEQ ID NO: 111. In some embodiments, the first heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 111. In several embodiments, a second heavy chain CDR has the amino acid sequence of SEQ ID NO: 112, 113, or 114. In some embodiments, the second heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 112, 113, or 114. In several embodiments, a third heavy chain CDR has the amino acid sequence of SEQ ID NO: 115. In some embodiments, the third heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 115.

In several embodiments, a first light chain CDR has the amino acid sequence of SEQ ID NO: 108. In some embodiments, the first light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 108. In several embodiments, a second light chain CDR has the amino acid sequence of SEQ ID NO: 109. In some embodiments, the second light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 109. In several embodiments, a third light chain CDR has the amino acid sequence of SEQ ID NO: 110. In some embodiments, the third light chain CDR comprises a sequence of amino acids that is at least 70%, 75%, 80%, 85%, 90%, 91%, 92%, 93%, 94%, 95%, 96%, 97%, 98%, 99% or more, identical to the sequence of SEQ ID NO. 110.

In several embodiments, there is provided an anti-CD19 CAR comprising the amino acid sequence of SEQ ID NO. 116. In some embodiments, there is provided an anti-CD19 CAR comprising a sequence of amino acids that is at least 70%, 75%, 80%, 85%, 90%, 91%, 92%, 93%, 94%, 95%, 96%, 97%, 98%, 99% or more, identical to the sequence of SEQ ID NO. 116.

In one embodiment, there is provided a polynucleotide encoding a Tumor Binder/CD8hinge-CD8TM/OX40/CD3zeta chimeric antigen receptor complex (see FIG. 1, CAR1c). The polynucleotide comprises or is composed of tumor binder, a CD8a hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a tumor binder/CD8hinge-CD8TM/OX40/CD3zeta/2A/m IL-15 chimeric antigen receptor complex (see FIG. 1, CAR 1d). The polynucleotide comprises or is composed of a Tumor Binder, a CD8a hinge, a CD8a transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

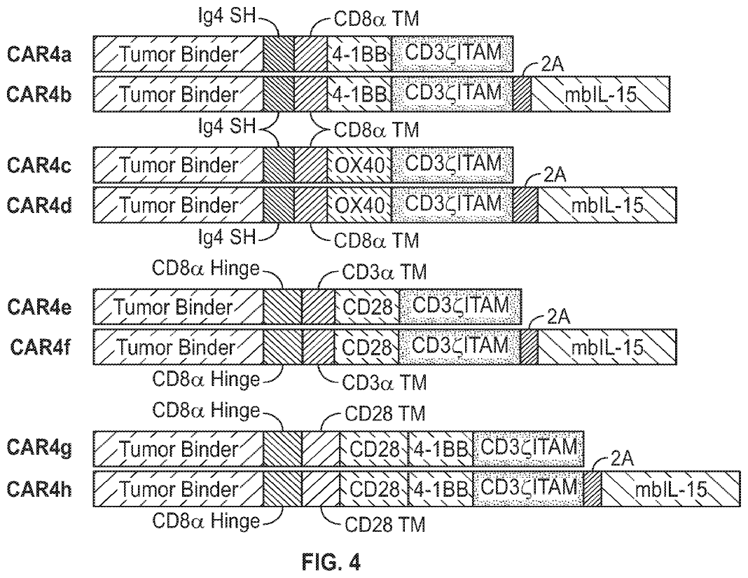

In several embodiments, there is provided a polynucleotide encoding a Tumor Binder/Ig4SH-CD8TM/4-1 BB/CD3zeta chimeric antigen receptor complex (see FIG. 4, CAR4a). The polynucleotide comprises or is composed of a Tumor Binder, an Ig4 SH domain, a CD8a transmembrane domain, a 4-1 BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/4-1BB/CD3zeta/2A/mIL-15 chimeric antigen receptor complex (see FIG. 4, CAR4b). The polynucleotide comprises or is composed of a Tumor Binder, a Ig4 SH domain, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/CD3zeta chimeric antigen receptor complex (see FIG. 1, CAR1e). The polynucleotide comprises or is composed of a Tumor Binder, a CD8a hinge, a CD28 transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/CD3zeta/2A/mIL-15 chimeric antigen receptor complex (see FIG. 1, CAR1f). The polynucleotide comprises or is composed of a Tumor Binder, a CD8a hinge, a CD28 transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

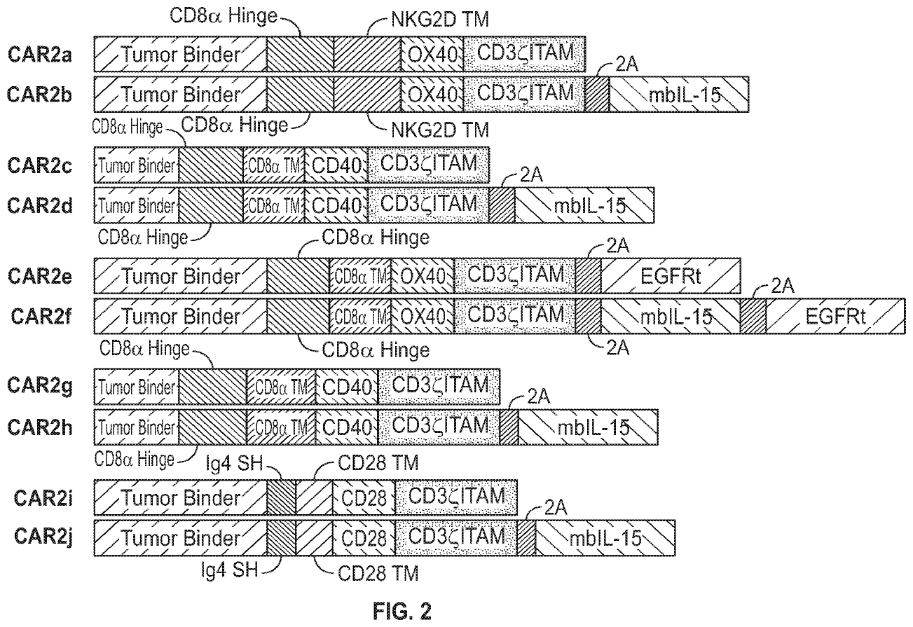

In several embodiments, there is provided a polynucleotide encoding a Tumor Binder/Ig4SH-CD28TM/CD28/CD3zeta chimeric antigen receptor complex (see FIG. 2, CAR2i). The polynucleotide comprises or is composed of a Tumor Binder, an Ig4 SH domain, a CD28 transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD28TM/CD28/CD3zeta/2A/mIL-15 chimeric antigen receptor complex (see FIG. 2, CAR2j). The polynucleotide comprises or is composed of a Tumor Binder, an Ig4 SH domain, a CD28 transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/OX40/CD3zeta chimeric antigen receptor complex (see FIG. 4, CAR4c). The polynucleotide comprises or is composed of a Tumor Binder, a Ig4 SH domain, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/OX40/CD3zeta/2A/mIL-15 chimeric antigen receptor complex (see FIG. 4, CAR4d). The polynucleotide comprises or is composed of a Tumor Binder, a Ig4 SH domain, a CD8a transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD3αTM/CD28/CD3zeta chimeric antigen receptor complex (see FIG. 4, CAR4e). The polynucleotide comprises or is composed of a Tumor Binder, a CD8a hinge, a CD3a transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD3αTM/CD28/CD3zeta/2A/mIL-15 chimeric antigen receptor complex (see FIG. 4, CAR4f). The polynucleotide comprises or is composed of a Tumor Binder, a CD8a hinge, a CD3a transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/4-1 BB/CD3zeta chimeric antigen receptor complex (see FIG. 4, CAR 4g). The polynucleotide comprises or is composed of a Tumor Binder, a CD8a hinge, a CD28 transmembrane domain, a CD28 domain, a 4-1 BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/4-1BB/CD3zeta/2A/mIL-15 chimeric antigen receptor complex (see FIG. 4, CAR 4h). The polynucleotide comprises or is composed of a Tumor Binder, a CD8a hinge, a CD28 transmembrane domain, a CD28 domain, a 4-1 BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

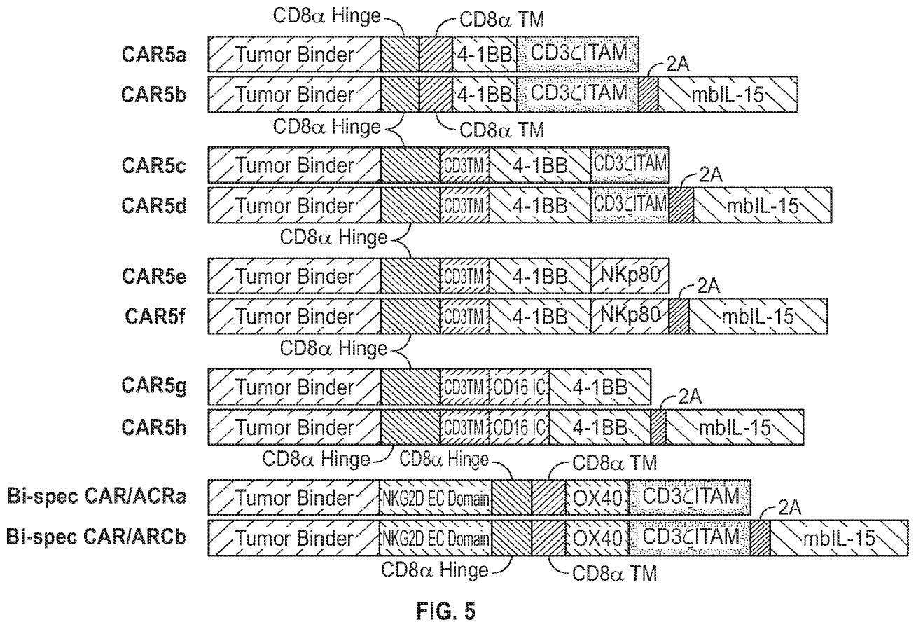

In several embodiments, there is provided a polynucleotide encoding a Tumor Binder/CD8 alpha hinge/CD8 alpha TM/4-1 BB/CD3zeta chimeric antigen receptor complex (see FIG. 5, CAR5a). The polynucleotide comprises or is composed of an anti-CD19 moiety, a CD8a hinge, a CD8a transmembrane domain, a 4-1 BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD8 alpha TM/4-1BB/CD3zeta/2A/mIL-15 chimeric antigen receptor complex (see FIG. 5, CAR 5b). The polynucleotide comprises or is composed of a Tumor Binder, a CD8a hinge, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1 BB/CD3zeta chimeric antigen receptor complex (see FIG. 5, CAR5c). The polynucleotide comprises or is composed of a Tumor Binder, a CD8a hinge, a CD3 transmembrane domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1 BB/CD3zeta/2A/m IL-15 chimeric antigen receptor complex (see FIG. 5, CAR5d). The polynucleotide comprises or is composed of a Tumor Binder, a CD8a hinge, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1BB/NKp80 chimeric antigen receptor complex (see FIG. 5, CAR5e). The polynucleotide comprises or is composed of a Tumor Binder, a CD8a hinge, a CD3 transmembrane domain, a 4-1BB domain, and an NKp80 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1BB/NKp80/2A/mIL-15 chimeric antigen receptor complex (see FIG. 5, CAR5f). The polynucleotide comprises or is composed of a Tumor Binder, a CD8a hinge, a CD8a transmembrane domain, a 4-1BB domain, an NKp80 domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/CD16 intracellular domain/4-1BB chimeric antigen receptor complex (see FIG. 5, CAR5g). The polynucleotide comprises or is composed of a Tumor Binder, a CD8a hinge, a CD3 transmembrane domain, CD16 intracellular domain, and a 4-1BB domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/CD16/4-1BB/2A/mIL-15 chimeric antigen receptor complex (see FIG. 5, CAR5h). The polynucleotide comprises or is composed of a Tumor Binder, a CD8a hinge, a CD8a transmembrane domain, a CD16 intracellular domain, a 4-1BB domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/NKG2D Extracellular Domain/CD8hinge-CD8TM/OX40/CD3zeta chimeric antigen receptor complex (see FIG. 5, Bi-spec CAR/ACRa). The polynucleotide comprises or is composed of a Tumor Binder, an NKG2D extracellular domain (either full length or a fragment), a CD8a hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/NKG2D EC Domain/CD8hinge-CD8TM/OX40/CD3zeta/2A/m IL-15 chimeric antigen receptor complex (see FIG. 5, Bi-spec CAR/ACRb). The polynucleotide comprises or is composed of a Tumor Binder, an NKG2D extracellular domain (either full length or a fragment), a CD8a hinge, a CD8a transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8TM/4-1 BB/CD3zeta chimeric antigen receptor complex (see FIG. 1, CAR1a). The polynucleotide comprises or is composed of a Tumor Binder, a CD8a hinge, a CD8a transmembrane domain, a 4-1 BB domain, and a CD3zeta domain. By way of non-limiting embodiment, there is provided herein an anti-CD19/CD8hinge/CD8TM/4-1BB/CD3zeta chimeric antigen receptor complex. In several embodiments, this receptor complex is encoded by a nucleic acid molecule having the sequence of SEQ ID NO: 85. In several embodiments, a nucleic acid sequence encoding an CAR1a chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 85. In several embodiments, the chimeric receptor comprises the amino acid sequence of SEQ ID NO: 86. In several embodiments, a CAR1a chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 86. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided an CAR1a construct that further comprises mbIL15, as disclosed herein (see e.g., FIG. 1 CAR1b).

In several embodiments, there is provided a polynucleotide encoding a Tumor Binder/CD8hinge/CD8TM/OX40/CD3zeta chimeric antigen receptor complex (see FIG. 1, CAR1c). The polynucleotide comprises or is composed of a Tumor Binder, a CD8a hinge, a CD8a transmembrane domain, an OX40 domain, and a CD3zeta domain. In several embodiments, the chimeric antigen receptor further comprises mbIL15 (see FIG. 1, CAR1d). By way of non-limiting embodiment, there is provided herein an anti CD19/CD8hinge/CD8TM/OX40/CD3zeta/2A/mIL-15 chimeric antigen receptor. In such embodiments, the polynucleotide comprises or is composed of an anti-CD19 scFv, a CD8a hinge, a CD8a transmembrane domain, an OX40 domain, a CD3zeta domain, a 2A cleavage site, and an mbIL-15 domain as described herein. In several embodiments, this receptor complex is encoded by a nucleic acid molecule having the sequence of SEQ ID NO: 59. In several embodiments, a nucleic acid sequence encoding an CAR1d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 59. In several embodiments, the chimeric receptor comprises the amino acid sequence of SEQ ID NO: 60. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 60. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD28TM/CD28/CD3zeta chimeric antigen receptor complex (see FIG. 1, CAR1e). The polynucleotide comprises or is composed of a Tumor Binder, a CD8a hinge, a CD28 transmembrane domain, CD28 signaling domain, and a CD3zeta domain. In several embodiments, the chimeric antigen receptor further comprises mbIL15 (see FIG. 1, CAR1d). By way of non-limiting embodiment, there is provided herein an anti-CD19 moiety/CD8hinge/CD28TM/CD28/CD3zeta/2A/mIL15 chimeric antigen receptor complex. In such embodiments, the polynucleotide comprises or is composed of an anti-CD19 scFv, a CD8a hinge, a CD28 transmembrane domain, CD28 signaling domain, a CD3zeta domain a 2A cleavage site, and an mbIL-15 domain as described herein. In several embodiments, this receptor complex is encoded by a nucleic acid molecule having the sequence of SEQ ID NO: 61. In several embodiments, a nucleic acid sequence encoding an CAR1d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 61. In several embodiments, the chimeric receptor comprises the amino acid sequence of SEQ ID NO: 62. In several embodiments, a CAR1d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 62. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/ICOS/CD3zeta chimeric antigen receptor complex (see FIG. 1, CAR1g). The polynucleotide comprises or is composed of a Tumor Binder, a CD8a hinge, a CD8a transmembrane domain, inducible costimulator (ICOS) signaling domain, and a CD3zeta domain. In several embodiments, the chimeric antigen receptor further comprises mbIL15 (see 1, CAR1h). By way of non-limiting embodiment, there is provided herein an anti-CD19moiety/CD8hinge/CD8aTM/ICOS/CD3zeta/2A/mIL15 chimeric antigen receptor complex. In such embodiments, the polynucleotide comprises or is composed of an anti-CD19 scFv, a CD8a hinge, a CD8a transmembrane domain, inducible costimulator (ICOS) signaling domain, a CD3zeta domain, a 2A cleavage site, and an mbIL-15 domain as described herein. In several embodiments, this receptor complex is encoded by a nucleic acid molecule having the sequence of SEQ ID NO: 63. In several embodiments, a nucleic acid sequence encoding an CAR1 h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 63. In several embodiments, the chimeric receptor comprises the amino acid sequence of SEQ ID NO: 64. In several embodiments, a CAR1 h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 64. In several embodiments, the CAR1 h scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

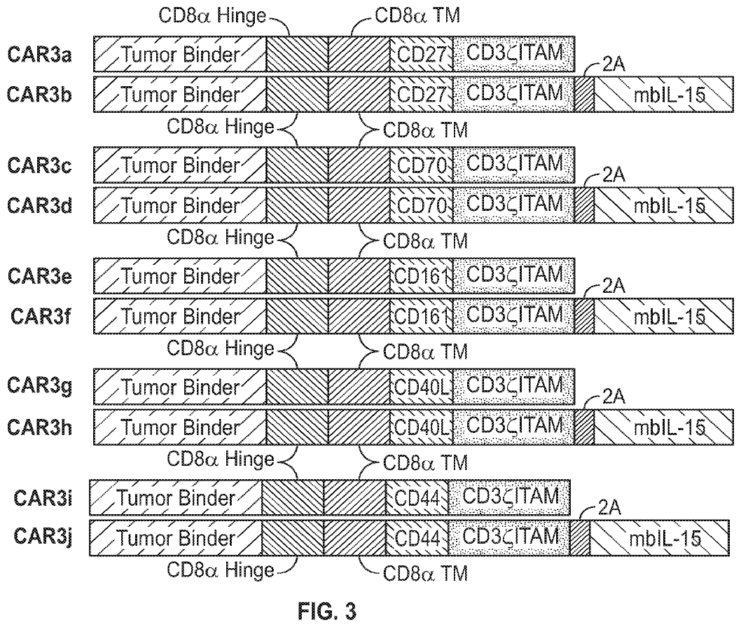

In several embodiments, there is provided a polynucleotide encoding a Tumor Binder/CD8hinge/CD8aTM/CD28/4-1 BB/CD3zeta chimeric antigen receptor complex (see FIG. 1, CAR1i). The polynucleotide comprises or is composed of a Tumor Binder, a CD8a hinge, a CD8a transmembrane domain, a CD28 signaling domain, a 4-1BB signaling domain, and a CD3zeta domain. In several embodiments, the chimeric antigen receptor further comprises mbIL15 (see FIG. 3A, NK19-4b). By way of non-limiting embodiment, there is provided herein an anti-CD19moiety/CD8hinge/CD8aTM/CD28/4-1BB/CD3zeta/2A/mIL-15. In such embodiments, the polynucleotide comprises or is composed of an anti-CD19 scFv, a CD8a hinge, a CD8a transmembrane domain, a CD28 signaling domain, a 4-1 BB signaling domain, a CD3zeta domain, a 2A cleavage site, and an mbIL-15 domain as described herein. In several embodiments, this receptor complex is encoded by a nucleic acid molecule having the sequence of SEQ ID NO: 65. In several embodiments, a nucleic acid sequence encoding an CAR1 h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 65. In several embodiments, the chimeric receptor comprises the amino acid sequence of SEQ ID NO: 66. In several embodiments, a CAR1 h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 66. In several embodiments, the CAR1 h scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/NKG2DTM/OX40/CD3zeta chimeric antigen receptor complex (see FIG. 2, CAR2a). The polynucleotide comprises or is composed of a Tumor Binder, a CD8a hinge, a NKG2D transmembrane domain, an OX40 signaling domain, and a CD3zeta domain. In several embodiments, the chimeric antigen receptor further comprises mbIL15 (see FIG. 2, CAR2b). By way of non-limiting embodiment, there is provided herein an anti-CD19moiety/CD8hinge/NKG2DTM/OX40/CD3zeta/2A/mIL-15 chimeric antigen receptor complex. In such embodiments, the polynucleotide comprises or is composed of an anti-CD19 scFv, a CD8a hinge, a NKG2D transmembrane domain, an OX40 signaling domain, a CD3zeta domain, a 2A cleavage site, and an mbIL-15 domain as described herein. In several embodiments, this receptor complex is encoded by a nucleic acid molecule having the sequence of SEQ ID NO: 67. In several embodiments, a nucleic acid sequence encoding an CAR2b chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 67. In several embodiments, the chimeric receptor comprises the amino acid sequence of SEQ ID NO: 68. In several embodiments, a CAR2b chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 68. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40/CD3zeta chimeric antigen receptor complex (see FIG. CAR2c). The polynucleotide comprises or is composed of Tumor Binder, a CD8a hinge, a CD8a transmembrane domain, a CD40 signaling domain, and a CD3zeta domain. In several embodiments, the chimeric antigen receptor further comprises mbIL15 (see FIG. 1, CAR2d). By way of non-limiting embodiment, there is provided herein an anti-CD19moiety/CD8hinge/CD8aTM/CD40/CD3zeta/2A/mIL-15 chimeric antigen receptor complex. In such embodiments, the polynucleotide comprises or is composed of an anti-CD19 scFv variable heavy chain, a CD8a hinge, a CD8a transmembrane domain, a CD40 signaling domain, a CD3zeta domain, a 2A cleavage site, and an mbIL-15 domain as described herein. In several embodiments, this receptor complex is encoded by a nucleic acid molecule having the sequence of SEQ ID NO: 69. In several embodiments, a nucleic acid sequence encoding an CAR2d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 69. In several embodiments, the chimeric receptor comprises the amino acid sequence of SEQ ID NO: 70. In several embodiments, a CAR2d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 70. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/OX40/CD3zeta/2A/EGFRt chimeric antigen receptor complex (see FIG. 2, CAR2e). The polynucleotide comprises or is composed of a Tumor Binder, a CD8a hinge, a CD8a transmembrane domain, an OX40 signaling domain, a CD3zeta domain, a 2A cleavage side, and a truncated version of the epidermal growth factor receptor (EGFRt). In several embodiments, the chimeric antigen receptor further comprises mbIL15 (see FIG. 2, CAR2f). By way of non-limiting embodiment, there is provided herein an anti-CD19moiety/CD8hinge/CD8aTM/OX40/CD3zeta/2A/mIL-15/2A/EGFRt chimeric antigen receptor complex. In such embodiments, the polynucleotide comprises or is composed of an anti-CD19 scFv, a CD8a hinge, a CD8a transmembrane domain, an OX40 signaling domain, a CD3zeta domain, a 2A cleavage side, a truncated version of the epidermal growth factor receptor (EGFRt), an additional 2A cleavage site, and an mbIL-15 domain as described herein. In several embodiments, this receptor complex is encoded by a nucleic acid molecule having the sequence of SEQ ID NO: 71. In several embodiments, a nucleic acid sequence encoding an CAR2f chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 71. In several embodiments, the chimeric receptor comprises the amino acid sequence of SEQ ID NO: 72. In several embodiments, a CAR2f chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 72. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40/CD3zeta chimeric antigen receptor complex (see FIG. 2, CAR2g). The polynucleotide comprises or is composed of a Tumor Binder, a CD8a hinge, a CD8a transmembrane domain, a CD40 signaling domain, and a CD3zeta domain. In several embodiments, the chimeric antigen receptor further comprises mbIL15 (see FIG. 2, CAR2h). By way of non-limiting embodiment, there is provided herein an anti-CD19moiety/CD8hinge/CD8aTM/CD40/CD3zeta/2A/mIL-15 chimeric antigen receptor complex. In such embodiments, the polynucleotide comprises or is composed of an anti-CD19 scFv variable light chain, a CD8a hinge, a CD8a transmembrane domain, a CD40 signaling domain, a CD3zeta domain, a 2A cleavage site, and an mbIL-15 domain as described herein. In several embodiments, this receptor complex is encoded by a nucleic acid molecule having the sequence of SEQ ID NO: 73. In several embodiments, a nucleic acid sequence encoding an CAR2h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 73. In several embodiments, the chimeric receptor comprises the amino acid sequence of SEQ ID NO: 74. In several embodiments, a CAR2h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 74. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD27/CD3zeta chimeric antigen receptor complex (see FIG. 3, CAR3a). The polynucleotide comprises or is composed of a Tumor Binder, a CD8a hinge, a CD8a transmembrane domain, a CD27 signaling domain, and a CD3zeta domain. In several embodiments, the chimeric antigen receptor further comprises mbIL15 (see FIG. 3, CAR3b). By way of non-limiting embodiment, there is provided herein an anti-CD19moiety/CD8hinge/CD8aTM/CD27/CD3zeta/2A/mIL-15 chimeric antigen receptor complex. In such embodiments, the polynucleotide comprises or is composed of an anti-CD19 scFv, a CD8a hinge, a CD8a transmembrane domain, a CD27 signaling domain, a CD3zeta domain, a 2A cleavage site, and an mbIL-15 domain as described herein. In several embodiments, this receptor complex is encoded by a nucleic acid molecule having the sequence of SEQ ID NO: 75. In several embodiments, a nucleic acid sequence encoding an CAR3b chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 75. In several embodiments, the chimeric receptor comprises the amino acid sequence of SEQ ID NO: 76. In several embodiments, a CAR3b chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 76. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD70/CD3zeta chimeric antigen receptor complex (see FIG. 3, CAR3c). The polynucleotide comprises or is composed of a Tumor Binder, a CD8a hinge, a CD8a transmembrane domain, a CD70 signaling domain, and a CD3zeta domain. In several embodiments, the chimeric antigen receptor further comprises mbIL15 (see FIG. 3, CAR3d). By way of non-limiting embodiment, there is provided herein an anti-CD19moiety/CD8hinge/CD8aTM/CD70/CD3zeta/2A/mIL-15 chimeric antigen receptor complex. In such embodiments, the polynucleotide comprises or is composed of an anti-CD19 scFv, a CD8a hinge, a CD8a transmembrane domain, a CD70 signaling domain, a CD3zeta domain, a 2A cleavage site, and an mbIL-15 domain as described herein. In several embodiments, this receptor complex is encoded by a nucleic acid molecule having the sequence of SEQ ID NO: 77. In several embodiments, a nucleic acid sequence encoding an CAR3d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 77. In several embodiments, the chimeric receptor comprises the amino acid sequence of SEQ ID NO: 78. In several embodiments, a CAR3d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 78. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD161/CD3zeta chimeric antigen receptor complex (see FIG. 3, CAR3e). The polynucleotide comprises or is composed of a Tumor Binder, a CD8a hinge, a CD8a transmembrane domain, a CD161 signaling domain, and a CD3zeta domain. In several embodiments, the chimeric antigen receptor further comprises mbIL15 (see FIG. 3, CAR3f). By way of non-limiting embodiment, there is provided herein an anti-CD19moiety/CD8hinge/CD8aTM/CD161/CD3zeta/2A/mIL-15 chimeric antigen receptor complex. In such embodiments, the polynucleotide comprises or is composed of an anti-CD19 scFv, a CD8a hinge, a CD8a transmembrane domain, a CD161 signaling domain, a CD3zeta domain, a 2A cleavage site, and an mbIL-15 domain as described herein. In several embodiments, this receptor complex is encoded by a nucleic acid molecule having the sequence of SEQ ID NO: 79. In several embodiments, a nucleic acid sequence encoding an CAR3f chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 79. In several embodiments, the chimeric receptor comprises the amino acid sequence of SEQ ID NO: 80. In several embodiments, a CAR3f chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 80. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40L/CD3zeta chimeric antigen receptor complex (see FIG. 3, CAR3g). The polynucleotide comprises or is composed of a Tumor Binder, a CD8a hinge, a CD8a transmembrane domain, a CD40L signaling domain, and a CD3zeta domain. In several embodiments, the chimeric antigen receptor further comprises mbIL15 (see FIG. 3, CAR3h). By way of non-limiting embodiment, there is provided herein an anti-CD19moiety/CD8hinge/CD8aTM/CD40L/CD3zeta/2A/mIL-15 chimeric antigen receptor complex. In such embodiments, the polynucleotide comprises or is composed of an anti-CD19 scFv, a CD8a hinge, a CD8a transmembrane domain, a CD40L signaling domain, a CD3zeta domain, a 2A cleavage site, and an mbIL-15 domain as described herein. In several embodiments, this receptor complex is encoded by a nucleic acid molecule having the sequence of SEQ ID NO: 81. In several embodiments, a nucleic acid sequence encoding an CAR3h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 81. In several embodiments, the chimeric receptor comprises the amino acid sequence of SEQ ID NO: 82. In several embodiments, a CAR3h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 82. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD44/CD3zeta chimeric antigen receptor complex (see FIG. 3, CAR3i). The polynucleotide comprises or is composed of a Tumor Binder, a CD8a hinge, a CD8a transmembrane domain, a CD44 signaling domain, and a CD3zeta domain. In several embodiments, the chimeric antigen receptor further comprises mbIL15 (see FIG. 3, CAR3j). By way of non-limiting embodiment, there is provided herein an anti-CD19moiety/CD8hinge/CD8aTM/CD44/CD3zeta/2A/mIL-15 chimeric antigen receptor complex. In such embodiments, the polynucleotide comprises or is composed of an anti-CD19 scFv, a CD8a hinge, a CD8a transmembrane domain, a CD44 signaling domain, a CD3zeta domain, a 2A cleavage site, and an mbIL-15 domain as described herein. In several embodiments, this receptor complex is encoded by a nucleic acid molecule having the sequence of SEQ ID NO: 83. In several embodiments, a nucleic acid sequence encoding an CAR3j chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 83. In several embodiments, the chimeric receptor comprises the amino acid sequence of SEQ ID NO: 84. In several embodiments, a CAR3j chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 84. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding an anti CD123/CD8a hinge/CD8a transmembrane domain/OX40/CD3zeta chimeric antigen receptor complex (see FIG. 6, CD123 CARa). The polynucleotide comprises or is composed of an anti CD123 moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided an CD123 CAR construct that further comprises mbIL15, as disclosed herein (see e.g., FIG. 6, CD123 CARb).

In several embodiments, there is provided a polynucleotide encoding an anti CLDN6/CD8a hinge/CD8a transmembrane domain/OX40/CD3zeta chimeric antigen receptor complex (see FIG. 6, CLDN6 CARa). The polynucleotide comprises or is composed of an anti CLDN6 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a CLDN6 CAR construct that further comprises mbIL15, as disclosed herein (see e.g., FIG. 6, CLDN6 CARb).

Depending on the embodiment, various binders can be used to target CLDN6. In several embodiments, peptide binders are used, while in some embodiments antibodies, or fragments thereof are used. In several embodiments employing antibodies, antibody sequences are optimized, humanized or otherwise manipulated or mutated from their native form in order to increase one or more of stability, affinity, avidity or other characteristic of the antibody or fragment. In several embodiments, an antibody is provided that is specific for CLDN6. In several embodiments, an scFv is provided that is specific for CLDN6. In several embodiments, the antibody or scFv specific for CLDN6 comprises a heavy chain variable comprising the amino acid sequence of SEQ ID NO. 88. In some embodiments, the heavy chain variable comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 88. In some embodiments, the heavy chain variable comprises a sequence of amino acids that is encoded by a polynucleotide that hybridizes under moderately stringent conditions to the complement of a polynucleotide that encodes a heavy chain variable of SEQ ID NO. 88. In some embodiments, the heavy chain variable domain a sequence of amino acids that is encoded by a polynucleotide that hybridizes under stringent conditions to the complement of a polynucleotide that encodes a heavy chain variable encodes a heavy chain variable of SEQ ID NO. 88.

In several embodiments, the antibody or scFv specific for CLDN6 comprises a light chain variable comprising the amino acid sequence of any of SEQ ID NO. 89, 90, or 91. In several embodiments, the light chain variable comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the identical to the sequence of SEQ ID NO. 89, 90, or 91. In some embodiments, the light chain variable comprises a sequence of amino acids that is encoded by a polynucleotide that hybridizes under moderately stringent conditions to the complement of a polynucleotide that encodes a light chain variable of SEQ ID NO. 89, 90, or 91. In some embodiments, the light chain variable domain comprises a sequence of amino acids that is encoded by a polynucleotide that hybridizes under stringent conditions to the complement of a polynucleotide that encodes a light chain variable domain of SEQ ID NO. 89, 90, or 91.

In several embodiments, the anti-CLDN6 antibody or scFv comprises one, two, or three heavy chain complementarity determining region (CDR) and one, two, or three light chain CDRs. In several embodiments, a first heavy chain CDR has the amino acid sequence of SEQ ID NO: 92. In some embodiments, the first heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 92. In several embodiments, a second heavy chain CDR has the amino acid sequence of SEQ ID NO: 93. In some embodiments, the second heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 93. In several embodiments, a third heavy chain CDR has the amino acid sequence of SEQ ID NO: 94. In some embodiments, the third heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 94.

In several embodiments, a first light chain CDR has the amino acid sequence of SEQ ID NO: 95, 98, or 101. In some embodiments, the first light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 95, 98, or 101. In several embodiments, a second light chain CDR has the amino acid sequence of SEQ ID NO: 96, 99, or 102. In some embodiments, the second light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 96, 99, or 102. In several embodiments, a third light chain CDR has the amino acid sequence of SEQ ID NO: 97, 100, or 103. In some embodiments, the third light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 97, 100, or 103.

Advantageously, in several embodiments, the CLDN6 CARs are highly specific to CLDN6 and do not substantially bind to any of CLDN3, 4, or 9.

In several embodiments, there is provided a polynucleotide encoding an anti BCMA/CD8a hinge/CD8a transmembrane domain/OX40/CD3zeta chimeric antigen receptor complex (see FIG. 6, BCMA CARa). The polynucleotide comprises or is composed of an anti BCMA binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a BCMA CAR construct that further comprises mbIL15, as disclosed herein (see e.g., FIG. 6, BCMA CARb).

In several embodiments, there is provided a polynucleotide encoding an anti HER2/CD8a hinge/CD8a transmembrane domain/OX40/CD3zeta chimeric antigen receptor complex (see FIG. 6, HER2 CARa). The polynucleotide comprises or is composed of an anti HER2 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a HER2 CAR construct that further comprises mbIL15, as disclosed herein (see e.g., FIG. 6, HER2 CARb).

In several embodiments, there is provided a polynucleotide encoding an NKG2D/CD8a hinge/CD8a transmembrane domain/OX40/CD3zeta activating chimeric receptor complex (see FIG. 6, NKG2D ACRa). The polynucleotide comprises or is composed of a fragment of the NKG2D receptor capable of binding a ligand of the NKG2D receptor, a CD8alpha hinge, a CD8a transmembrane domain, an OX40 domain, and a CD3zeta domain as described herein. In several embodiments, this receptor complex is encoded by a nucleic acid molecule comprising the nucleic acid sequence of SEQ ID NO: 145. In yet another embodiment, this chimeric receptor is encoded by the amino acid sequence of SEQ ID NO: 174. In some embodiments, the sequence of the chimeric receptor may vary from SEQ ID NO. 145, but remains, depending on the embodiment, at least 70%, at least 75%, at least 80%, at least 85%, at least 90%, or at least 95% homologous with SEQ ID NO. 145. In several embodiments, while the chimeric receptor may vary from SEQ ID NO. 145, the chimeric receptor retains, or in some embodiments, has enhanced, NK cell activating and/or cytotoxic function. Additionally, in several embodiments, this construct can optionally be co-expressed with mbIL15 (FIG. 7, NKG2D ACRb). Additional information about chimeric receptors for use in the presently disclosed methods and compositions can be found in PCT Patent Publication No. WO/2018/183385, which is incorporated in its entirety by reference herein.

In several embodiments, there is provided a polynucleotide encoding an anti CD70/CD8a hinge/CD8a transmembrane domain/OX40/CD3zeta chimeric antigen receptor complex (see FIG. 7, CD70 CARa). The polynucleotide comprises or is composed of an anti CD70 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a CD70 CAR construct that further comprises mbIL15, as disclosed herein (see e.g., FIG. 7, CD70 CARb).

In several embodiments, there is provided a polynucleotide encoding an anti mesothelin/CD8a hinge/CD8a transmembrane domain/OX40/CD3zeta chimeric antigen receptor complex (see FIG. 7, Mesothelin CARa). The polynucleotide comprises or is composed of an anti mesothelin binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a Mesothelin CAR construct that further comprises mbIL15, as disclosed herein (see e.g., FIG. 7, Mesothelin CARb).

In several embodiments, there is provided a polynucleotide encoding an anti PD-L1/CD8a hinge/CD8a transmembrane domain/OX40/CD3zeta chimeric antigen receptor complex (see FIG. 7, PD-L1 CARa). The polynucleotide comprises or is composed of an anti PD-L1 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a PD-L1 CAR construct that further comprises mbIL15, as disclosed herein (see e.g., FIG. 7, PD-L1 CARb).

In several embodiments, there is provided a polynucleotide encoding an anti EGFR/CD8a hinge/CD8a transmembrane domain/OX40/CD3zeta chimeric antigen receptor complex (see FIG. 7, EGFR CARa). The polynucleotide comprises or is composed of an anti EGFR binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a EGFR CAR construct that further comprises mbIL15, as disclosed herein (see e.g., FIG. 7, EGFR CARb).

In several embodiments, an expression vector, such as a MSCV-IRES-GFP plasmid, a non-limiting example of which is provided in SEQ ID NO: 87, is used to express any of the chimeric antigen receptors provided for herein.

### Methods of Treatment

Some embodiments relate to a method of treating, ameliorating, inhibiting, or preventing cancer with a cell or immune cell comprising a chimeric antigen receptor and/or an activating chimeric receptor, as disclosed herein. In some embodiments, the method includes treating or preventing cancer. In some embodiments, the method includes administering a therapeutically effective amount of immune cells expressing a tumor-directed chimeric antigen receptor and/or tumor-directed chimeric receptor as described herein. Examples of types of cancer that may be treated as such are described herein.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Advantageously, the non-alloreactive engineered T cells disclosed herein further enhance one or more of the above. Administration can be by a variety of routes, including, without limitation, intravenous, intra-arterial, subcutaneous, intramuscular, intrahepatic, intraperitoneal and/or local delivery to an affected tissue.

### Administration and Dosing

Further provided herein are methods of treating a subject having cancer, comprising administering to the subject a composition comprising immune cells (such as NK and/or T cells) engineered to express a cytotoxic receptor complex as disclosed herein. For example, some embodiments of the compositions and methods described herein relate to use of a tumor-directed chimeric antigen receptor and/or tumor-directed chimeric receptor, or use of cells expressing a tumor-directed chimeric antigen receptor and/or tumor-directed chimeric receptor, for treating a cancer patient. Uses of such engineered immune cells for treating cancer are also provided.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Each of these comparisons are versus, for example, a different therapy for a disease, which includes a cell-based immunotherapy for a disease using cells that do not express the constructs disclosed herein. Advantageously, the non-alloreactive engineered T cells disclosed herein further enhance one or more of the above.

Administration can be by a variety of routes, including, without limitation, intravenous, intra-arterial, subcutaneous, intramuscular, intrahepatic, intraperitoneal and/or local delivery to an affected tissue. Doses of immune cells such as NK and/or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of immune cells such as NK and/or T cells is administered, for example between about 1×10⁶ cells/kg to about 1×10⁸ cells/kg. Depending on the embodiment, various types of cancer can be treated. In several embodiments, hepatocellular carcinoma is treated. Additional embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, glioblastoma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-174 (or combinations of two or more of SEQ ID NOS: 1-174) and that also exhibit one or more of the functions as compared with the respective SEQ ID NOS. 1-174 (or combinations of two or more of SEQ ID NOS: 1-174) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, polynucleotides encoding the disclosed cytotoxic receptor complexes are mRNA. In some embodiments, the polynucleotide is DNA. In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a retrovirus.

Further provided herein are engineered immune cells (such as NK and/or T cells) comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein. Additionally, there are provided herein compositions comprising a mixture of engineered immune cells (such as NK cells and/or engineered T cells), each population comprising the polynucleotide, vector, or cytotoxic receptor complexes as disclosed herein and the T cell population having been genetically modified to reduce/eliminate gvHD and/or HvD. In some embodiments, the NK cells and the T cells are from the same donor. In some embodiments, the NK cells and the T cells are from different donors.

Doses of immune cells such as NK cells or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of NK cells is administered, for example between about 1×10⁶ cells/kg to about 1×10⁸ cells/kg. Depending on the embodiment, various types of cancer or infection disease can be treated.

### Cancer Types

Some embodiments of the compositions and methods described herein relate to administering immune cells comprising a tumor-directed chimeric antigen receptor and/or tumor-directed chimeric receptor to a subject with cancer. Various embodiments provided for herein include treatment or prevention of the following non-limiting examples of cancers. Examples of cancer include, but are not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

### Cancer Targets

Some embodiments of the compositions and methods described herein relate to immune cells comprising a chimeric receptor that targets a cancer antigen. Non-limiting examples of target antigens include: CD5, CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(I-4)bDGlcp(I-I)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gpIOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(I-4)bDGlcp(I-I)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-I or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLI), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GMI, PTK7, gpNMB, CDH1-CD324, DLL3, CD276/B7H3, ILI IRa, IL13Ra2, CD179b-IGLII, TCRgamma-delta, NKG2D, CD32 (FCGR2A), Tn ag, TimI−/HVCR1, CSF2RA (GM-CSFR-alpha), TGFbetaR2, Lews Ag, TCR-betal chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLVI-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, ClaudinI 8.2 (CLD18A2 or CLDN18A.2)), P-glycoprotein, STEAP1, LivI, Nectin-4, Cripto, gpA33, BST1/CD157, low conductance chloride channel, and the antigen recognized by TNT antibody.

### Examples

The following are non-limiting descriptions of experimental methods and materials that were used in examples disclosed below.

To further build on various embodiments disclosed herein, several genes that mediate NK function through different pathways were selected in order to evaluate the impact of reducing/eliminating their expression through gene editing techniques. These initial targets represent non-limiting examples of the type of gene that can be edited according to embodiments disclosed herein to enhance one or more aspect of immune cell-mediated immunotherapy, whether utilizing engineered NK cells, engineered T cells, or combinations thereof. The tumor microenvironment (TME), as suggested with the nomenclature, is the environment around a tumor, which includes the surrounding blood vessels and capillaries, immune cells circulating through or retained in the area, fibroblasts, various signaling molecules related by the tumor cells, the immune cells or other cells in the area, as well as the surrounding extracellular matrix. Various mechanisms are employed by tumors to evade detection and/or destruction by host immune cells, including modification of the TME. Tumors may alter the TME by releasing extracellular signals, promoting tumor angiogenesis or even inducing immune tolerance, in part by limiting immune cell entry in the TME and/or limiting reproduction/expansion of immune cells in the TME. The tumor can also modify the ECM, which can allow pathways to develop for tumor extravasation to new sites. Transforming Growth-Factor beta (TGFb) has beneficial effects when reducing inflammation and preventing autoimmunity. However, it can also function to inhibit anti-tumor immune responses, and thus, upregulated expression of TGFb has been implicated in tumor progression and metastasis. TGFb signaling can inhibit the cytotoxic function of NK cells by interacting with the TGFb receptor expressed by NK cells, for example the TGFb receptor isoform II (TGFBR2). In accordance with several embodiments disclosed herein, the reduction or elimination of expression of TGFBR2 through gene editing (e.g., by CRISPr/Cas9 guided by a TGFBR2 guide RNA) interrupts the inhibitory effect of TGFb on NK cells.

As discussed above, the CRISPR/Cas9 system was used to specifically target and reduce the expression of the TGFBR2 by NK cells. Various non-limiting examples of guide RNAs were tested, which are summarized below.

TABLE 1
TGFb Receptor Type 2 Isoform Guide RNAs

| SEQ | | | |
|---|---|---|---|
| ID NO: | Name | Sequence | Target |
| 147 | TGFBR2-1 | CCCCTACCATGACTTTATTC | Exon 4 |
| 148 | TGFBR2-2 | ATTGCACTCATCAGAGCTAC | Exon 4 |
| 149 | TGFBR2-3 | AGTCATGGTAGGGGAGCTTG | Exon 4 |
| 150 | TGFBR2-4 | TGCTGGCGATACGCGTCCAC | Exon 1 |
| 151 | TGFBR2-5 | GTGAGCAATCCCCCGGGCGA | Exon 4 |
| 152 | TGFBR2-6 | AACGTGCGGTGGGATCGTGC | Exon 1 |

Briefly, cryopreserved purified NK cells were thawed on Day 0 and subject to electroporation with CRISPr/Cas9 and a single (or two) guide RNA (using established commercially available transfection guidelines) and were then subsequently cultured in 400 IU/ml IL-2 media for 1 day, followed by 40 IU/ml IL-2 culture with feeder cells (e.g., modified K562 cells expressing, for example, 4-1 BBL and/or mbIL15). At Day 7, knockout efficiency was determined and NK cells were transduced with a virus encoding the NK19-1 CAR construct (as a non-limiting example of a CAR). At Day 14, the knockout efficiency was determined by flow cytometry or other means and cytotoxicity of the resultant NK cells was evaluated.

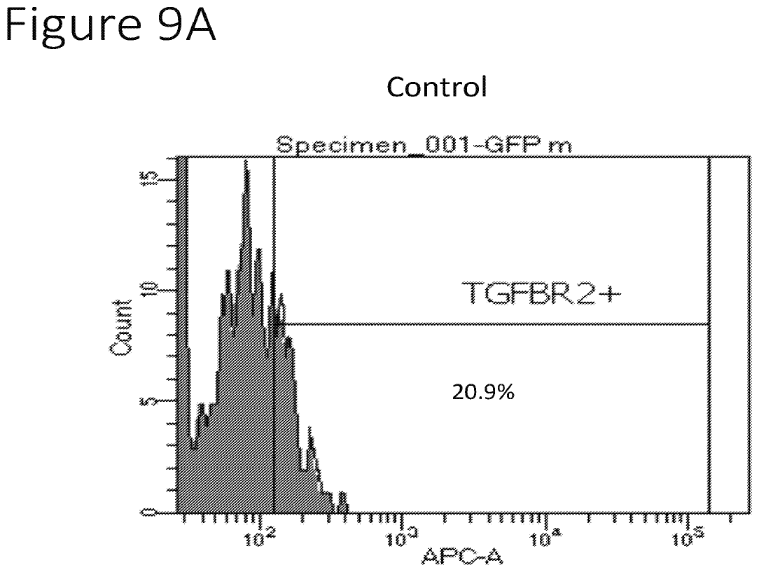

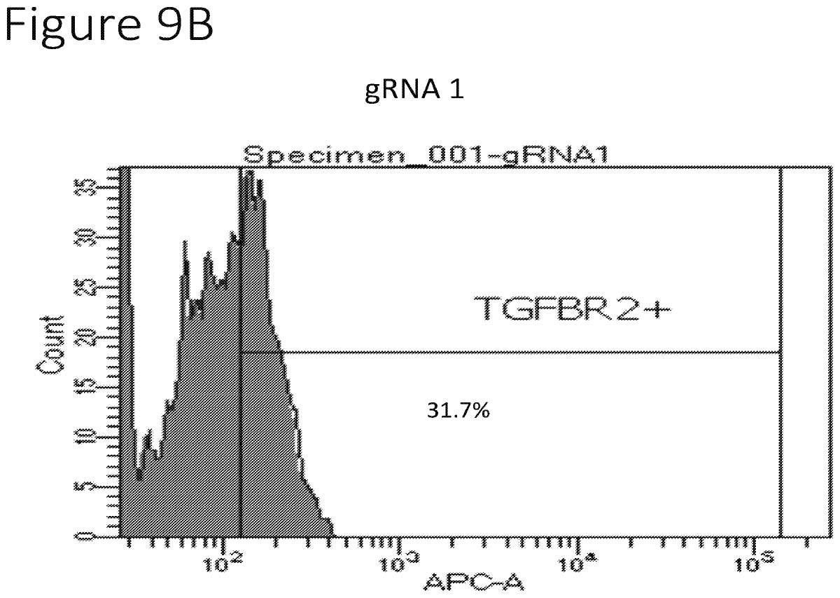

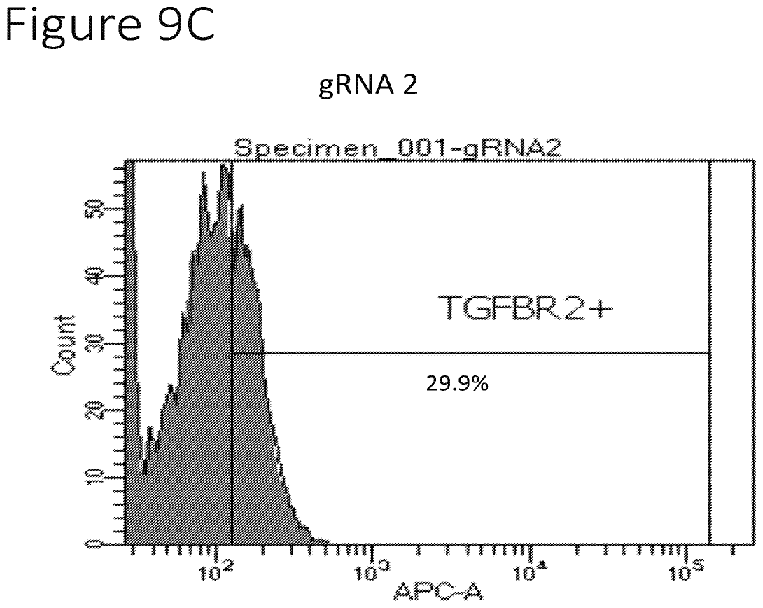

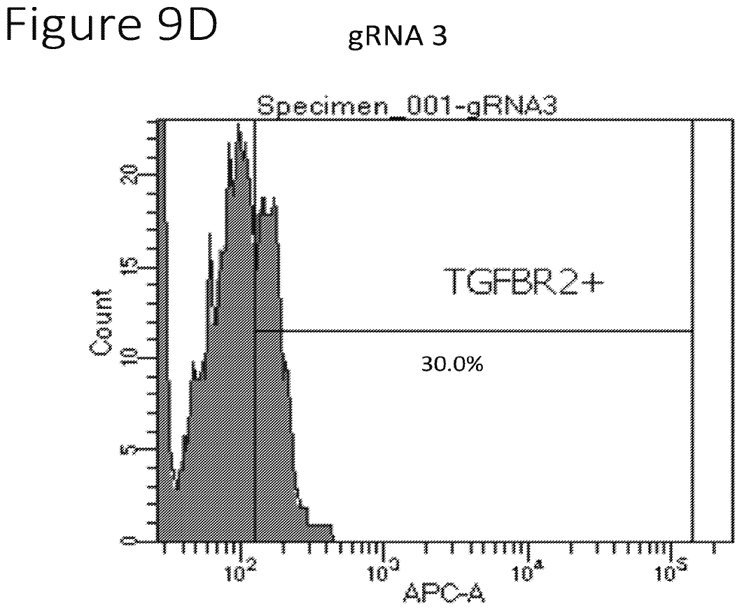

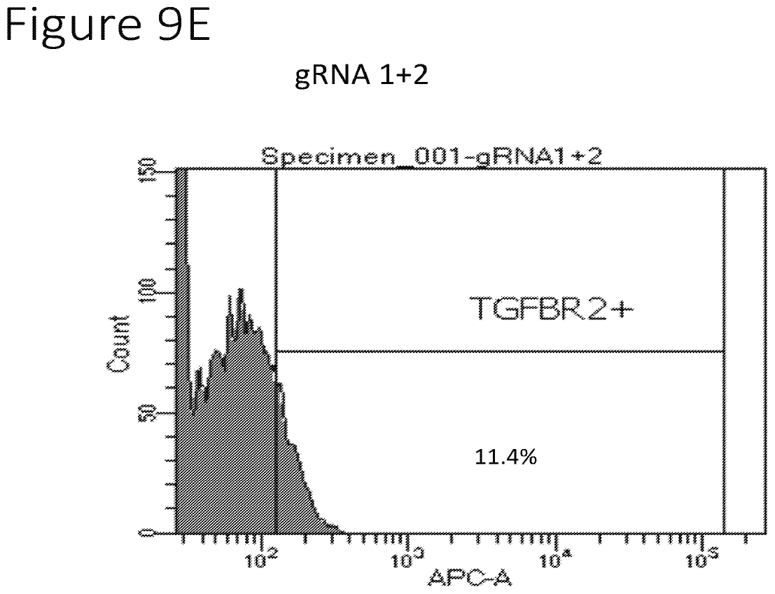

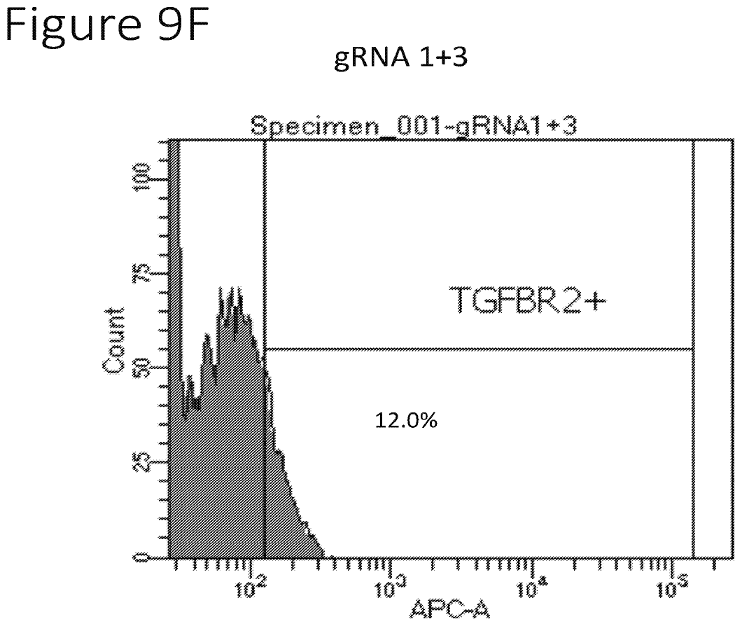

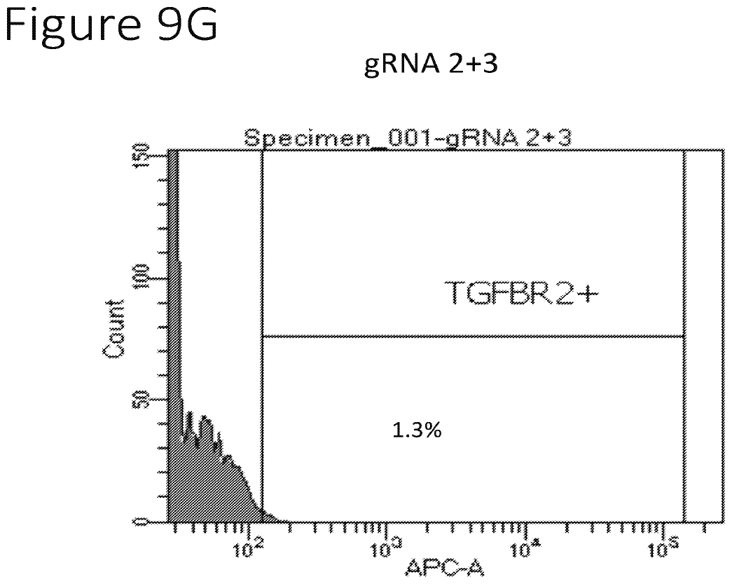

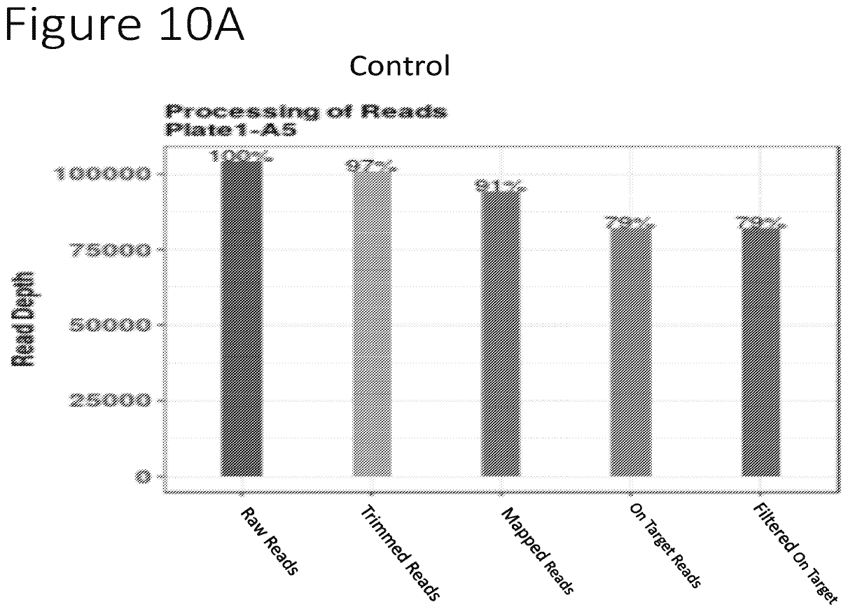

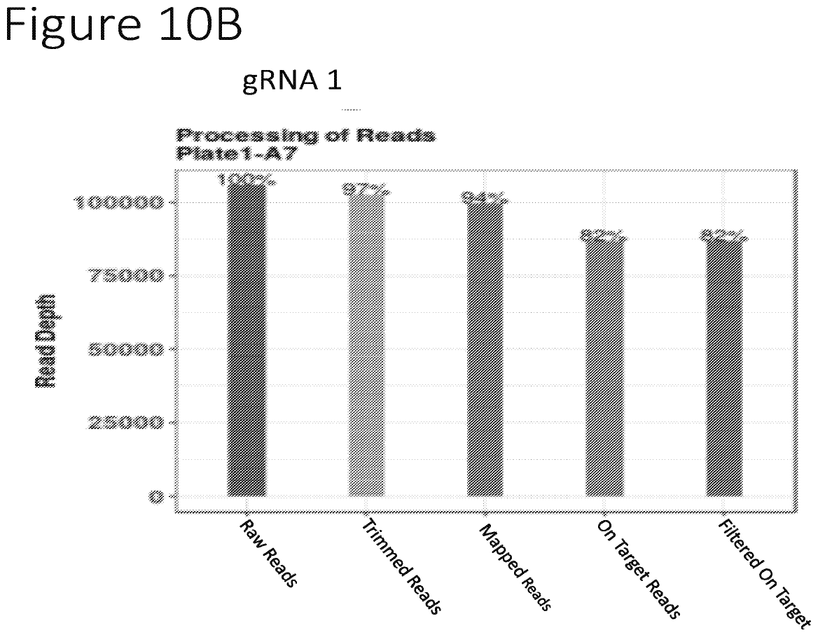

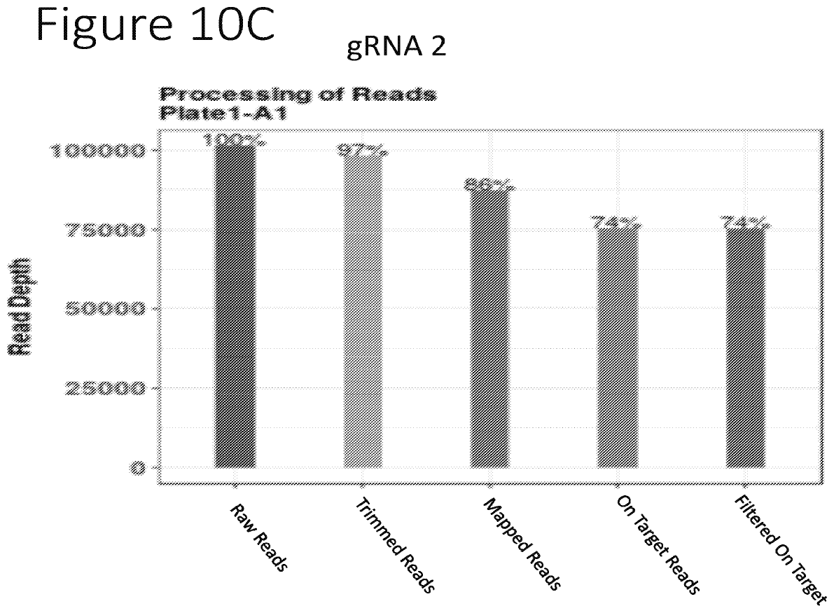

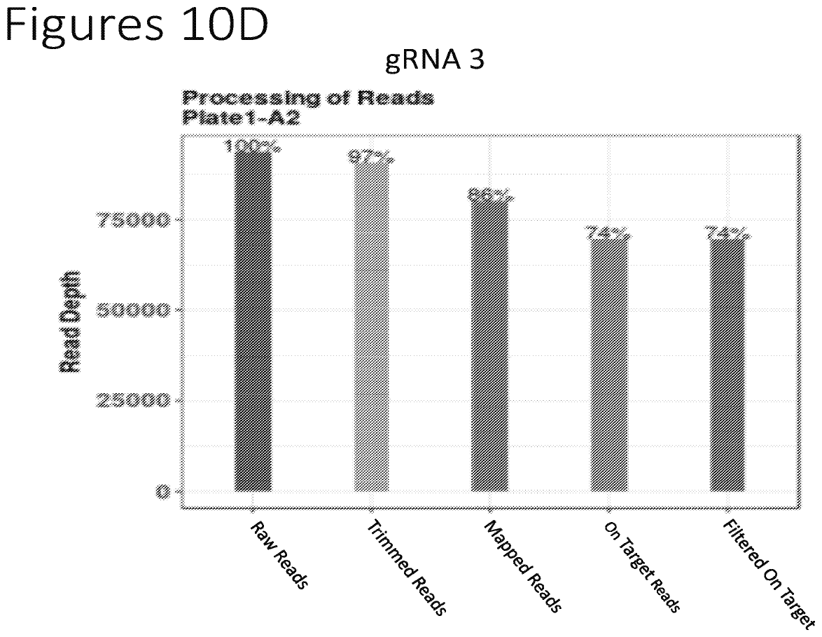

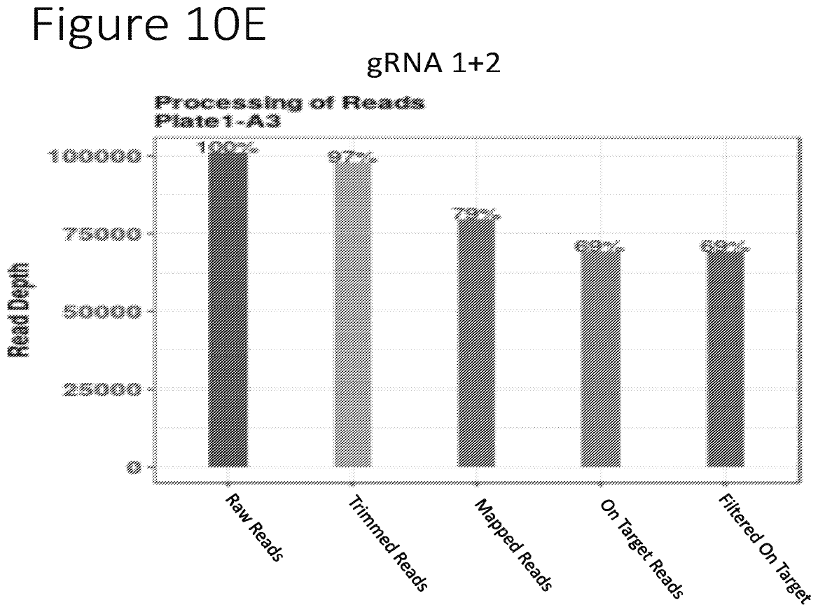

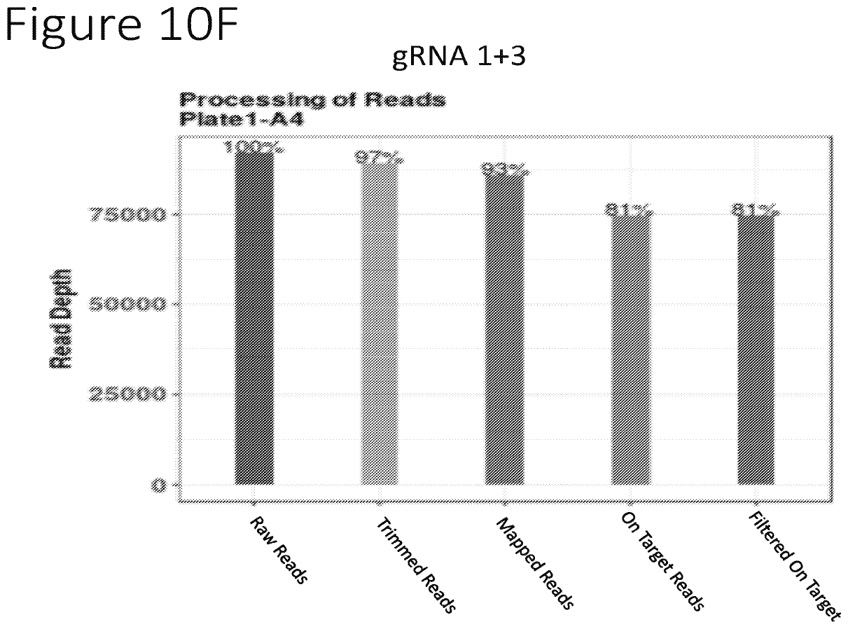

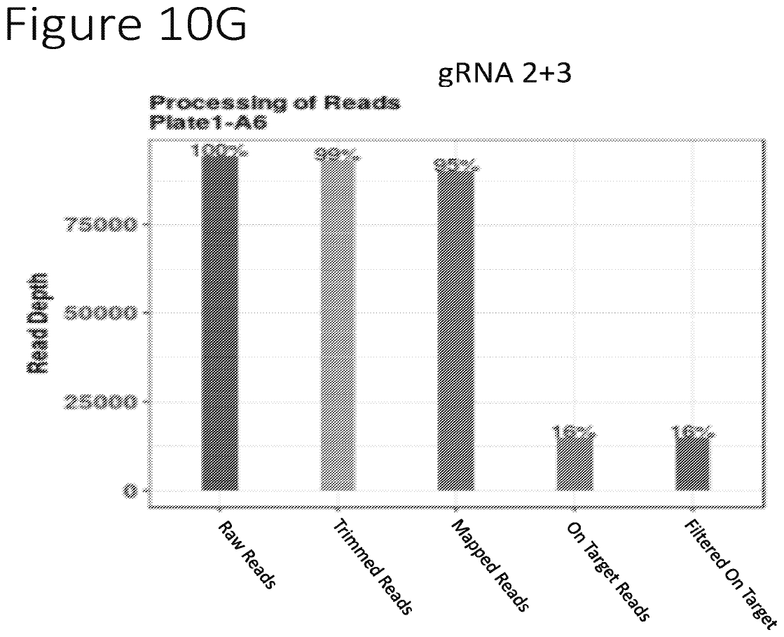

Flow cytometry analysis of TGFBR2 expression is shown in FIGS. 9A-9G. FIG. 9A shows control data in which NK cells were exposed to mock CRISPr/Cas9 gene editing conditions (nonsense or missing guide RNA). As shown, about 21% of the NK cells are positive for TGFBR2 expression. When the CRISPr/Cas9 machinery was guided using guide RNA 1 (SEQ ID NO. 147) TGFBR2 expression was not reduced (see FIG. 9B). Similar results are shown in FIGS. 9C and 9D, where guide RNA 2 (SEQ ID NO. 148) and guide RNA 3 (SEQ ID NO. 149) used individually had limited impact on TGFRB2 expression. In contrast, combinations of guide RNAs resulted in reduced TGFBR2 expression. FIG. 9E shows results from the combination of guide RNA 1 (SEQ ID NO. 147) and guide RNA 2 (SEQ ID NO. 148) and FIG. 9F shows expression of TGFBR2 after use of the combination of guide RNA 1 (SEQ ID NO. 147) and guide RNA 3 (SEQ ID NO. 149). In each case, TGFBR2 expression was reduced by ˜50% as compared to the use of the guide RNAs alone (˜11-12% expression). FIG. 9G shows a marked reduction in TGFBR2 expression when both guide RNA 2 (SEQ ID NO. 148) and guide RNA 3 (SEQ ID NO. 149), with only ˜1% of the NK cells expressing TGFBR2. Next Generation Sequencing was used to confirm the flow cytometry expression analysis. These data are shown in FIGS. 10A-10G, which correspond to the respective guide RNAs in FIGS. 9A-9G. These data confirm that guide RNAs used with a CRISPr/Cas system can reduce expression of a specific target molecule, such as TGFBR2 on NK cells. According to several embodiments, a combination of guide RNAs, such as TGFBR guide RNA 2 and guide RNA 3 work synergistically together to essentially eliminate expression of the TGFBR2 by NK cells.

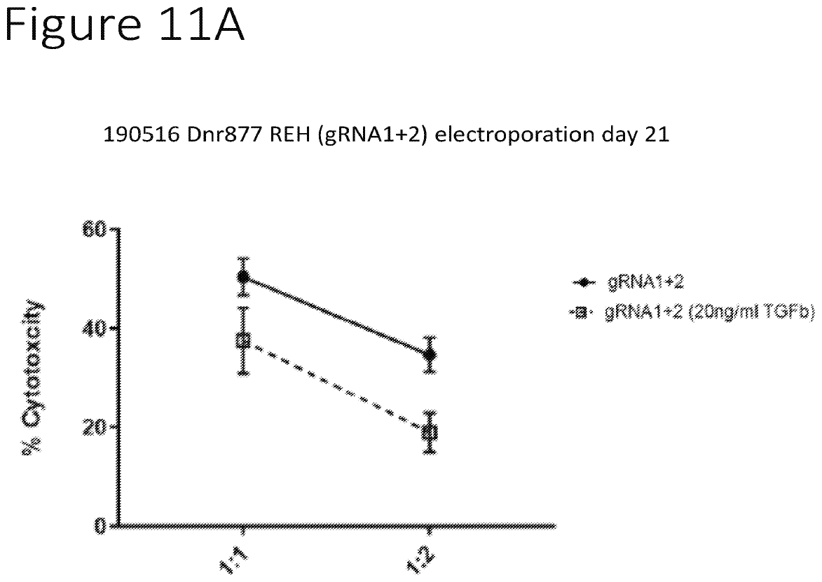

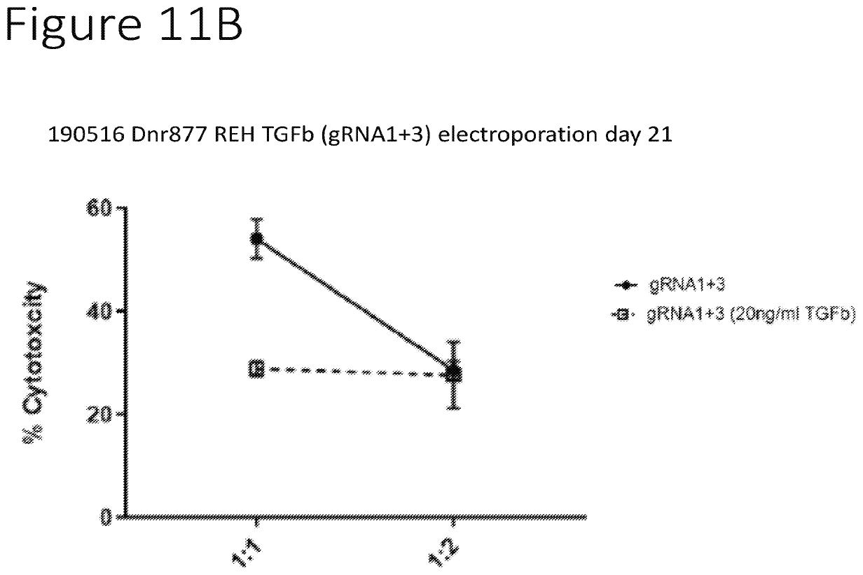

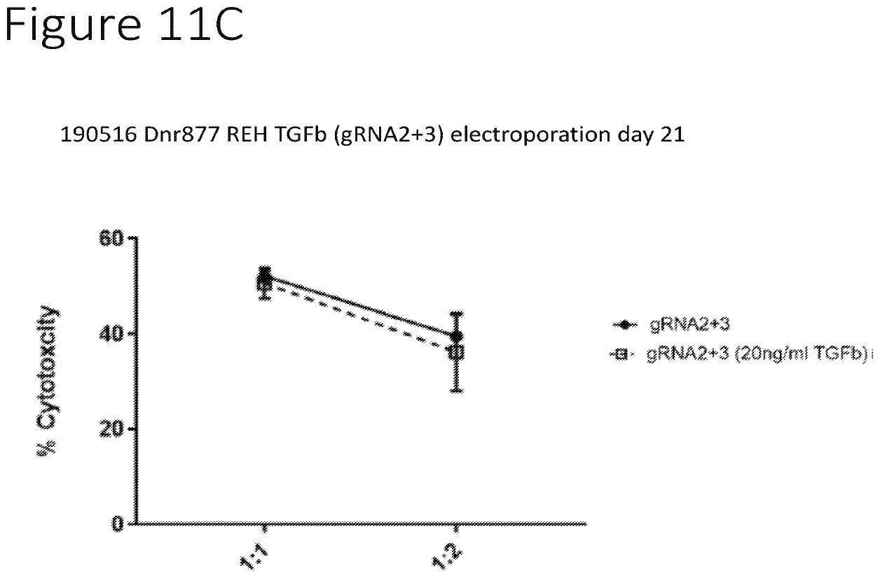

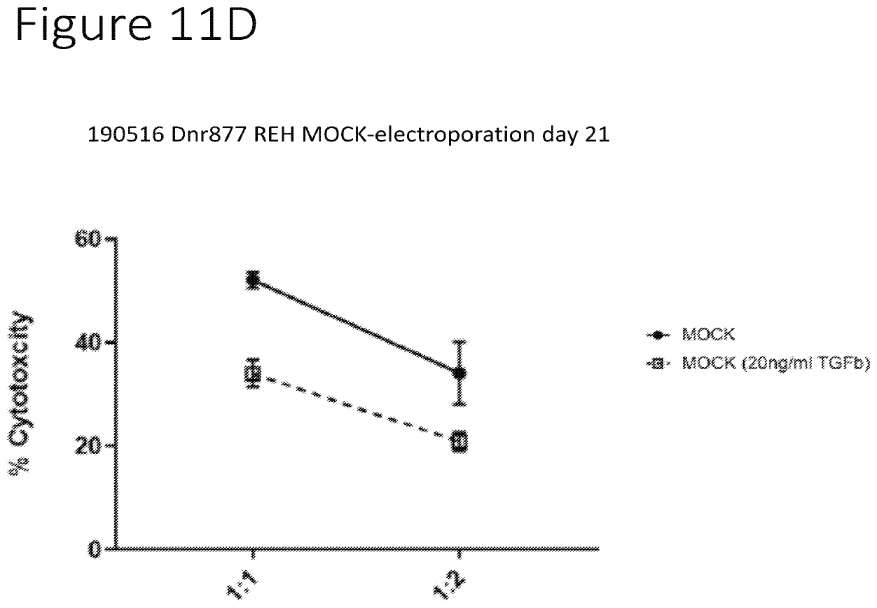

Building on these expression knockout experiments, the ability of TGFb to inhibit the cytotoxicity of TGFBR2 knockout NK cells was evaluated. To do so, NK cells were subject to TGFBR2 gene editing as discussed above, and at 21 days post-electroporation with the gene editing machinery, the cytotoxicity of the resultant cells was evaluated against REH tumor cells at 1:1 and 1:2 effector:target ratios and in the absence (closed circles) or presence of TGFb (20 ng/mL; open squares). Data are summarized in FIGS. 11A-11D. FIG. 11A shows data related to the combination of guide RNA 1 and 2. As evidenced by the decrease in the detected percent cytotoxicity at both 1:1 and 1:2 ratios with the addition of TGFb, these data are in line with the expression data discussed above, in that the presence of TGFBR2 (due to limited reduction in the expression of the receptor) allows TGFb to inhibit the cytotoxic activity of the NK cells. FIG. 11D shows mock results, with a similar cytotoxicity pattern to that shown in FIG. 11A. FIG. 11B shows similar data in that the presence of TGFb reduced the cytotoxicity of NK cells at a 1:1 target ratio when guide RNAs 1 and 3 were used to knock down TGFBR2 expression. At a 1:2 target ratio, the NK cells exhibited the same degree of cytotoxicity (reduced as compared to TGFBR2 knock down NK cells alone) whether TGFb was present or not. In contrast to the other experimental conditions, and in line with the expression data, FIG. 11C shows the cytotoxicity of NK cells edited with CRISPr using both guide RNAs 2 and 3. Despite the presence of TGFb at concentrations that reduced the cytotoxicity of the other NK cells tested, these NK cells that essentially lack TGFBR2 expression due to the gene editing show negligible fall off in cytotoxicity. These data show that, according to several embodiments, disclosed herein, use of gene editing techniques to disrupt, for example, expression of a negative regulator of immune cell activity results in an enhanced cytotoxicity and/or persistence of immune cells as disclosed herein.

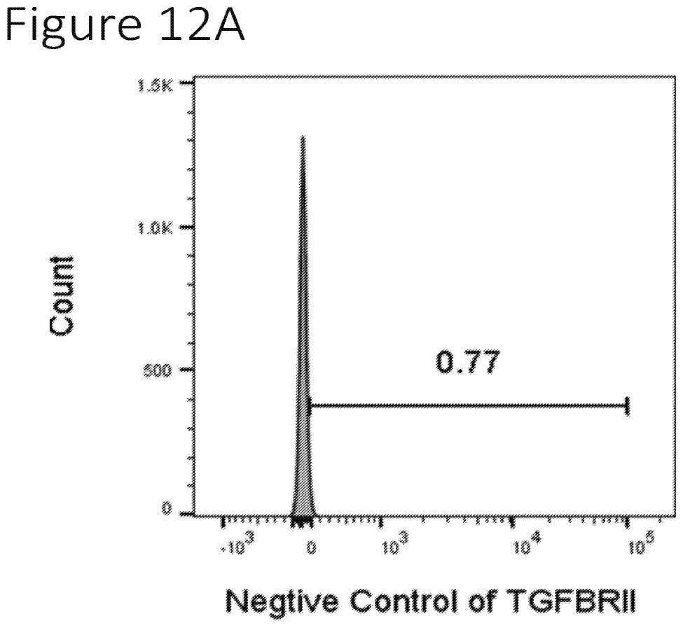

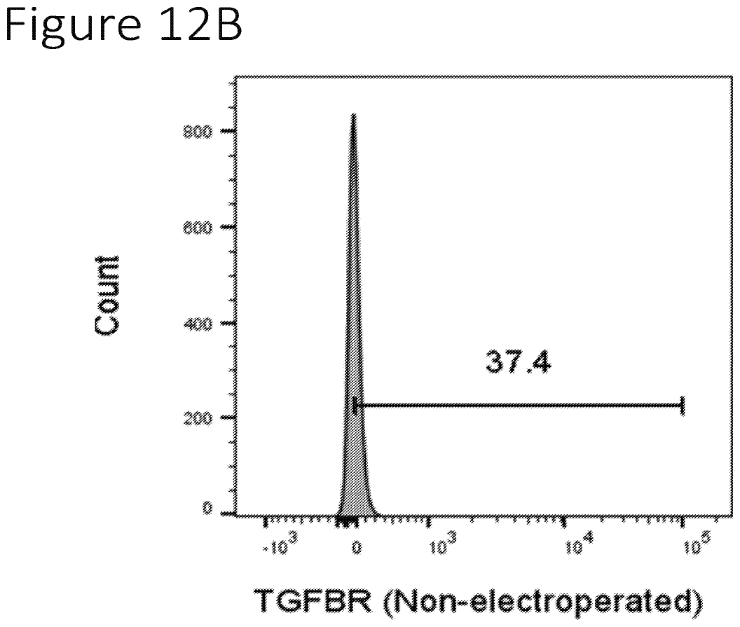

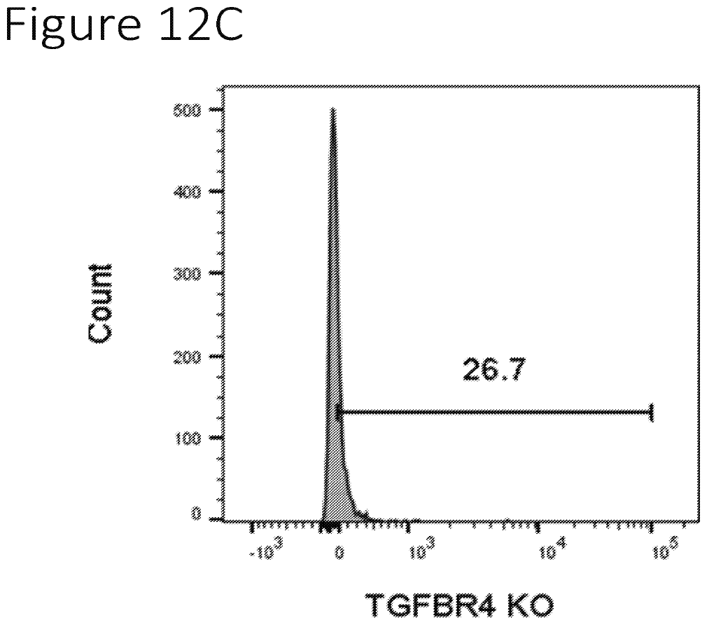

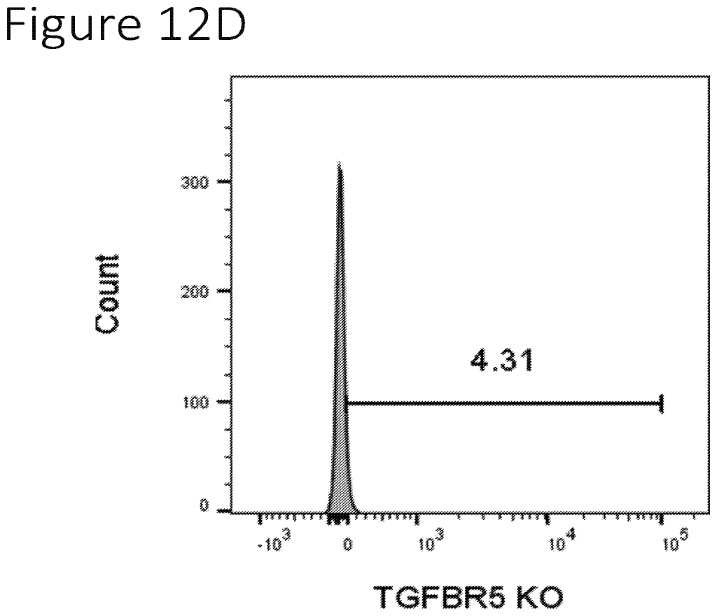

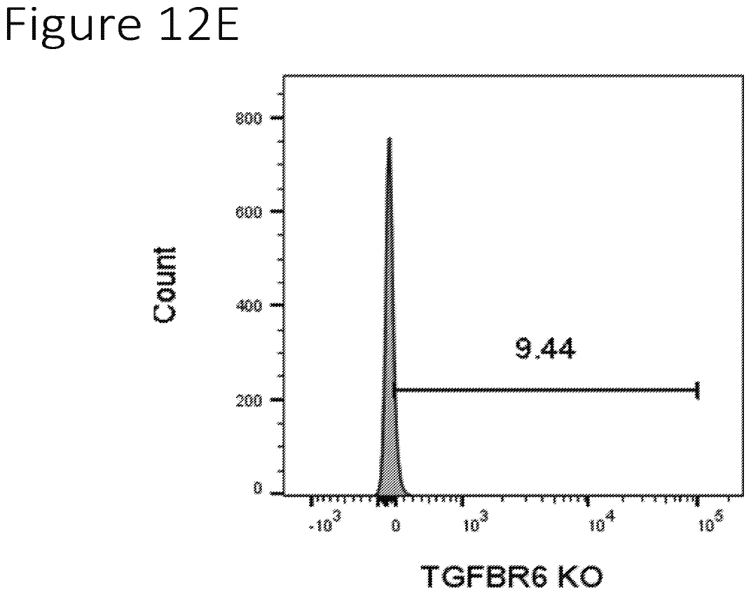

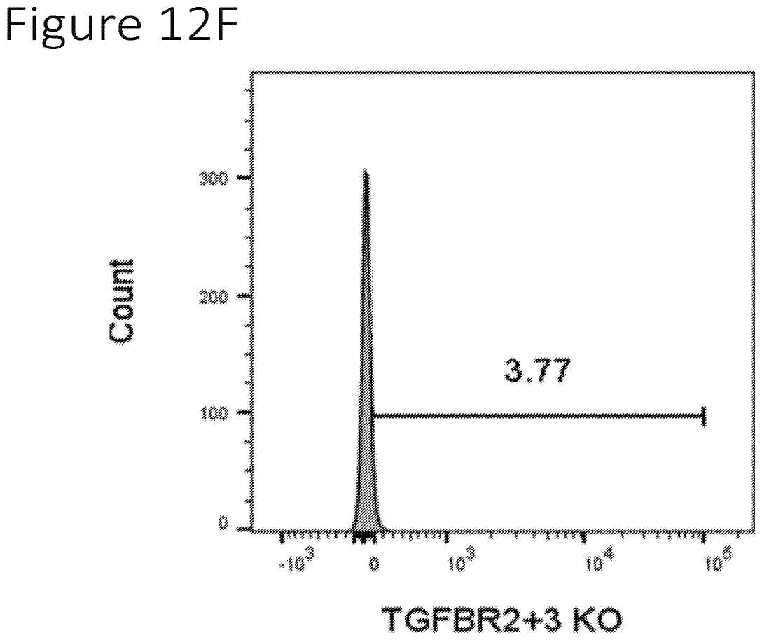

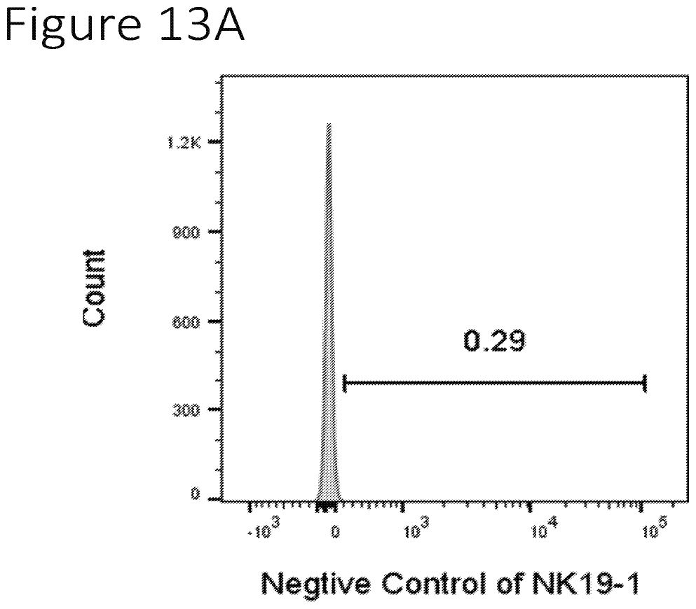

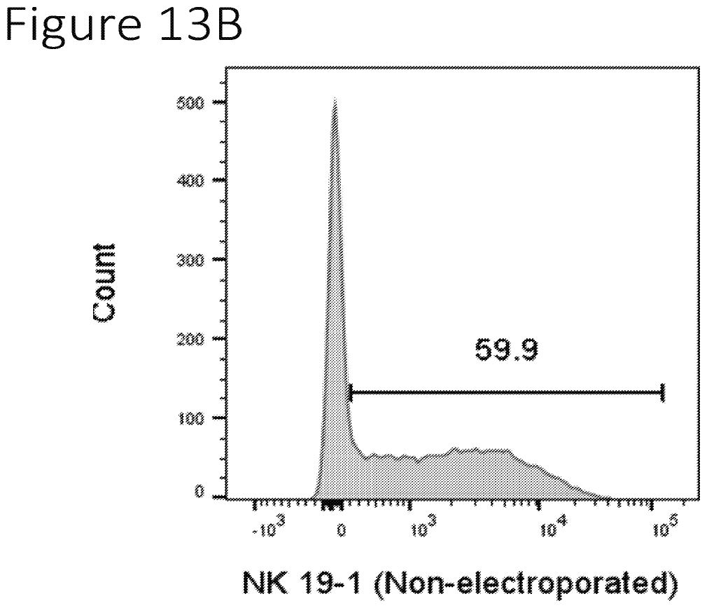

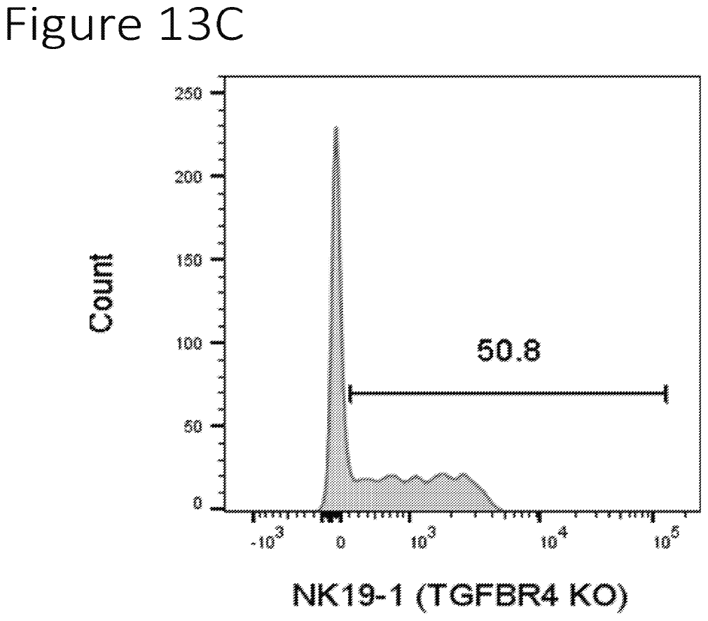

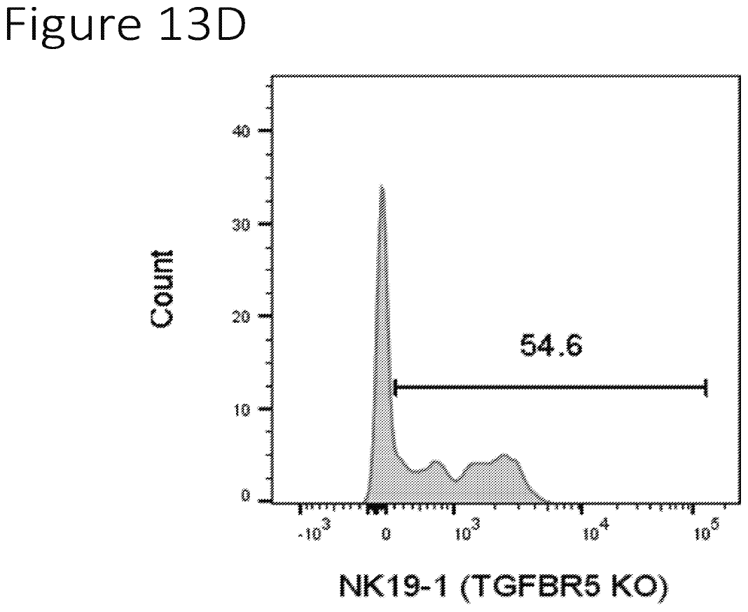

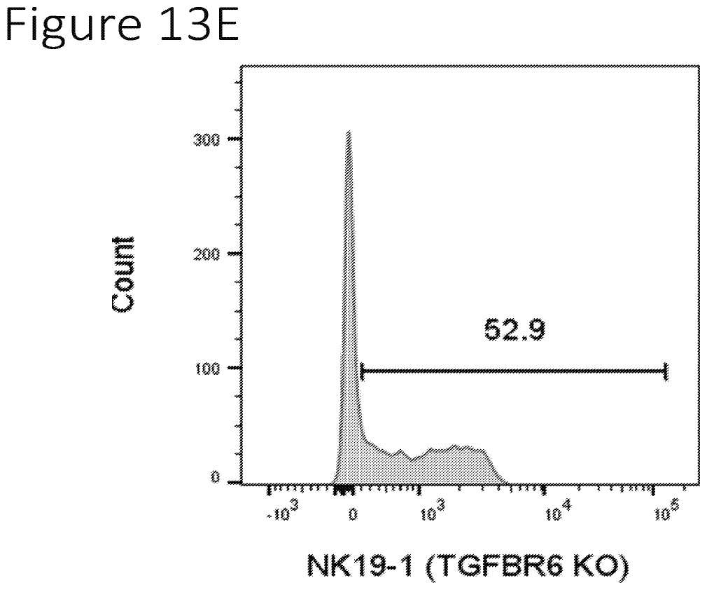

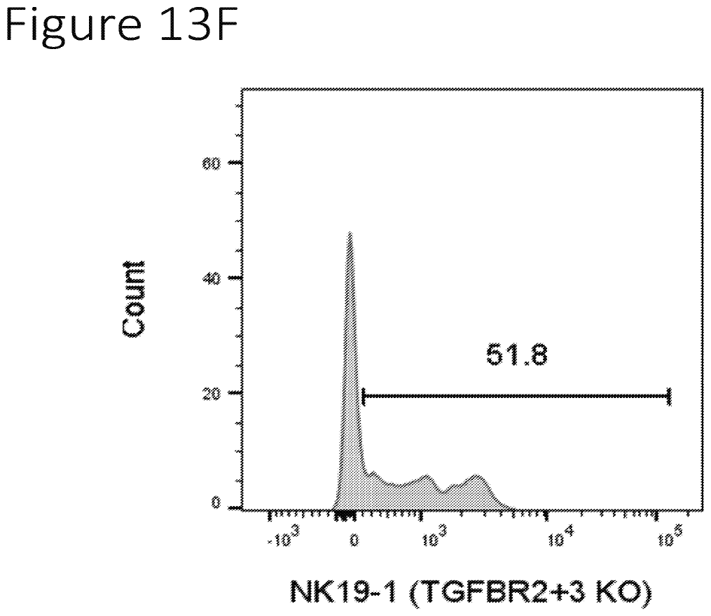

FIGS. 12A-12F present flow cytometry data related to additional guide RNAs directed against TGFBR2 (see table 1). FIG. 12A shows negative control evaluation of expression of TGFBR2 by NK cells (e.g., NK cells not expressing TGFBR2). FIG. 12B shows positive control data for NK cells that were not electroporated with CRISPr/Cas9 gene editing machinery, thus resulting in ˜37% expression of TGFBR2 by the NK cells. FIGS. 12C, 12D and 12E show TGFBR2 expression by NK cells that were subject to CRISPr/Cas9 editing and guided by guide RNA 4 (SEQ ID NO. 150), guide RNA 5 (SEQ ID NO. 151), or guide RNA 6 (SEQ ID NO. 152), respectively. Guide RNA 4 resulted in modest knock down of TGFBR2 expression (˜10% reduced compared to positive control). In contrast, guide RNA 5 and guide RNA 6 each reduced TGFBR2 expression significantly, by about 33% and 28%, respectively. These two single guide RNAs were on par the with the reduction seen (discussed above) with the combination of guide RNA 2 and guide RNA 3 (additional data shown in FIG. 12F. In accordance with several embodiments discussed herein, engineered immune cells are subjected to gene editing, such that the resultant immune cell is engineered to express a chimeric construct that imparts enhanced cytotoxicity to the engineered cell. In addition, such cells are genetically modified, for example to dis-inhibit the immune cells by disrupting at least a portion of an inhibitory pathway that functions to decrease the activity or persistence of the immune cell. To confirm that gene editing and expression of cytotoxic constructs are compatible, as disclosed herein, expression of a non-limiting example of a chimeric antigen receptor construct targeting CD19 (here identified as NK19-1) was evaluated subsequent to gene editing to knock down TGFBR2 expression. These data are shown in FIGS. 13A-13F

FIG. 13A shows a negative control assessment of expression of a non-limiting example of an anti-CD19 directed CAR (NK19-1). Here, NK cells were not transduced with the NK19-1 construct. In contrast, FIG. 13B shows positive control expression of NK19-1 by non-electroporated NK cells (as a control to account for lack of processing through a CRISPr gene-editing protocol. FIG. 13C shows the expression of NK19-1 by NK cells that were subject to TGFBR2 knock down through the use of CRISPr/Cas9 and guide RNA 4. As shown, there is only a nominal reduction in NK19-1 expression after gene editing with CRISPr. According to some embodiments, depending on the guide RNA and/or the mechanism for gene editing (e.g., CRISPr vs. TALEN), the slight change in CAR expression is reduced and/or eliminated. This can be seen, for example, in FIG. 13D, wherein the use of guide RNA 5 resulted in an even smaller change in NK19-1 expression by the NK cells. FIGS. 13E and 13F show data for guide RNA 6 alone, as well as guide RNA 2+3 (respectively). Taken together, these data indicate that the two approaches that are used in accordance with several embodiments disclosed herein, namely gene editing and genetic modification to induce expression of a chimeric receptor, are compatible with one another in that the process of editing the immune cell to reduce/remove expression of a negative regulator of immune cell function does not prevent the robust expression of a chimeric receptor construct. In fact, in several embodiments, gene editing and engineering of the immune cells results in a more efficacious, potent and/or longer lasting cytotoxic immune cell.

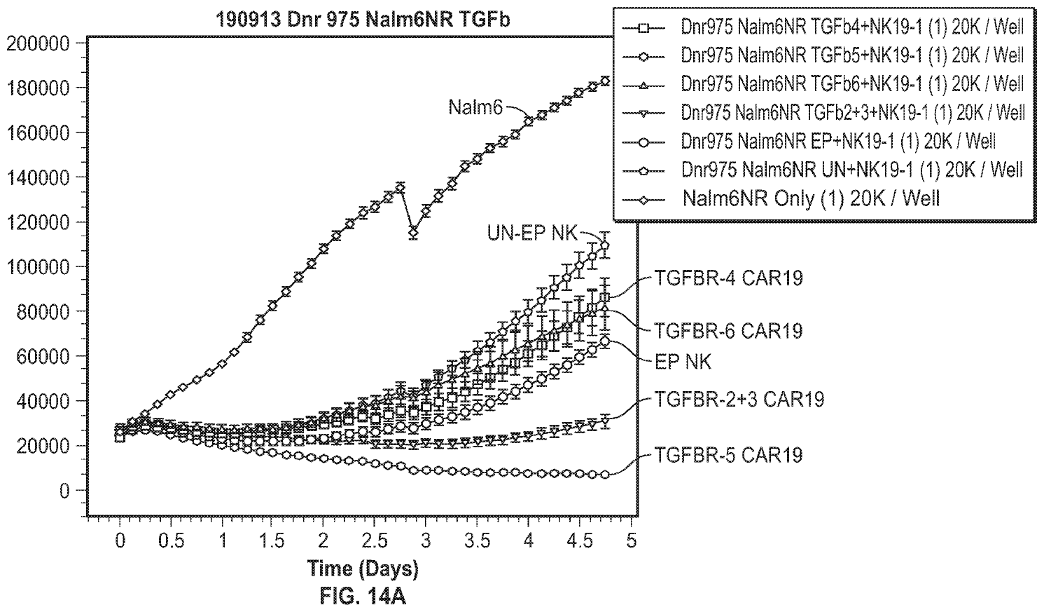

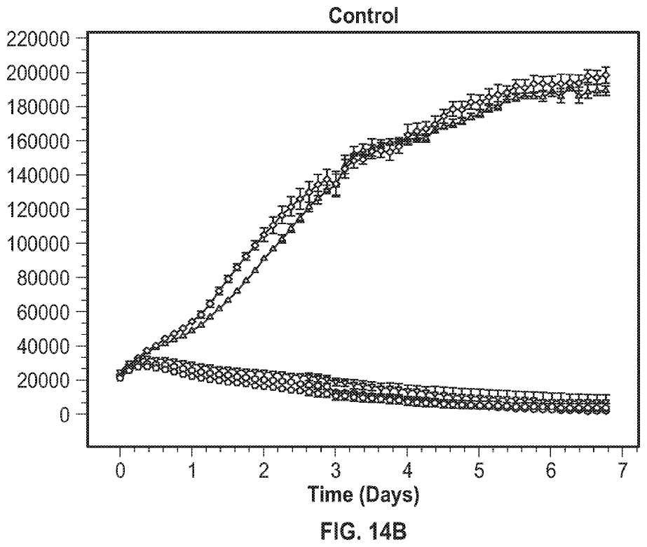

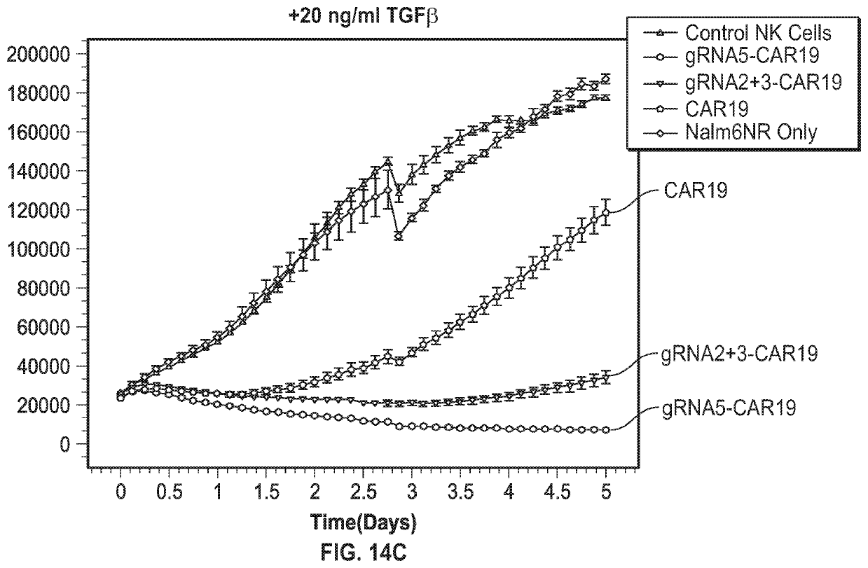

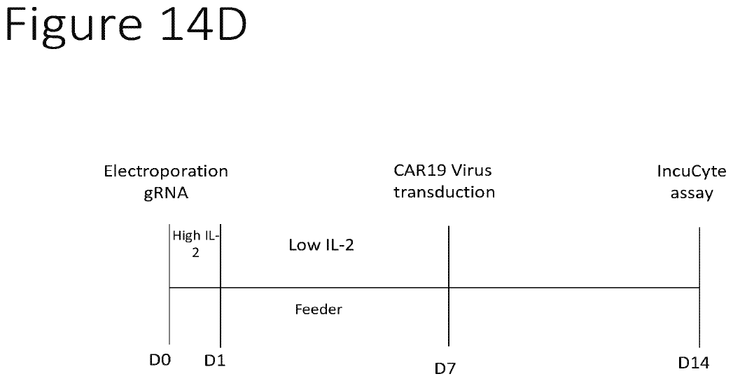

FIGS. 14A-14D show the methods and the results of an assessment of the cytotoxicity of NK cells that are subjected to gene editing (e.g., gene knockout) and/or genetic engineering (e.g., CAR expression) and their respective controls. Starting first with FIG. 14D, at Day 0, NK cells were subject to electroporation with the CRISPr/Cas9 components for gene editing, along with one (or a combination of) the indicated guide RNAs. NK cells were cultured in high-IL2 media for one day, followed by 6 additional days in culture with low IL2 and feeder cells (as discussed above). At Day 7, NK cells were transduced with the indicated anti-CD19 CAR viruses. Seven days later, the Incucyte cytotoxicity assay was performed in the presence of 20 ng/mL TGF-beta. As discussed above, TGF-beta is a potent immune suppressor that is released from the tumor cells and permeates the tumor microenvironment in vivo, in an attempt to decrease the effectiveness of immune cells in eliminating the tumor. Results are shown in FIG. 14A. As shown, in the top trace, Nalm6 cells grown alone expand robustly over the duration of the experiment. NK cells that were not electroporated (no gene editing or CAR expression; UN-EP NK) caused reduction in Nalm6 expansion. Reducing Nalm6 proliferation even further were NK cells that were subject to both gene editing and engineered CAR expression (TGFBR-4 CAR19 and TGFBR-6 CAR19). These results firstly demonstrate that these two techniques (e.g., editing and engineering) are compatible with one another and show that cytotoxicity can be enhanced in the resultant immune cells, in particular by engendering a resistance in the cells to immune suppressors in the tumor microenvironment, like TGF-beta. NK cells that were subject to electroporation, but not engineered to express a CAR (EP NK) reduced Nalm6 growth. Most notable, however, were the dramatic inhibition of Nalm6 expansion resulting from the use of NK cells engineered to express CAR19-1 (as a non-limiting example of a CAR) and which were also subject to knockout of TGFBR2 expression through either the combination of guide RNA 2 and guide RNA 3 (TGFBR-2+3 CAR19) or through the use of the single guide RNA, guide RNA 5 (TGFBR-5 CAR19). These data further evidence that, according to several embodiments disclosed herein, there a robust enhancement of the cytotoxicity of immune cells can be realized through a synergistic combination of reducing an inhibitory pathway (e.g., reduction in the inhibitory effects of TGFb by knockout of the TGFBR2 on immune cells through gene editing) and introducing a cytotoxic signaling complex (e.g., through engineering of the cells to express a CAR). FIGS. 14B and 14C show control data and selected data from FIG. 14A, respectively. FIG. 14B shows the significant cytotoxic effects of all constructs tested against Nalm6 cells alone (e.g., not recapitulating the immune suppressive effect of the tumor microenvironment). Each construct tested effectively eliminated tumor cell growth. In FIG. 14C, the tumor challenge experiments were performed in the presence of 20 ng/mL of TGF-beta to recapitulate the tumor microenvironment. FIG. 14C is selected data from 14A, to show the effects of gene editing to knockout the TGFB2 receptor more clearly. Cells engineered to express NK19-1 (as a non-limiting example of a CAR) showed the ability to reduce tumor growth as compared to controls. However, NK cells expressing NK19-1 and engineered (through CRISPR/Cas9 gene editing and the use of the non-limiting examples of guide RNAs) showed even more significant reductions in growth of tumor cells. Thus, according to several embodiments, leading to results such as those shown in FIG. 14A (and 14C), these gene editing techniques can be used to enhance the cytotoxicity of NK cells, even in the immune suppressive tumor microenvironment. In several embodiments, analogous techniques can be used on T cells. Additionally, in several embodiments, analogous approaches are used on both NK cells and T cells. Further, in additional embodiments, gene editing is used to engender edited cells, whether NK cells, T cell, or otherwise, resistance to one or more immune suppressors found in a tumor microenvironment.

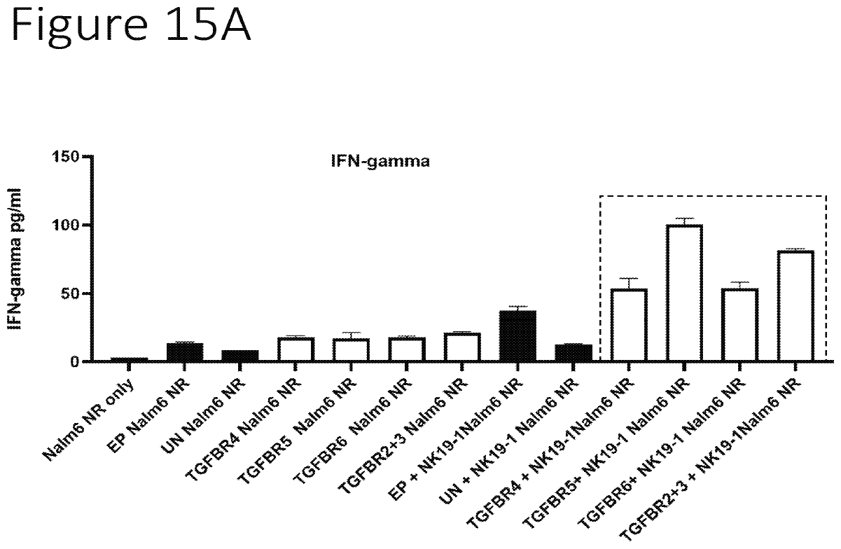

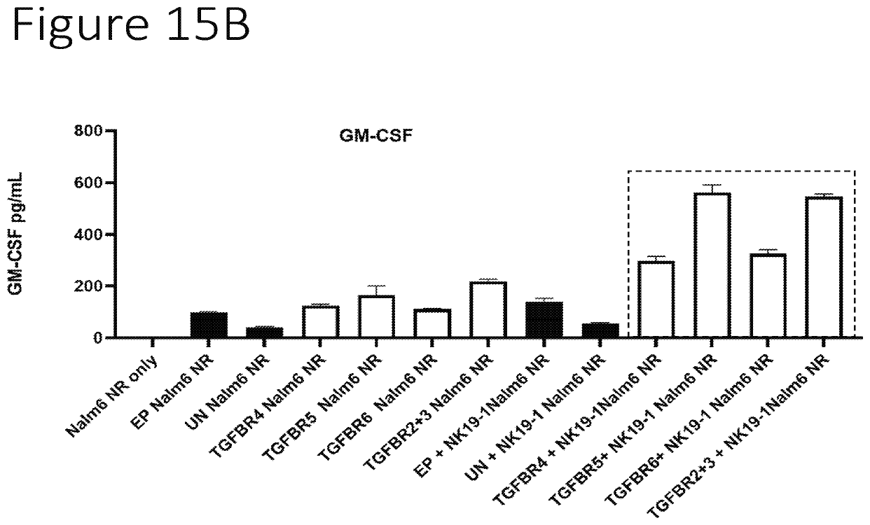

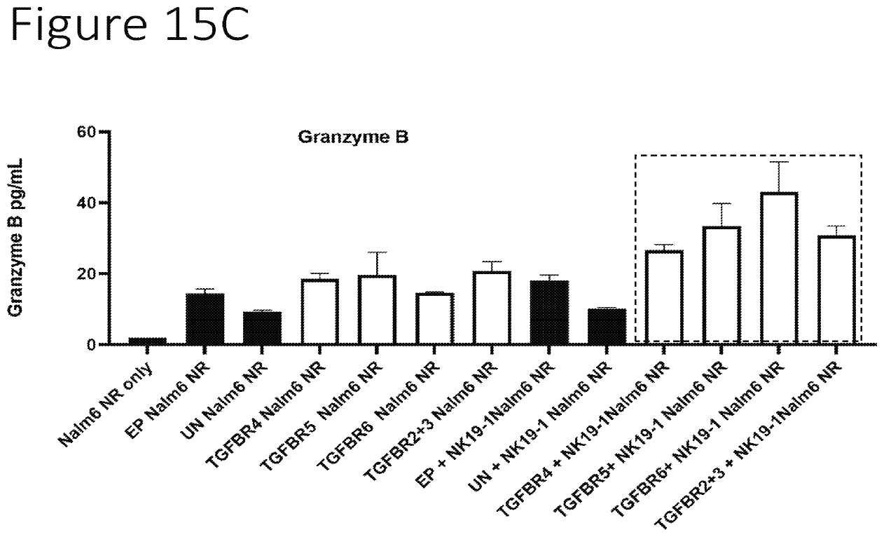

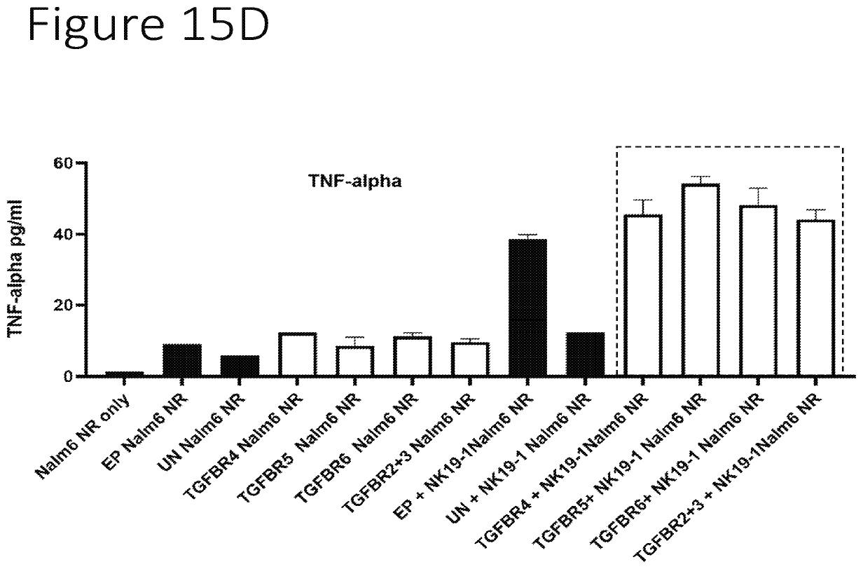

To evaluate the potential mechanisms by which the modified immune cells exert their increased cytotoxic activity the cytokine release profile of each of the types of cells tested was evaluated, the data being shown in FIGS. 15A-15D. In brief, each of the NK cell groups were treated with TGFb 1 at a concentration of 20 ng/mL overnight prior inception of the cytotoxicity assay. The NK cells were washed to remove TGFb prior to co-culture of the NK cells with Nalm6 tumor cells. NK cells were co-cultured with Nalm6 tumor cells expressing nuclear red fluorescent protein (Nalm6-NR) at an E:T ratio of 1:1 (2×10⁴ effector: 2×10⁴ target cells). Cytokines were measured by Luminex assay. As shown in FIG. 15A, there was a modest increase in the release of IFNg when TGFBR2 expression was reduced by gene editing (see for example the histogram bar for “TGFBR2+3 Nalm6 NR”). Introduction of the anti-CD19 CAR induced a substantial increase in IFNg production (EP+NK19-1 Nalm6-NR). Most notably, however, are the last four groups shown in FIG. 15A (see dashed box), which represent the use of either single guide RNAs, or a combination of guide RNAs, to direct the CRISPr/Cas9-mediated knockdown of expression of the TGFBR2 in combination with the expression of an anti-CD19 CAR. The release of these increased amounts of IFNg are, at least in part, responsible for the enhanced cytotoxicity seen using these doubly-modified immune cells. Similar to IFNg, GM-CSF release was significantly enhanced in these groups. GM-CSF can promote the differentiation of myeloid cells and also as an immunostimulatory adjuvant, thus it's increased release may play a role in the increased cytotoxicity seen with these cells. Similar patterns are seen when assessing the release of Granzyme B (a potent cytotoxic protein released by NK cells) and TNFalpha (another potent cytokine). These data further evidence that increased release of various cytokines are at play in causing the substantial increase in cytotoxicity seen with the gene edited and genetically modified immune cells, as in accordance with several embodiments disclosed herein, as the gene editing aids in resisting immune suppressive effects that would be seen in the tumor microenvironment.

In accordance with additional embodiments, a disruption of, or elimination of, expression of a receptor, pathway or protein on an immune cell can result in the enhanced activity (e.g., cytotoxicity, persistence, etc.) of the immune cell against a target cancer cell. In several embodiments, this results from a disinhibition of the immune cell. Natural killer cells, express a variety of receptors, such particularly those within the Natural Killer Group 2 family of receptors. One such receptor, according to several embodiments disclosed herein, the NKG2D receptor, is used to generate cytotoxic signaling constructs that are expressed by NK cells and lead to enhanced anti-cancer activity of such NK cells. In addition, NK cells express the NKG2A receptor, which is an inhibitory receptor. One mechanism by which tumors develop resistance to immune cells is through the expression of peptide-loaded HLA Class I molecules (HLA-E), which suppresses the activity of NK cells through the ligation of the HLA-E with the NKG2A receptor. Thus, while one approach could be to block the interaction of the HLA-E with the expressed NKG2A receptors on NK cells, according to several embodiments disclosed herein, the expression of NKG2A is disrupted, which short circuits that inhibitory pathway and allows enhanced NK cell cytotoxicity.

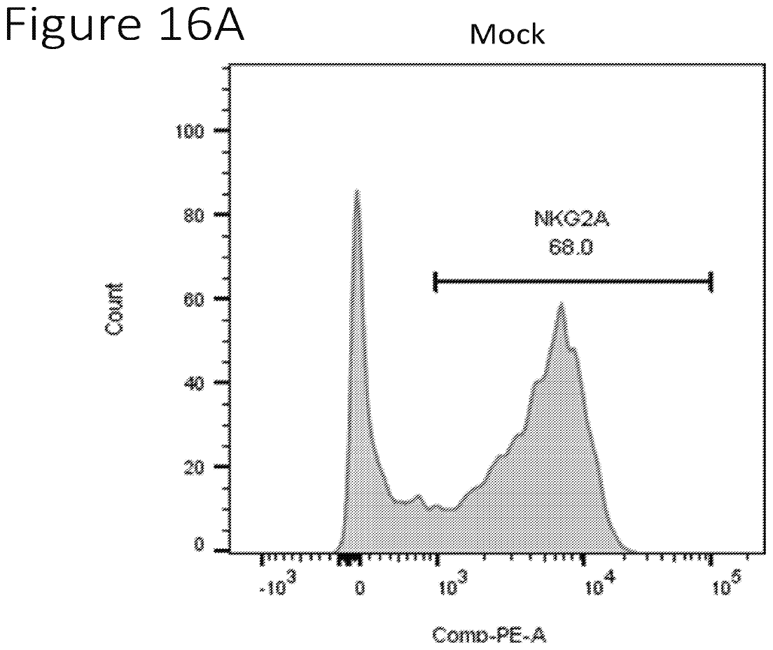

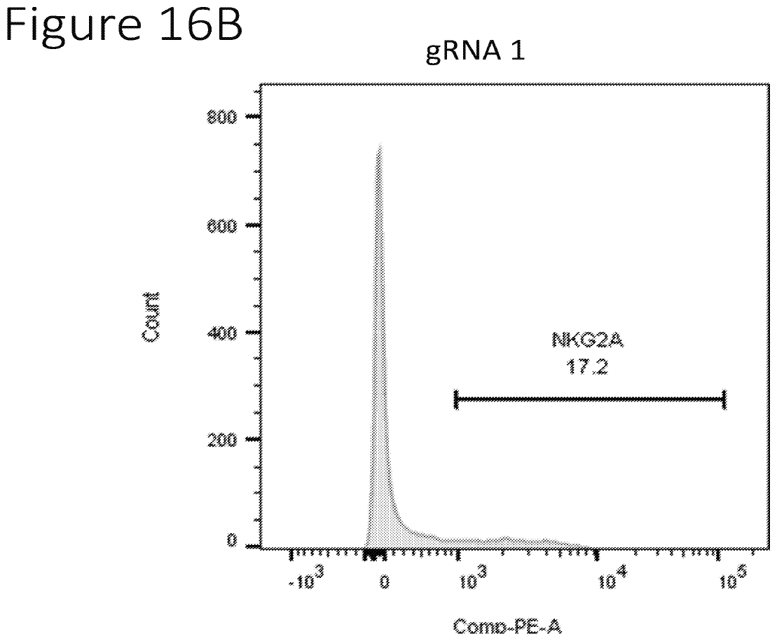

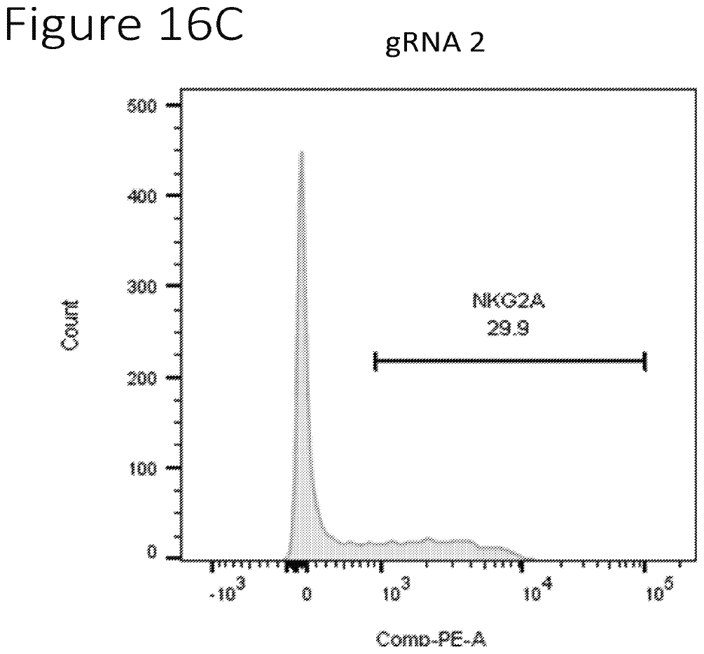

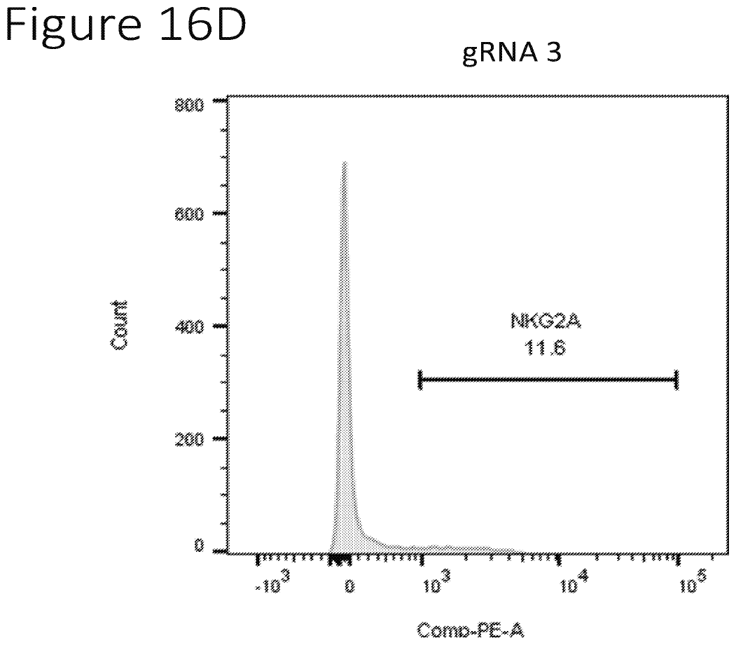

FIGS. 16A-16D show data related to the disruption of expression of NKG2A expression by NK cells. As discussed above with TGFBR2, CRISPr/Cas9 was used to disrupt NKG2A expression using the non-limiting examples of guide RNAs show below in Table 2.

TABLE 2
NKG2A Guide RNAs

| SEQ ID NO: | Name | Sequence | Target |
|---|---|---|---|
| 158 | NKG2A-1 | GGAGCTGATGGTAAATCTGC | Exon 4 |
| 159 | NKG2A-2 | TTGAAGGTTTAATTCCGCAT | Exon 3 |
| 160 | NKG2A-3 | AACAACTATCGTTACCACAG | Exon 4 |

FIG. 16A shows control NKG2A expression by NK cells, with approximately 70% of the NK cells expressing NKG2A. FIG. 16B demonstrates that significant reductions in NKG2A expression can be achieved, with the use of guide RNA 1 reducing NKG2A expression by over 50%. FIG. 16C shows a more modest reduction in NKG2A expression using guide RNA 2, with just under 30% of the NK cells now expressing NKG2A. FIG. 16D shows that use of guide RNA 3 provides the most robust disruption of NKG2A expression by NK cells, with only ˜12% of NK cells expressing NKG2A.

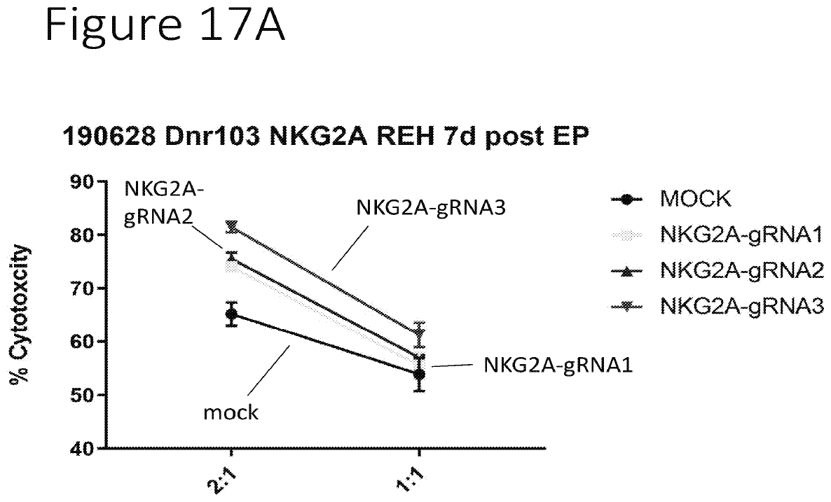

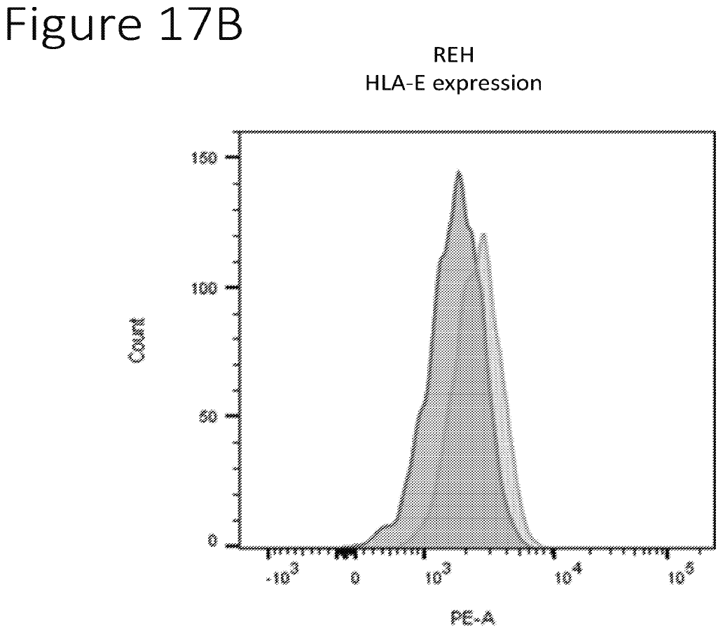

FIG. 17A shows summary cytotoxicity data related to the NK cells with reduced NKG2A expression against Reh tumor cells at 7 days post-electroporation with the gene editing machinery. NK cells were tested at both a 2:1 E:T and a 1:1 E:T ratio. At 1:1 E:T, each of the gene edited NK cell types induced a greater degree of cytotoxicity than the mock NK cells. The improved cytotoxicity detected with guide RNA 1 and guide RNA 2 treated NK cells were slightly enhanced over mock. The guide RNA that induced the greatest disruption of NKG2A expression on NK cells also resulted in the greatest increase of cytotoxicity as compared to mock (see 1:1 NKG2A-gRNA3). At a 2:1 ratio, each of the modified NK cell types significantly outperformed mock NK cells. As with the lower ratio, NK cells edited using guide RNA3 to target the CRISPr/Cas9 showed the most robust increase in cytotoxicity, an inverse relationship with the degree of NKG2A expression disruption. As discussed above, the interaction of HLA-E on tumor cells with the NKG2A on NK cells, absent intervention, can inhibit the NK cell activity. FIG. 17B confirms that Reh tumor cells do in fact express HLA-E molecules, and therefore, in the absence of the gene editing to disrupt NKG2A expression on the NK cells, would have been expected to inhibit NK cell signaling (as seen with the Mock NK cell group in FIG. 17A).

While the disruption of the HLA-E/NKG2A interaction had a clear positive impact on cytotoxicity of NK cells, other pathways were investigated that may impact immune cell signaling. One such example is the CIS/CISH pathway. Cytokine-inducible SH2-containing protein (CIS) is a negative regulator of IL-15 signaling in NK cells, and is encoded by CISH gene in humans. IL-15 signaling can have positive impacts on the NK cell expansion, survival, cytotoxicity and cytokine production. Thus, a disruption of CISH could render NK cells more sensitive to IL-15, thereby increasing their anti-tumor effects.

As discussed above, CRISPr/CAs9 was used to disrupt expression of CISH, though in additional embodiments, other gene editing approaches can be used. Non-limiting examples of CISH-targeting guide RNAs are shown below in Table 3.

TABLE 3
CISH Guide RNAs

| SEQ ID NO: | Name | Sequence | Target |
|---|---|---|---|
| 153 | CISH-1 | CTCACCAGATTCCCGAAGGT | Exon 2 |
| 154 | CISH-2 | CCGCCTTGTCATCAACCGTC | Exon 3 |
| 155 | CISH-3 | TCTGCGTTCAGGGGTAAGCG | Exon 1 |
| 156 | CISH-4 | GCGCTTACCCCTGAACGCAG | Exon 1 |
| 157 | CISH-5 | CGCAGAGGACCATGTCCCCG | Exon 1 |

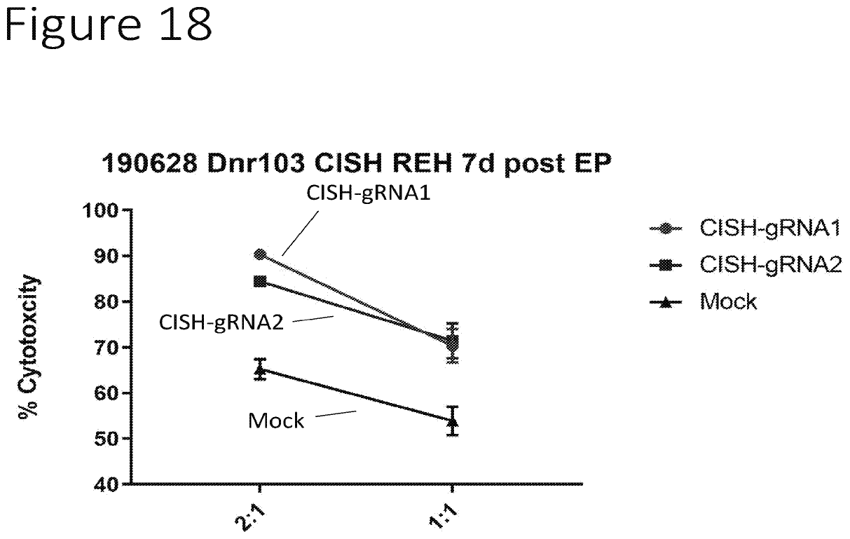

As with NKG2A knockout NK cells, CISH knockout (using guide RNA 1 or Guide RNA 2 (data not shown for CISH-3-5)) gene edited NK cells were challenged with Reh tumor cells at a 1:1 and 2:1 E:T ratio 7 days after being electroporated with the gene editing machinery. FIG. 18 shows that while mock NK cells exhibited over 50% cytotoxicity against Reh cells at 1:1, each of the gene edited NK cell groups showed nearly 20% improved cytotoxicity, with an average of ˜70% cytotoxicity against Reh cells. The enhanced cytotoxicity was even more pronounced at a 2:1 ratio. While Mock NK cells killed about 65% of Reh cells, NK cells edited with CISH guide RNA 2 killed approximately 85% of Reh cells and NK cells edited with CISH guide RNA 1 killed over 90% of Reh cells. These data clearly show that CISH knockout has a positive impact on NK cell cytotoxicity, among other positive effects as discussed above.

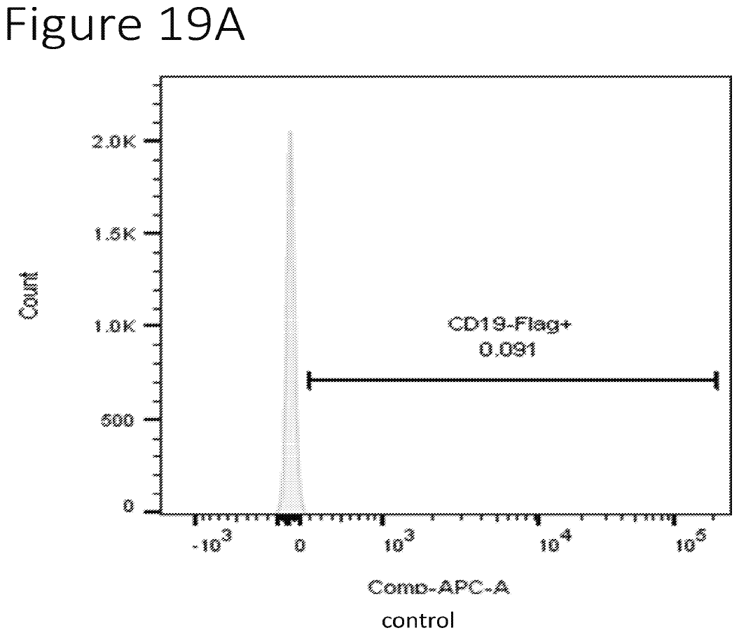

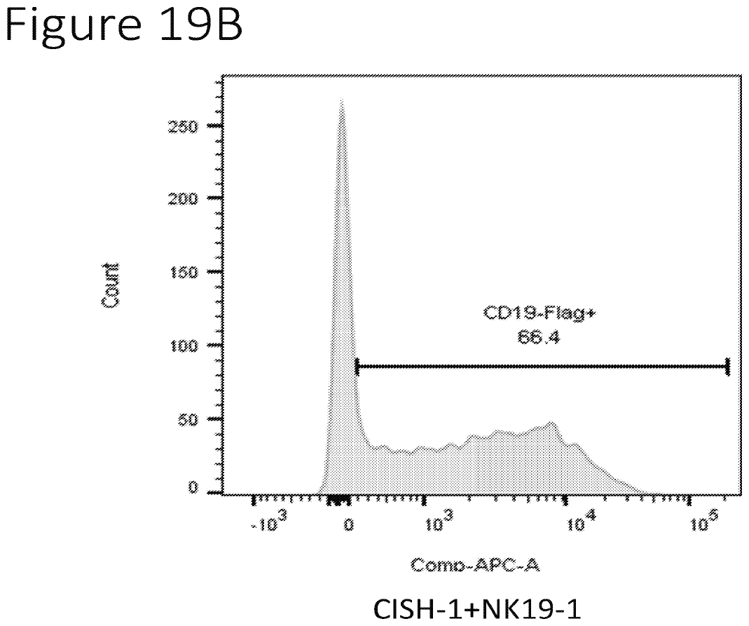

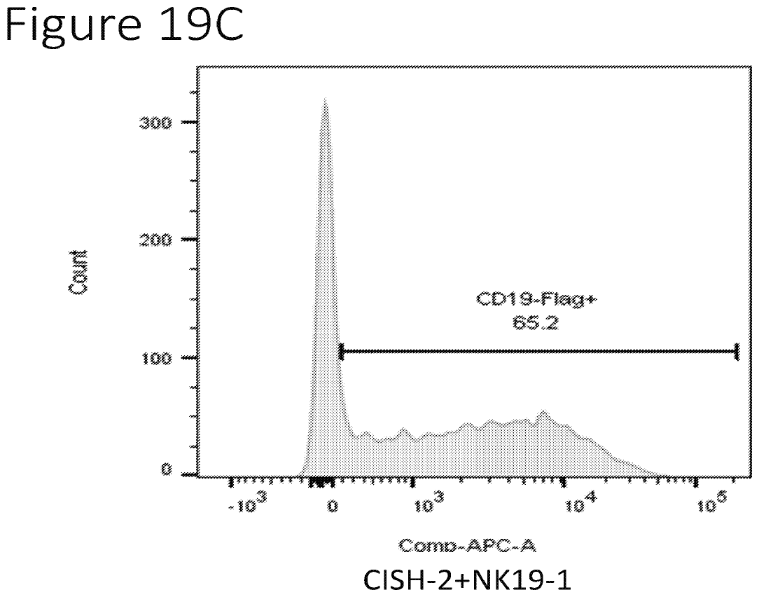

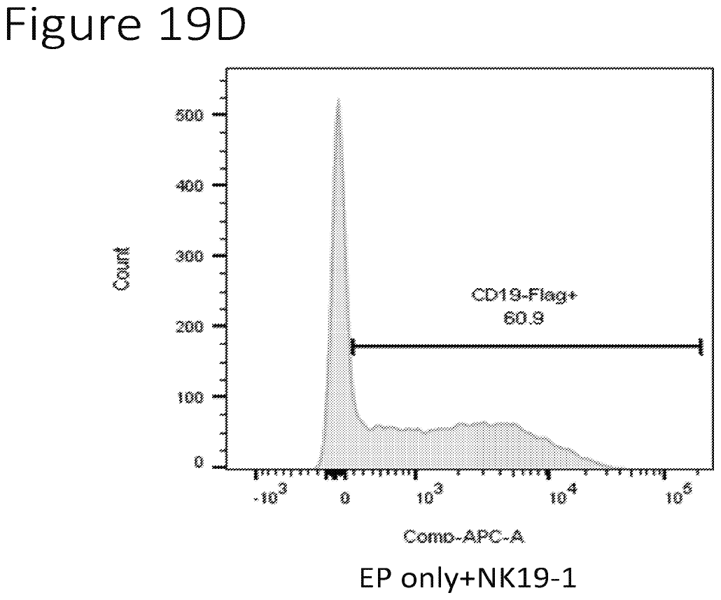

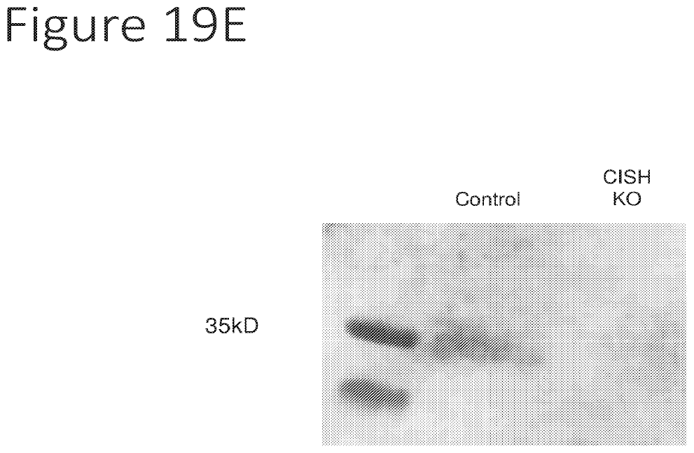

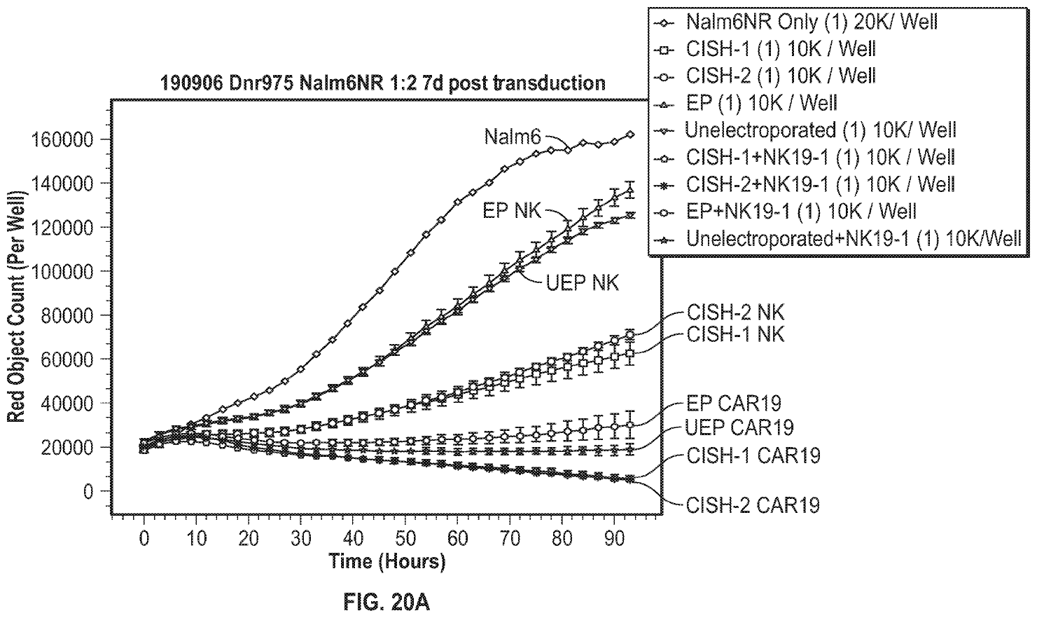

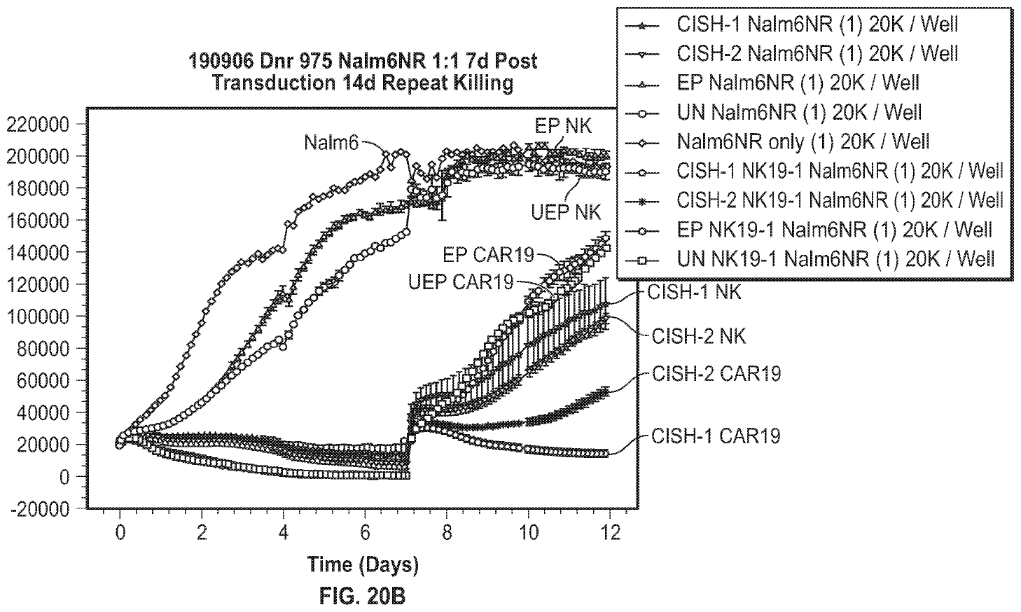

As with experiments described above, it was next evaluated whether the knockdown of CISH expression adversely impacted the ability to further modify the NK cells, for example, by transducing with a non-limiting example of a CAR (here an anti-CD19 CAR, CAR19-1). These data are shown in FIGS. 19A-19D. FIG. 19A shows negative control data for (lack of) expression of a CD19 CAR (based on detection of a Flag tag included in the CAR19-1 construct used, though some embodiments do not employ a Flag, or other, tag). FIG. 19B shows robust expression of the CD19-1 CAR by NK cells previously subjected to gene editing targeted by the CISH guide 1 RNA. FIG. 19C shows similar data for NK cells previously subjected to gene editing targeting by the CISH guide 2 RNA. FIG. 19D shows additional control data, with NK cells exposed to gene editing electroporation protocol, but without actual gene editing, thus demonstrating that the gene editing protocol itself does not adversely affect subsequent transduction of NK cells with CAR-encoding viral constructs. FIG. 20C shows a Western blot confirming the absence of expression of CIS protein (encoded by CISH) after the CISH gene editing was performed. Thus, according to some embodiments, NK cells (or T cells) are both edited, e.g., to knockout CISH expression in order to enhance one or more NK cell (T cell) characteristics through IL15-mediated signaling and are also engineered to express an anti-tumor CAR. The engineering and editing, in several embodiments, yield synergistic enhancements to NK cell function (e.g., expansion, cytotoxicity, and or persistence).

Having established that NK cells could be gene edited to reduce CISH expression and could also be engineered thereafter to express a CAR, the cytotoxicity of such doubly modified NK cells was tested. FIG. 20A shows the results of an Incucyte cytotoxicity assay where the indicated NK cell types were challenged with Nalm6 cells at a 1:2 ratio. Regarding the experimental timeline, at Day 0, NK cells were subjected to electroporation with CRISPr/Cas9, and the various CISH guide RNAs, as discussed above. The NK cells were cultured for 1 day in high IL-2 media, then moved to a low-IL-2 media where they were co-cultured with K562 cells modified to express 4-1 BB and membrane-bound IL15 for expansion. At day 7, the NK cells were transduced with the CAR19-1 viral constructs and cultured for another 7 days, with the IncuCyte cytotoxicity assay performed on Day 14.

As seen in FIG. 20A, both electroporated and un-electroporated NK cells (EP NK, UEP NK, respectively) showed nominal reduction in Nalm6 growth. When gene-edited NK cells were assessed, CISH-1 and CISH-2 NK cells both exhibited significant prevention of Nalm6 growth. Likewise, both electroporated and un-electroporated NK cells expression CAR19-1 further reduced Nalm6 proliferation. Most notably, the doubly modified CISH knockouts that express CAR19-1 exhibited complete control/prevention of Nalm6 cell growth. These results represent the synergistic activities between the two modification approaches undertaken, with gene edited CISH knockout NK cells expressing CAR19-1 showing robust anti-tumor activity, which is in accordance with embodiments disclosed herein.

These tumor-controlling effects were recapitulated in a dual challenge model as well. In this case, the experimental timeline was as described above for FIG. 20A, however, 7 days after the inception of the IncuCyte assay (performed here at 1:1 E:T), the wells were washed and re-challenged with an additional dose of Nalm6 tumor cells (20K cells per well). Data are shown in FIG. 20B. As with the single tumor cell challenge, Nalm6 cells exhibited expansion throughout the experiment, with EP and UEP NK cells allowing similar overall Nalm6 growth after the second challenge. Even with the second challenge of Nalm6 tumor cells, NK cells expression CAR19-1 constructs (EPCAR19 and UEPCAR19) curtailed Nalm6 growth more so than NK cells alone. Interestingly, with the second challenge, NK cells that were gene edited to knockout CISH expression exhibited a modestly enhanced ability to prevent Nalm6 growth as compared to those expressing CAR19-1. As discussed above, this may be due to the enhanced signaling through various metabolic pathways that are upregulated due to CISH knockout. Notably, as with the single challenge, the doubly modified NK cells that were gene edited to knockout CISH expression and engineered to express CAR19-1 showed substantial ability to prevent Nalm6 cell growth. CISH guide RNA 1 and CISH guide RNA 2 treated NK cells were on par with one another until the final stages of the experiment, where CISH guide RNA 2 treated NK cells allowed a slight increase in Nalm6 cell number. Regardless, these data show that the doubly modified NK cells possess an enhanced cytotoxic ability against tumor cells. As mentioned above, the editing coupled with engineered approach in several embodiments advantageously results in non-duplicative enhancements to NK cell function, which can synergistically enhance one or more aspects of the NK cells (such as activation, cytotoxicity, persistence etc.).

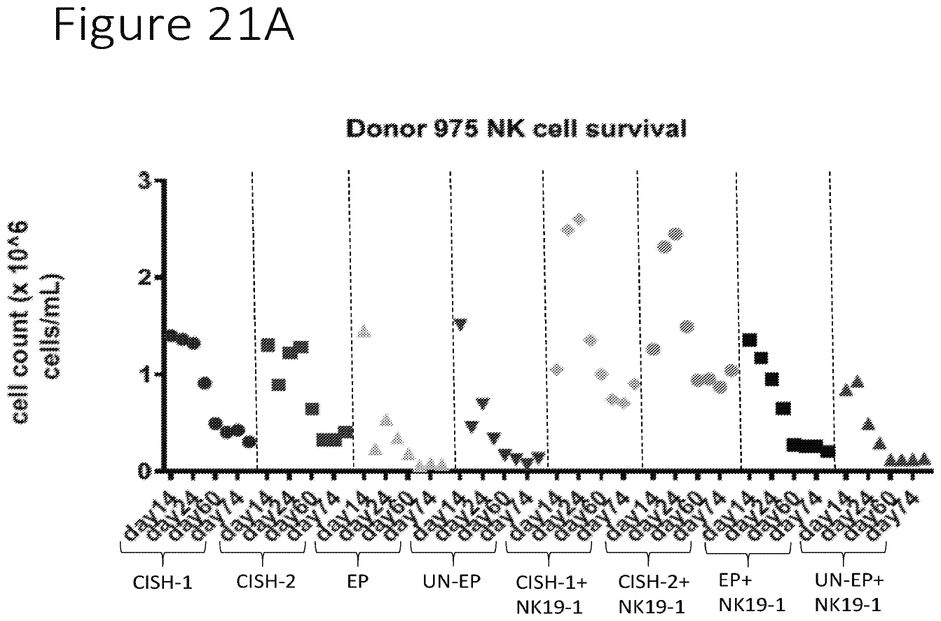

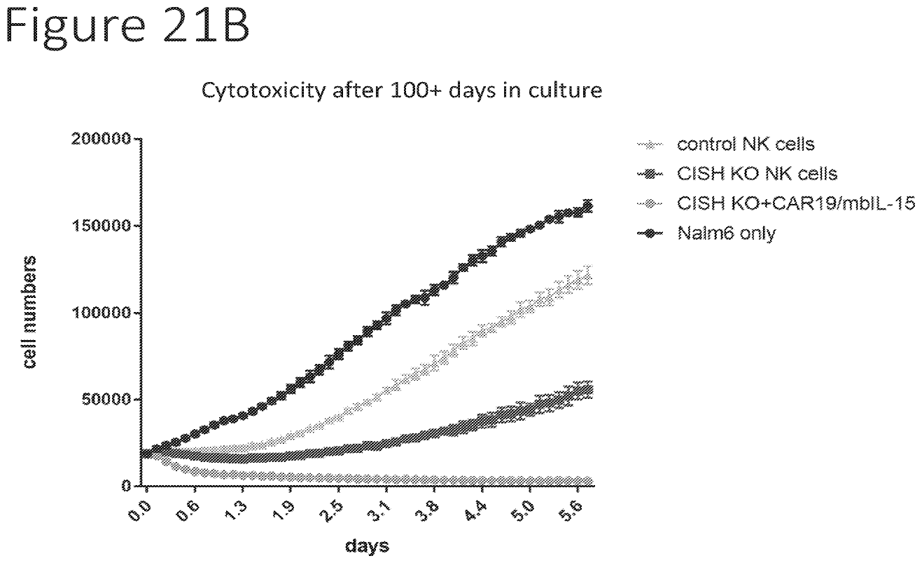

Mechanistically, without being bound by theory, it appears that the double modification of knockdown of CISH and expression of CAR19-1 allow NK cells to survive for a longer period of time, thus imparting them with an enhanced persistence against tumor cells. In several embodiments, this is due, at least in part to the enhanced signaling through various metabolic pathways in the edited cells based on knockout of CISH. Data for this analysis are shown in FIG. 21A, where cell counts were obtained for the indicated groups across 74 days in culture. Six of the eight groups tested showed a steady decline in NK cell count from about 2-3 weeks in culture, through the 74 day time point. However, the two groups of NK cells that were treated both to knockdown CISH expression and to express CAR19-1 exhibited relatively steady population size (but for a transient increase at day 24). These data suggest that the doubly modified NK cells are better able to survive than NK cells modified in only one manner (or unmodified), which may, in part, lead to their enhanced efficacy over a longer-term experiment like the secondary tumor cell challenge shown in FIG. 20B. Additionally, FIG. 21B shows cytotoxicity data for control Nalm6 cells, unmodified NK cells, CISH knockout NK cells and CISH knockout NK cells expressing CD19 CAR. This experiment was performed after each of the cell groups had been cultured for 100 days in culture. Nalm6 cells alone exhibited expansion, as expected. Control knockout NK cells (subject to electroporation only) delayed Nalm6 expansion at the initial stages, but eventually, Nalm6 cells expanded. CISH knockout NK cells showed good anti-tumor effects, with only modest increases in Nalm6 numbers at the later stages of the experiment. The cytotoxicity of NK cells at this late stage of culture is unexpected, given the growth allowed by the control NK cells. As discussed above, in several embodiments the knockout of CISH expression allows greater signaling through various ID 5 responsive pathways that lead to one or more of enhanced NK (or T) cell proliferation, cytotoxicity, and/or persistence.

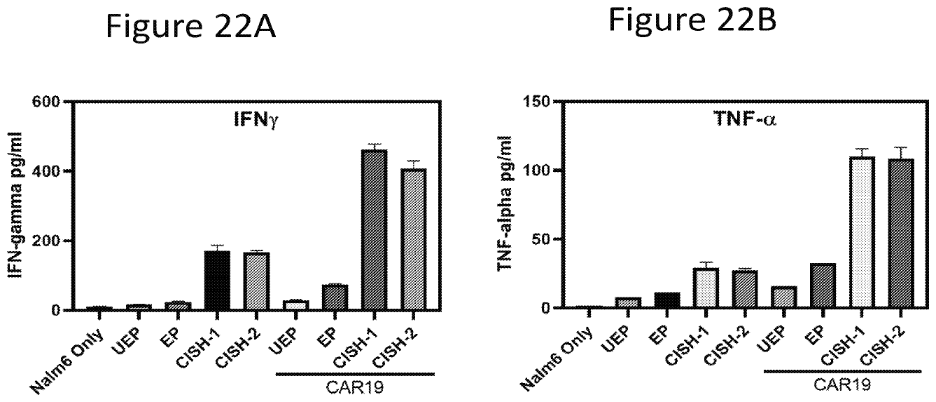

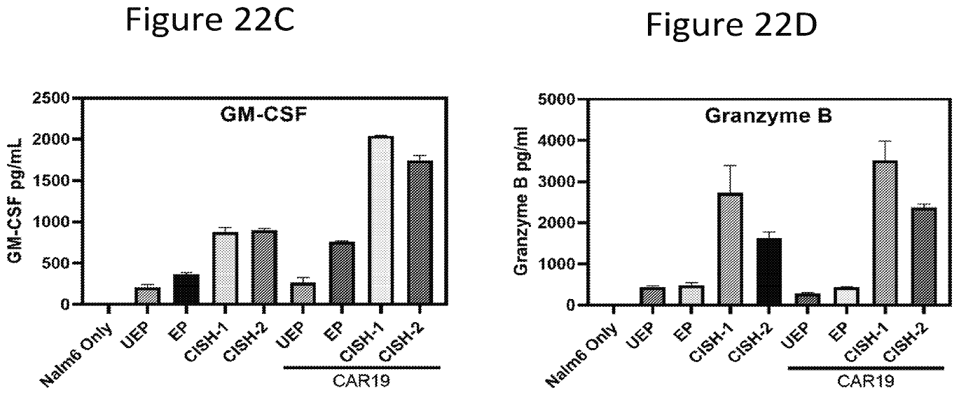

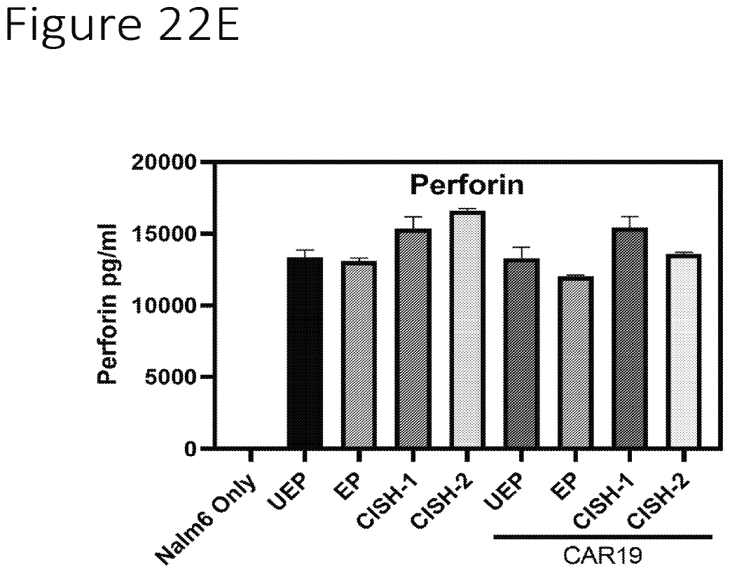

Further investigating the mechanisms by which these doubly modified cells are able to generate significant and persistent cytotoxicity, the cytokine release profiles of each group were assessed. These data are shown in FIGS. 22A-22E, with those groups of NK cells engineered to express CAR19-1 indicated by placement above the “CAR19” line on the right portion of each histogram.

FIG. 22A shows data related to IFNg production, which is notably increased when CISH is knocked out through use of CRISPr/Cas9 and either guide RNA 1 or 2 (as non-limiting embodiments of guide RNA). More interestingly, the combination of CISH knockout and CAR19-1 expression results in nearly 2.5 times more IFNg production than the CISH knockouts and 4-5 times more than any of the other groups. Similar data are shown in FIG. 22B, with respect to TNFalpha production. Likewise, while the CISH knockouts alone and the CISH-normal NKs expressing CAR19-1 release somewhat more GM-CSF, the doubly modified CISH knockout and CAR19-1-expressing NK cells show markedly increased GM-CSF release. Granzyme B release profiles, shown in FIG. 22D, again demonstrates that the doubly modified cells release the most cytokine. Interestingly the levels of Granzyme B expression correlate with the cytotoxicity profiles of the CISH 1 and CISH 2 NK cell groups. Both the CISH 2 NK and CISH 2/CAR19 groups release less Granzyme B than their CISH 1 counterparts, which is reflected in the longer term cytotoxicity data of FIG. 20B, suggesting that reduced CISH expression may be inversely related to Granzyme B release. Finally, FIG. 22E shows release of perforin, which is significantly higher for all NK cell groups, and does not reflect the same patterns seen in FIGS. 22A-22D, suggesting perforin is not a cytotoxicity-limiting cytokine, in these embodiments. However, these data do confirm that immune cells that are subjected to the combination of gene editing (e.g., to reduce expression of an inhibitory factor expressed by the immune cell or to reduce the ability of the immune cell to respond to an inhibitory factor) and the engineering of the cell to express a chimeric cytotoxic signaling complex (such as, for example, a cytotoxicity inducing CAR). In several embodiments, the doubly modified cells exhibit a more robust (e.g., cytotoxicity-inducing) cytokine profile and/or show increased viability/persistence, which allows a greater overall anti-tumor effect, as in accordance with several embodiments disclosed herein. In several embodiments, the double modification of immune cells therefore leads to an overall more efficacious cancer immunotherapy regime, whether using NK cells, T cells, or combinations thereof. Additionally, as discussed above, in several embodiments, the doubly modified cells are also modified in order to reduce their alloreactivity, thereby allowing for a more efficacious allogeneic cell therapy regimen.

CBLB is an E3 ubiquitin ligase that is known to limit T cell activation. In order to determine if disruption of expression of CBLB by NK cells could elicit a more robust anti-tumor response from engineered NK cells, as discussed above, CRISPR/Cas9 was used to disrupt expression of CBLB, though in additional embodiments, other gene editing approaches can be used.

Non-limiting examples of CBLB-targeting guide RNAs are shown below in Table 4.

TABLE 4
CBLB Guide RNAs

| SEQ ID NO: | Name | Sequence | Target |
|---|---|---|---|
| 164 | CBLB-1 | TAATCTGGTGGACCTCATGAAGG | Exon 5 |
| 165 | CBLB-2 | TCGGTTGGCAAACGTCCGAAAGG | Exon 10 |
| 166 | CBLB-3 | AGCAAGCTGCCGCAGATCGCAGG | Exon 2 |

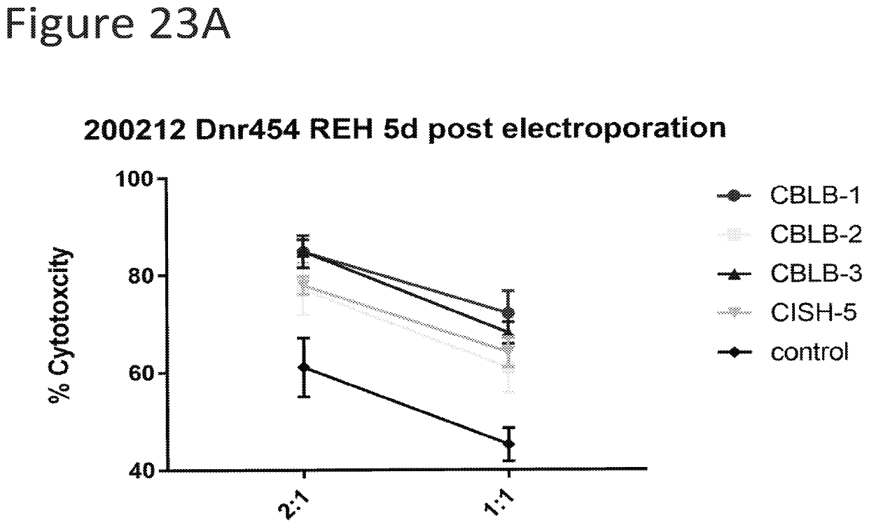

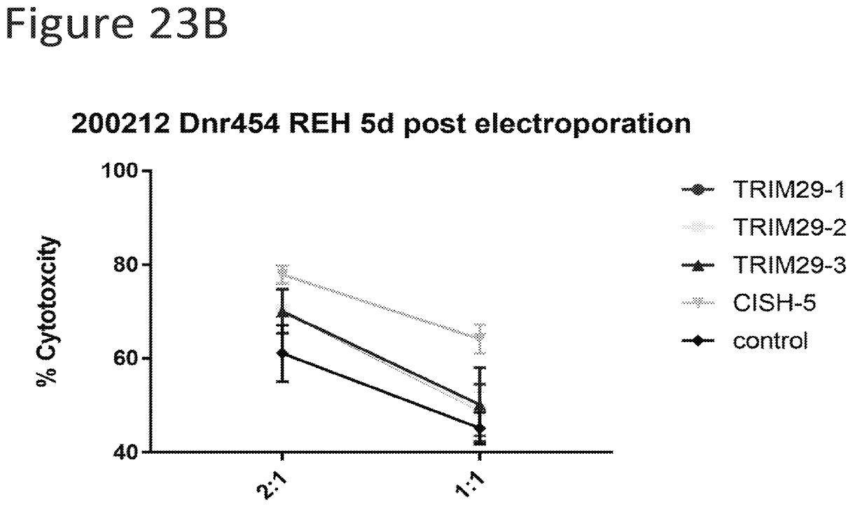

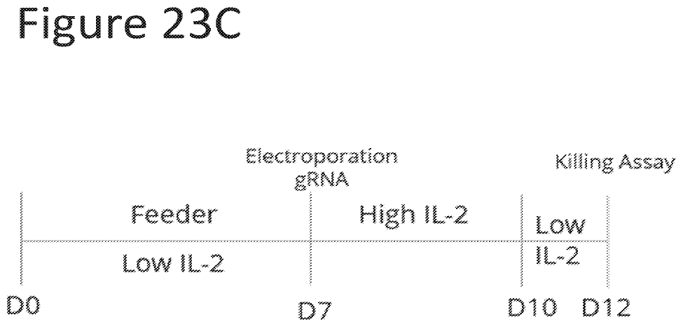

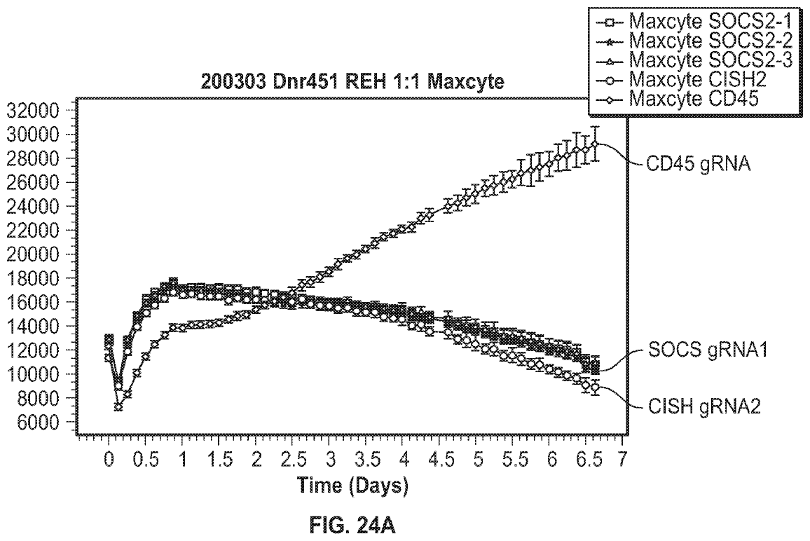

As with the NKG2A and CISH knockout NK cells, Cbl proto-oncogene B (CBLB) knockout (using the guide RNAs shown in Table 4 [SEQ ID NO: 164, 165, 166]) and CISH knockout (using CISH guide RNA 5 [SEQ ID NO: 157]) gene edited NK cells were challenged with Reh tumor cells at a 1:1 and 2:1 E:T ratio 5 days after being electroporated with the gene editing machinery. Briefly, parent NK cells were maintained in a low IL-2 media with feeder cells for 7 days, electroporated on day 7, incubated in high IL-2 media on days 7-10, low IL-2 media on days 10-12, then subjected to the Reh tumor challenge assay on day 12 (FIG. 23C). FIG. 23A shows that while mock NK cells exhibited ˜45% cytotoxicity against Reh cells at the 1:1 ratio, each of the CBLB gRNA knockout NK cell groups showed ˜20% greater cytotoxicity, with an average of ˜70% cytotoxicity against Reh cells. For the 2:1 ratio, the corresponding enhanced cytotoxicity is similar to the 1:1 ratio group, with mock NK cells exhibiting ˜60% cytotoxicity, and each of the CBLB knockout NK cell groups showing a ˜20% greater cytotoxicity, with an average of 80% cytotoxicity against Reh cells. The CISH gRNA 5 knockout NK cell group also exhibited similar results, with approximately 65% in the 1:1 ratio and approximately 80% in the 2:1 ratio, consistent with the previous CISH knockout experiment using gRNAs 1 and 2, discussed above. Overall, the increase in cytotoxicity in CBLB knockout NK cells is proportionate with the CISH knockout NK cells. These data shows that CBLB knockout, in accordance with several embodiments disclosed herein, has a positive impact on NK cell cytotoxicity. In several embodiments, combinations of CISH knockout and CBLB knockout are used to further enhance the cytotoxicity of engineered NK cells. In several embodiments, CBLB knockout NK cells exhibit a greater responsiveness to cytokine stimulation, leading, in part to their enhanced cytotoxicity. In several embodiments, the CBLB knockout leads to increased resulting in increased secretion of effector cytokines like IFN-g and TNF-a and upregulation of the activation marker CD69. In several embodiments, knockout of CBLB is employed in conjunction with engineering the NK cells to express a CAR, leading to further enhancement of NK cell cytotoxicity and/or persistence.

Another E3 ubiquitin ligase, TRIpartite Motif-containing protein 29 (TRIM29), is a negative regulator of NK cell functions. TRIM29 is generally not expressed by resting NK cells, but is readily upregulated following activation (in particular by IL-12/IL-18 stimulation). As discussed above, CRISPR/Cas9 was also used to disrupt expression of TRIM29, though in additional embodiments, other gene editing approaches can be used. Non-limiting examples of TRIM29-targeting guide RNAs are shown below in Table 5.

TABLE 5
TRIM29 Guide RNAs

| SEQ | | | |
|---|---|---|---|
| ID NO: | Name | Sequence | Target |
| 167 | TRIM29-1 | GAACGGTAGGTCCCCTCTCGTGG | Exon 4 |
| 168 | TRIM29-2 | AGCTGCCTTGGACGACGGGCAGG | Exon 7 |
| 169 | TRIM29-3 | TGAGCCGTAACTTCATTGAGAGG | Exon 4 |

TRIM29 knockout (using the gRNAs shown in Table 5 [SEQ ID NO: 167, 167, 169]) gene edited NK cells were challenged with Reh tumor cells at a 1:1 and 2:1 E:T ratio 5 days after being electroporated with the gene editing machinery. The timeline and culture parameters were the same as the CBLB knockout example (FIG. 23C). FIG. 23B shows that TRIM29 knockout has a somewhat less robust impact on enhancing cytotoxicity compared to the CISH or CBLB knockouts. Each of the TRIM29 gRNA NK cell groups had cytotoxicity against Reh cells slightly better than mock cells (˜50% vs ˜45% cytotoxicity at the 1:1 ratio and ˜70% vs ˜60% cytotoxicity at the 2:1 ratio). Comparatively, NK cells transfected with the CISH gRNA 5 had improved cytotoxicity relative to both mock and TRIM29 knockout NK cells in both 1:1 and 2:1 ratio. While, these results indicate that TRIM29 only had a minor effect or no effect on NK cell cytotoxicity under these conditions, that may be at least in part due to the target cell type (e.g., the pathways altered in response to changes in TRIM29 expression are not as active as, for example those altered by changes in CBLB expression). In addition, in several embodiments, a combination of engineering the NK cells with a CAR construct, for example a CAR targeting CD19 and knocking out TRIM29 expression results in significantly enhanced NK cell cytotoxicity and/or persistence. In several embodiments, knockout of TRIM29 expression upregulates interferon release by NK cells.

Interleukins, in particular interleukin-15, are important in NK cell function and survival. Suppressor of cytokine signaling (SOCS) proteins are negative regulators of cytokine release by NK cells. The protein tyrosine phosphatase CD45 is an important regulator of NK cell activity through Src-family kinase activity. CD45 expression is involved in ITAM-specific NK-cell functions and processes such as degranulation, cytokine production, and expansion. Thus, knockout of CD45 expression should result in less effective NK cells. As discussed above, CRISPR/Cas9 was used to disrupt expression of CD45 and SOCS2, though in additional embodiments, other gene editing approaches can be used. Non-limiting examples of CD45 and SOCS2-targeting guide RNAs are shown below in Table 6.

TABLE 6
CD45 and SOCS2 Guide RNAs

| SEQ ID NO: | Name | Sequence | Target |
|---|---|---|---|
| 170 | CD45-1 | AGTGCTGGTGTTGGGCGCAC | Exon 25 |
| 171 | SOCS2-1 | GTGAACAGTGCCGTTCCGGGGGG | Exon 3 |
| 172 | SOCS2-2 | GGCACCGGTACATTTGTTAATGG | Exon 3 |
| 173 | SOCS2-3 | TTCGCCAGACGCGCCGCCTGCGG | Exon 2 |

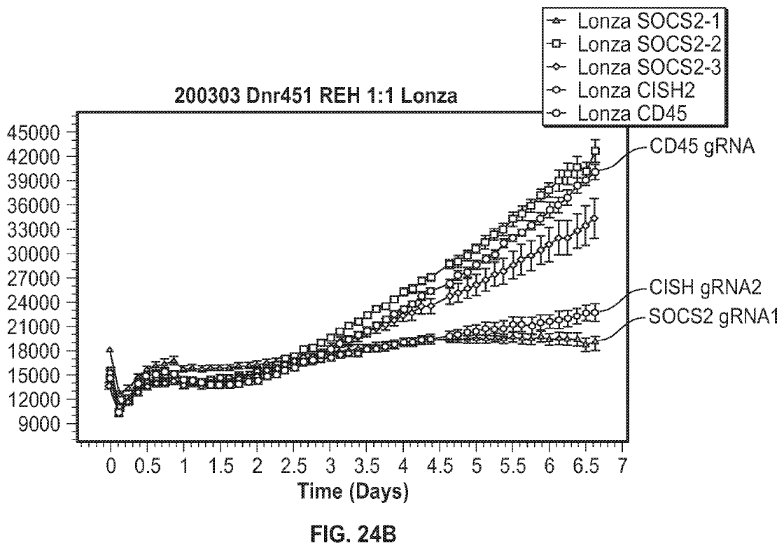

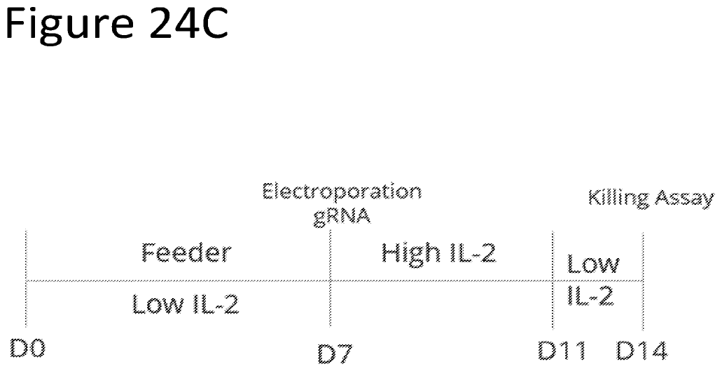

Suppressor of cytokine signaling 2 (SOCS2) knockout (using the gRNAs showed in Table 6 [SEQ ID NO: 171, 172, 173]) gene edited NK cells were assessed in a time course cytotoxicity assay 7 days after being electroporated with the gene editing machinery. Briefly, parent NK cells were maintained in a low IL-2 media with feeder cells for 7 days, electroporated on day 7, incubated in high IL-2 media for days 7-11, low IL-2 media on days 11-14, then subjected to the Incucyte cytotoxicity assay against Reh cells at a 1:1 E:T ratio on day 14 (FIG. 24C). FIG. 23A shows the results of the cytotoxicity assay with NK cells electroporated with a first electroporation system. Using this system, NK cells transfected with each of the SOCS2 gRNAs exhibited cytotoxic activity similar to the CISH gRNA 2 NK cell group (described above). The three gRNA curves for SOCS2 are superimposed in FIG. 24A. CD45 knockout NK cells served as the negative control (as discussed above, CD45 is a positive regulator of NK cell activity, so the CD45 knockout should show reduced cytotoxicity). FIG. 23B shows the results of the cytotoxicity assay with NK cells following the same schedule but electroporated with a second electroporation system. In this case, out of the SOCS2 gRNAs examined, SOCS2 gRNA 1 resulted in an improved cytotoxicity against Reh cells. SOCS2 gRNA 2 and 3 yielded less effective NK cells than with the first electroporation system. SOCS2 gRNA 1 knockout NK cells showed a slight enhancement in cytotoxicity compared to CISH gRNA 2 knockout NK cells. These results indicate that, according to several embodiments, knockout of SOCS2 reduces the negative regulation of NK cells and yield NK cells with enhanced cytotoxicity. In several embodiments, specific gRNAs are used to enhance the cytotoxic NK cells, for example SOCS2 gRNA 1. In several embodiments, knockout of SOCS2 is employed in conjunction with engineering the NK cells to express a CAR, leading to further enhancement of NK cell cytotoxicity and/or persistence.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% sequence identity or homology includes 96%, 97%, 98%, 99%, and 100% sequence identity or homology to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence. Any titles or subheadings used herein are for organization purposes and should not be used to limit the scope of embodiments disclosed herein.

### Sequences

In several embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, or other types of modifications.

In accordance with some embodiments described herein, any of the sequences may be used, or a truncated or mutated form of any of the sequences disclosed herein (and/or included in the accompanying sequence listing) may be used and in any combination.

1. A population of genetically engineered natural killer (NK) cell for cancer immunotherapy, comprising:
a plurality of NK cells,
wherein the plurality of NK cells are engineered to express a cytotoxic receptor comprising an extracellular ligand binding domain, a transmembrane domain, and a cytotoxic signaling complex,
wherein the cytotoxic signaling complex comprises an OX-40 subdomain and a CD3zeta subdomain,
wherein the NK cells are engineered to express membrane bound IL-15,
wherein the NK cells are genetically edited to express reduced levels of a cytokine-inducible SH2-containing (CIS) protein encoded by a CISH gene as compared to a non-engineered NK cell,
wherein the reduced CIS expression was engineered through editing of a CISH gene,
and wherein the genetically engineered NK cells exhibit one or more of enhanced expansion capability, enhanced cytotoxicity against target cells, and enhanced persistence, as compared to NK cells expressing native levels of CIS.

2.-67. (canceled)
